(19) United States
(12) Patent Application Publication
(10) Pub. No.: US 2009/0062401 A1
(43) Pub. Date: Mar. 5, 2009

(54) DECALINE-DERIVED COMPOUNDS AS PHARMACEUTICALLY ACTIVE AGENTS

(75) Inventors: Alex Odermatt, Oberwil (CH); Herbert Waldmann, Dortmund (DE); Michael Scheck, Knittlingen (DE); Marcus A. Koch, Dusseldorf (DE)

Correspondence Address: AMIN, TUROCY & CALVIN, LLP, 127 Public Square, 57th Floor, Key Tower, CLEVELAND, OH, 44114, US

(73) Assignees: UNIVERSITAT DORTMUND, Dortmund (DE); MAX-PLANCK-GESELLSCHAFT ZUR FODERUNG DER WISSENSCHAFTEN E.V., Munich (DE); UNIVERSITAT BERN, Bern (DE)

(21) Appl. No.: 11/813,129

(22) PCT Filed: Dec. 28, 2005
(86) PCT No.: PCT/EP2005/014100

§ 371 Date: Sep. 25, 2008

(30) Foreign Application Priority Data
Dec. 31, 2004 (EP) 04031076.5

(51) Int. Cl. A61K 31/122; C07C 49/733; A61P 31/06; A61P 3/10; A61P 35/00; A61P 25/28; A61P 9/00; A61P 5/44; A61P 3/00; A61P 17/02; A61P 31/00; A61P 3/04; C07C 49/747; C07C 49/743

(52) U.S. Cl. 514/681; 568/374; 568/327; 514/691

(57) ABSTRACT

The present invention relates to compounds having a decaline scaffold, pharmaceutically acceptable salts of these compounds and pharmaceutical compositions containing at least one of these compounds together with pharmaceutically acceptable carrier, excipient and/or diluents. Said decaline-derived compounds can be used for prophylaxsis and/or treatment of diabetes mellitus type I, diabetes mellitus type II, tuberculosis and other infectious diseases, proliferative diseases, cancer, neurodegenerative diseases, obesity, cognitive dysfunctions and metabolic syndromes.

The present invention relates to compounds having a decaline scaffold, and stereoisomeric forms, prodrugs, solvates, hydrates and/or pharmaceutically acceptable salts of these compounds as well as pharmaceutical compositions containing at least one of these decaline derivatives together with pharmaceutically acceptable carrier, excipient and/or diluents. Said decaline-derived compounds are useful for prophylaxsis and/or treatment of diabetes mellitus type I, diabetes mellitus type II, tuberculosis and other infectious diseases, proliferative diseases, cancer, neurodegenerative diseases, obesity, cognitive dysfunctions and metabolic syndromes.

### BACKGROUND OF THE INVENTION

U.S. Pat. No. 6,482,851 B1 discloses dysidiolide compounds and a process for the preparation of said compounds. Furthermore, it is stated that these compounds show an inhibitory effect on the positive cell cycle regulator Cdc25 and the kinases Cdk4, Cdk2 and Cdc2. Said dysidiolide compounds exhibit the potential to act as anti-cancer drugs, since the growth of a number of human tumor cell lines was inhibited with IC₅₀ values ranging from 0.5 to 4.5 μM.

It is object of the present invention to provide compounds and/or pharmaceutically acceptable salts thereof which can be used as pharmaceutically active agents, especially for prophylaxis and/or treatment of diabetes mellitus type I, diabetes mellitus type II, tuberculosis and other infectious diseases, proliferative diseases, cancer, neurodegenerative diseases, obesity, cognitive dysfunctions and metabolic syndromes, as well as compositions comprising at least one of those compounds and/or pharmaceutically acceptable salts thereof as pharmaceutically active ingredients.

The object of the present invention is solved by the teaching of the independent claims. Further advantageous features, aspects and details of the invention are evident from the dependent claims, the description, and the examples of the present application.

The novel decaline derivatives according to the present invention are represented by the following general formula (I)

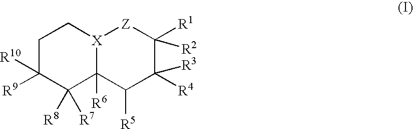

wherein
the moiety

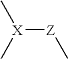

represents

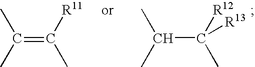

R¹ represents hydrogen and R² is —OH, —OR²¹, —NR¹⁴R¹⁵, or
R¹ and R² form together one of the residues ═O, ═NR¹⁶, or ═CR¹⁷R¹⁸;
R³ and R⁴ are hydrogen or form together the residue ═CR¹⁹R²⁰;
R⁵ and R⁶ represent independently of each other linear or branched, substituted or unsubstituted C₁-C₆-alkyl, linear or branched, substituted or unsubstituted C₂-C₆-alkenyl, —H, -Ph, —CH₂-Ph;
R⁷ represents hydrogen and R⁸ is one of —OH, —OR²¹, —NR²²R²³, or
R⁷ and R⁸ form together the residues ═O;
R⁹ and R¹⁰ are hydrogen or form together the residue ═CR²⁴R²⁵;
R¹¹, R¹², R¹³ represent independently of each other —H, linear or branched, substituted or unsubstituted C₁-C₆-alkyl, —CF₃, —CH₂—CO—O-(linear or branched, substituted or unsubstituted C₁-C₆-alkyl) or

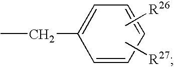

R¹⁴ and R¹⁵ represent independently of each other —H, linear or branched, substituted or unsubstituted C₁-C₆-alkyl, substituted or unsubstituted C₁-C₁₀-cycloalkyl, or

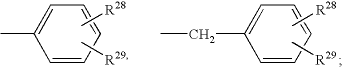

R¹⁶ represents —H or linear or branched, substituted or unsubstituted C₁-C₆-alkyl;
R¹⁷ and R¹⁸ represent independently of each other —H, linear or branched, substituted or unsubstituted C₁-C₆-alkyl, —CO—O-(linear or branched, substituted or unsubstituted C₁-C₆-alkyl), -Ph;
R¹⁹ and R²⁰ represent independently of each other —H, linear or branched, substituted or unsubstituted C₁-C₂₀-alkyl, substituted or unsubstituted heterocyclyl, substituted or unsubstituted aryl, substituted or unsubstituted heteroaryl, substituted or unsubstituted C₁-C₁₀-cycloalkyl

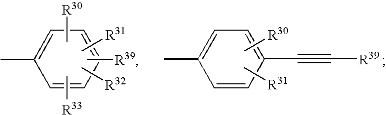

R²¹ represents —CF₃, linear or branched, substituted or unsubstituted C₁-C₂₀-alkyl;
R²² and R²³ represent independently of each other —H, linear or branched, substituted or unsubstituted C₁-C₂₀-alkyl, substituted or unsubstituted C₁-C₁₀-cycloalkyl, —CH₂-Ph;
R²⁴ and R²⁵ represent independently of each other —H, substituted or unsubstituted aryl, substituted or unsubstituted heteroaryl, linear or branched, substituted or unsubstituted C₁-C₂₀-alkyl;
R²⁶— R³⁸ represent independently of each other linear or branched, substituted or unsubstituted C₁-C₂₀-alkyl, —H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N (cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NH-cyclo-C₃H₅, —NH—CS—NHC₃H₇, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —NH—C(═NH)—NH₂, —NH—C(═NH)—NHCH₃, —NH—C(═NH)—NHC₂H₅, —NH—CS—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —NH—CS—NHCH₃, —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F—CHF₂, —CF₃, —CH₂Cl, —CHCl₂, —CCl₃, —CH₂Br —CHBr₂, —CBr₃, —CH₂₁—CH₁₂, —Cl₃, —CH₂—CH₂F—CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₂—CH₂₁, —CH₂—CH₁₂, —CH₂—Cl₃, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C═CH, —Si(CH₃)₃, linear or branched, substituted or unsubstituted heterocyclyl, linear or branched, substituted or unsubstituted C₁-C₁₀-cycloalkyl or two vicinal residues of R²⁶— R³⁸ form together a linear or branched, substituted or unsubstituted heterocyclyl or linear or branched, substituted or unsubstituted C₁-C₁₀-cycloalkyl ring;

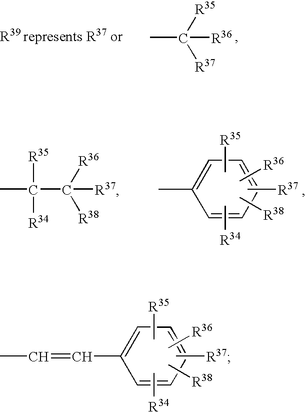

and stereoisomeric forms, prodrugs, solvates, hydrates and/or pharmaceutically acceptable salts thereof.

As used herein, the term “linear or branched C₁-C₆-alkyl” refers to —CH₃, —C₂H₅, —C₃H₇, —CH(CH₃)₂, —C₄H₉, —CH₂—CH(CH₃)₂, —CH(CH₃)—C₂H₅, —C(CH₃)₃, —CH(CH₃)—C₃H₇, —CH₂—CH(CH₃)—C₂H₅, —CH(CH₃)—CH(CH₃)₂, —C₅H₁₁, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃.

Preferred are —CH₃, —C₂H₅, —C₃H₇, —CH(CH₃)₂, —C₄H₉, —CH₂—CH(CH₃)₂, —CH(CH₃)—C₂H₅, —C(CH₃)₃, and —C₅H₁₁. Especially preferred are —CH₃, —C₂H₅, —C₃H₇.

The term “linear or branched C₁-C₂₀-alkyl” preferably refers to “linear or branched C₁-C₆-alkyl” and —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, —C₂₀H₄₁.

As used herein, the term “C₁-C₁₀-cycloalkyl” refers to

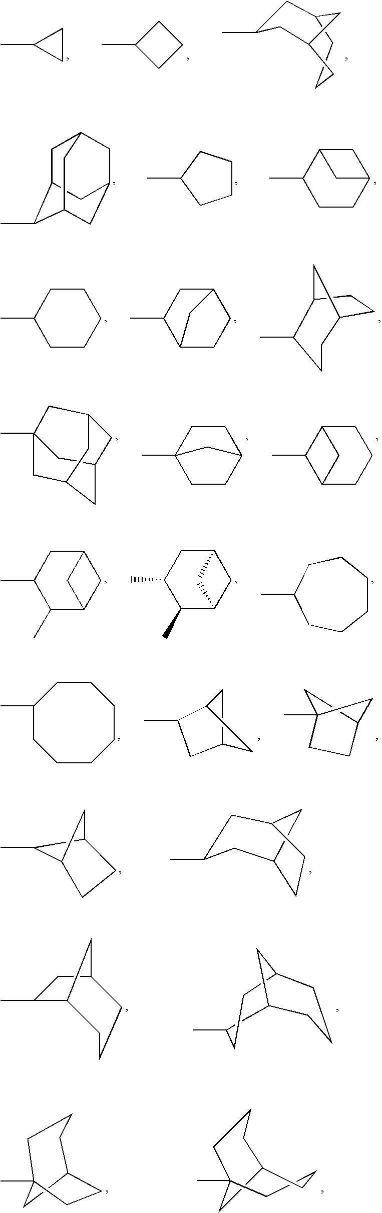

Preferred are the following cycloalkyls:

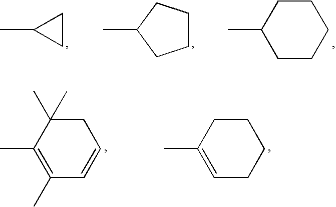

As used herein, the term “linear or branched C₂-C₆-alkenyl” refers to —CH═CH₂, —CH₂—CH═CH₂, —CH═CH—CH₃, —C₂H₄—CH═CH₂, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, —CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH₂—CH═CH—CH₃, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —C(CH₃)═CH₂, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₂H₅)═CH—CH₃, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH═CH—C(CH₃)₃, —CH₂—CH═CH—CH═CH—CH₃, —CH═CH—CH₂—CH═CH—CH₃, —C[CH(CH₃)(C₂H₅)]═CH₂, —CH═CH—CH═CH—C₂H₅, —CH₂—CH═CH—C(CH₃)═CH₂, —C(C₂H₅)═CH—C₂H₅, —CH₂—CH═C(CH₃)—CH═CH₂, —CH₂—C(CH₃)═CH—CH═CH₂, —C(C₂H₅)═C(CH₃)₂, —CH(CH₃)—CH═CH—CH═CH₂, —CH═CH—CH₂—C(CH₃)═CH₂, —C(C₄H₉)═CH₂, —CH═CH—CH(CH₃)—CH═CH₂, —CH═C(CH₃)—CH₂—CH═CH₂, —C[C(CH₃)₃]═CH₂, —CH═C(CH₃)—CH═CH—CH₃, —C(CH₃)═CH—CH₂—CH═CH₂, —CH(C₂H₅)—CH═CH—CH₃, —CH═CH—C(CH₃)═CH—CH₃, —CH═CH—CH═C(CH₃)₂, —C(C₃H₇)═CH—CH₃, —C(CH₃)═CH—CH═CH—CH₃, —CH═C(CH₃)—C(CH₃)═CH₂, —CH₂—C(C₃H₇)═CH₂, —C(CH₃)═CH—C(CH₃)═CH₂, —C(CH₃)═C(CH₃)—CH═CH₂, and —CH═CH—CH═CH—CH═CH₂.

Preferred are —CH═CH₂, —CH₂—CH═CH₂, —C(CH₃)═CH₂, —CH═CH—CH₃, —C₂H₄—CH═CH₂, —CH₂—CH═CH—CH₃. Especially preferred are —CH═CH₂—CH₂—CH═CH₂.

As used herein, the term “linear or branched C₂-C₆-alkynyl” refers to —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —CH₂—CH(CH₃)—C≡CH, —CH(CH₃)—CH₂—C≡CH, —CH(CH₃)—C≡C—CH₃, —C₄H₈—C≡CH, —C₃H₆—C≡C—CH₃, —C₂H₄—C≡C—C₂H₅, —CH₂—C≡C—C₃H₇, —C₂H₄—CH(CH₃)—C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —C≡C—CH₂—CH(CH₃)₂, —C≡C—C₄H₉, —C≡C—C(CH₃)₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —C(CH₃)(C₂H₅)—C≡CH, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —CH₂—C≡C—CE-C—CH₃, —C≡C—CH₂—C≡C—CH₃, —C≡C—C≡C—C₂H₅, —C≡C—CH(CH₃)—C≡CH, —CH(CH₃)—C≡C—C≡CH, —CH(C≡CH)—CH₂—C≡CH, —C(C≡CH)₂—CH₃, —CH₂—CH(C≡CH)₂, —CH(C≡CH)—C≡C—CH₃. Preferred are —C≡CH and —C≡C—CH₃.

As used herein, the term “aryl” refers to phenyl, indenyl, indanyl, naphthyl, 1,2-dihydro-naphthyl, 2,3-dihydronaphthyl, 1,2,3,4-tetrahydronaphthyl (tetralinyl), fluorenyl, anthryl (anthracenyl), 9,10-dihydroanthryl, 1,2,3,4-tetrahydro-anthryl, 1,2,3,4,5,6,7,8-octahydro-anthryl, azulenyl, diphenylmethyl, benzyl, triphenylmethyl (trityl), styryl, naphthoquinonyl, acenaphthyl, anthraquinonyl, phenanthryl (phenanthrenyl) and especially to a mono- or bicyclic 6 to 10 membered ring system, preferably phenyl or napthyl.

As used herein, the term “heteroaryl” refers to heteroaromatic groups which have from 5 to 10 ring atoms, from 1 to 4 of which are selected from O, N and/or S. Preferred groups have 1 or 2 heteroatoms in a 5- or 6-membered aromatic ring. Mono and bicyclic ring systems are included. Typical heteroaryl groups which are at least partially aromatic include pyridyl, furyl, thienyl, pyrrolyl, oxazolyl, thiazolyl, imidazolyl, isoxazolyl, isothiazolyl, oxadiazolyl, pyridazinyl, pyrimidyl, pyrazinyl, 1,3,5-triazinyl, 1,2,3-triazolyl, 1,3,4-thiadiazolyl, indolizinyl, indolyl, isoindolyl, benzo[b]furyl, thiophenyl, benzo[b]thienyl, indazolyl, benzimidazolyl, benzthiazolyl, thiazolyl, purinyl, quinolizinyl, quinolyl, isoquinolyl, quinazolinyl, quinoxalinyl, 1,8-naphthyridinyl, tetrahydroquinolyl, benzooxazolyl, chrom-2-onyl, indazolyl, indenyl. Said heteroaryl groups may further be substituted by one, two, three, four, five or more substituents selected from the group consisting of R²⁶-R³⁸, linear or branched C₁-C₂₀-alkyl, C₁-C₁₀-cycloalkyl, linear or branched C₂-C₆-alkenyl, linear or branched C₂-C₆-alkynyl and aryl.

Preferred heterocyclic group are:

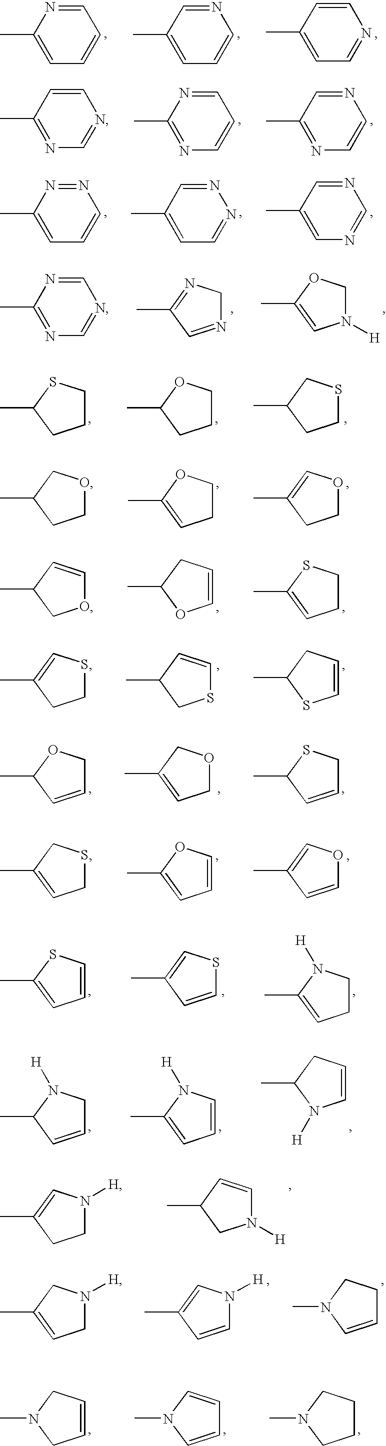

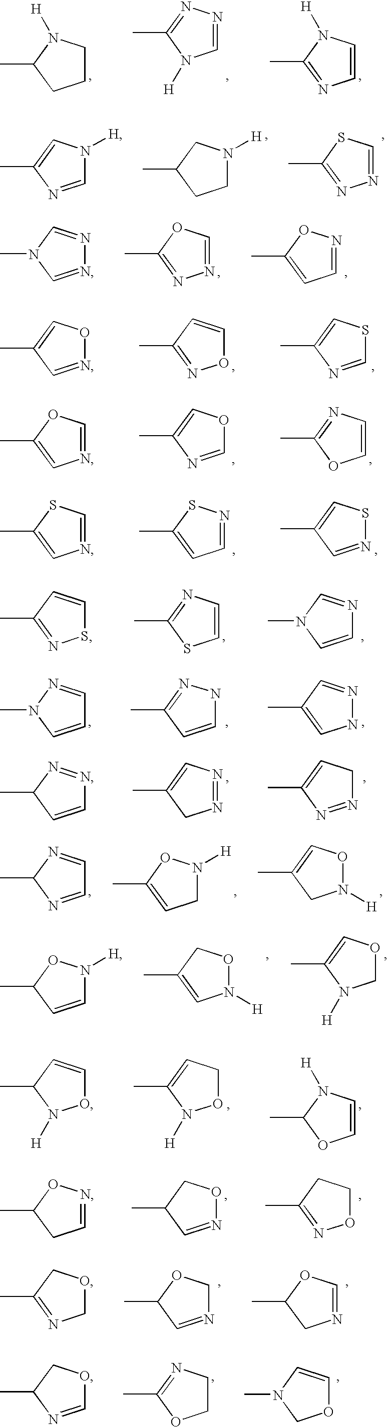

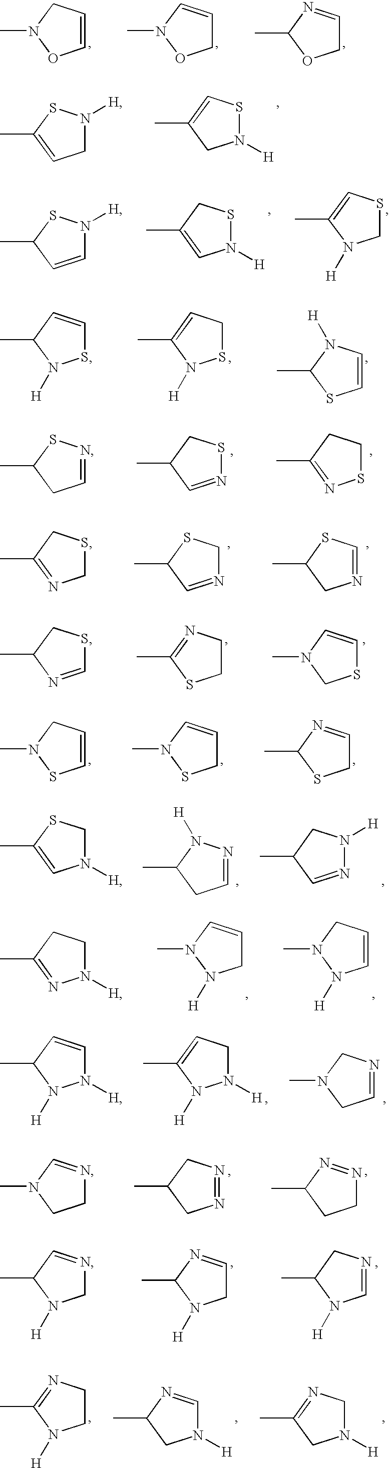

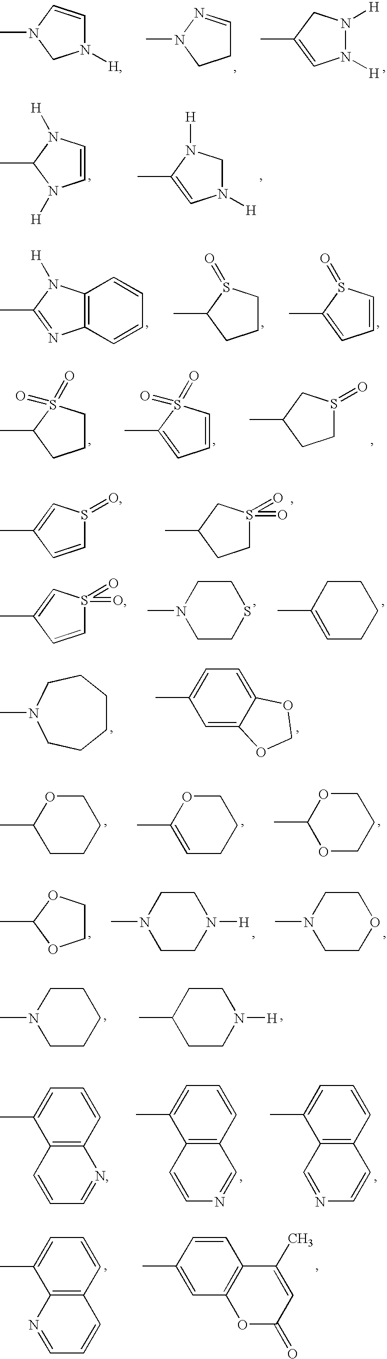

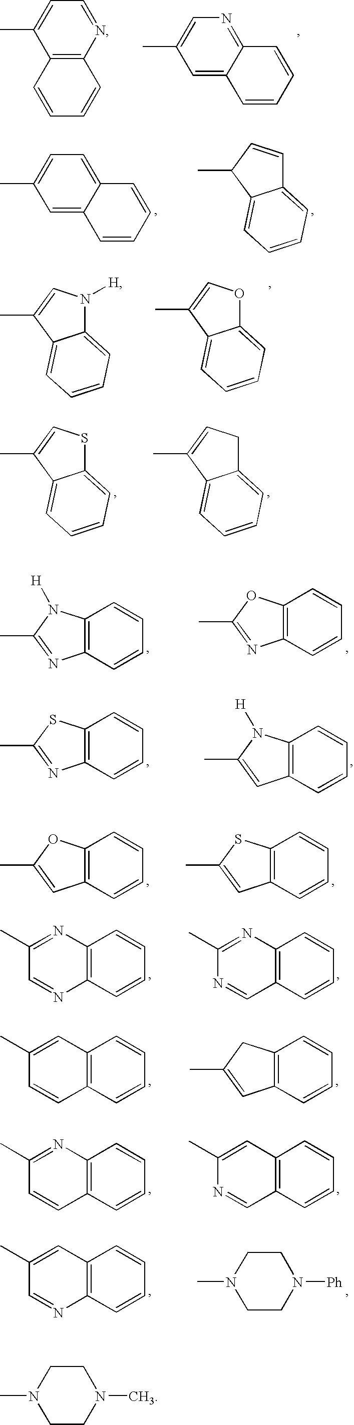

Said preferred heteroaryl groups may further be substituted by one, two, three, four, five or more substituents selected from the group consisting of R²⁶— R³⁸ linear or branched C₁-C₂₀-alkyl, C₁-C₁₀-cycloalkyl, linear or branched C₂-C₆-alkenyl, linear or branched C₂-C₆-alkynyl and aryl.

As used herein, the term “heterocyclyl” refers to carbocycles having at least one heteroatom in the ring such as oxygen, nitrogen, or sulfur. Such heterocycles may be saturated or partially unsaturated but not aromatic. Examples for heterocyclic residues are 1,3-dioxolane, benzo[1,3]dioxolyl, pyrazolinyl, pyranyl, thiomorpholinyl, pyrazolidinyl, piperidyl, piperazinyl, 1,4-dioxanyl, imidazolinyl, pyrrolinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranl, pyrrolidinyl, tetrahydrothiophenyl, tetrahydrofuranyl.

The term “substituted” or “substituted alkyl”, “substituted cycloalkyl”, “substituted heterocyclyl”, “substituted aryl”, “substituted heteroaryl”, respectively shall refer to the addressed residue such as “alkyl”, “cycloalkyl”, “heterocyclyl”, “aryl”, or “heteroaryl” bearing one, two, three, four, five or more, preferably one or two substituents independently selected from the following group: —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F—CHF₂, —CF₃, —CH₂Cl, —CHCl₂, —CCl₃, —CH₂Br —CHBr₂, —CBr₃, —CH₂₁—CH₁₂, —Cl₃, —CH₂—CH₂F—CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₂—CH₂₁, —CH₂—CH₁₂, —CH₂—Cl₃, -cyclo-C₃H₅, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C═CH, —CH₃, —C₂H₅, —C₃H₇, —CH(CH₃)₂, —C₄H₉, —CH₂—CH(CH₃)₂, —CH(CH₃)—C₂H₅, —C(CH₃)₃, —CH(CH₃)—C₃H₇, —CH₂—CH(CH₃)—C₂H₅, —CH(CH₃)—CH(CH₃)₂, —C₅H₁₁, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃.

The term “—CO—O-(linear or branched, substituted or unsubstituted C₁-C₆-alkyl)” refers to the residue “linear or branched, substituted or unsubstituted C₁-C₆-alkyl” connected to the group “—CO—O—” through the oxygen atom. Accordingly, the term “—CH₂—CO—O-(linear or branched, substituted or unsubstituted C₁-C₆-alkyl)” refers to the residue “linear or branched, substituted or unsubstituted C₁-C₆-alkyl” connected to the group “—CH₂—CO—O—” through the oxygen atom.

The following subformula (IIA)-(IIW) of formula (I) are especially preferred:

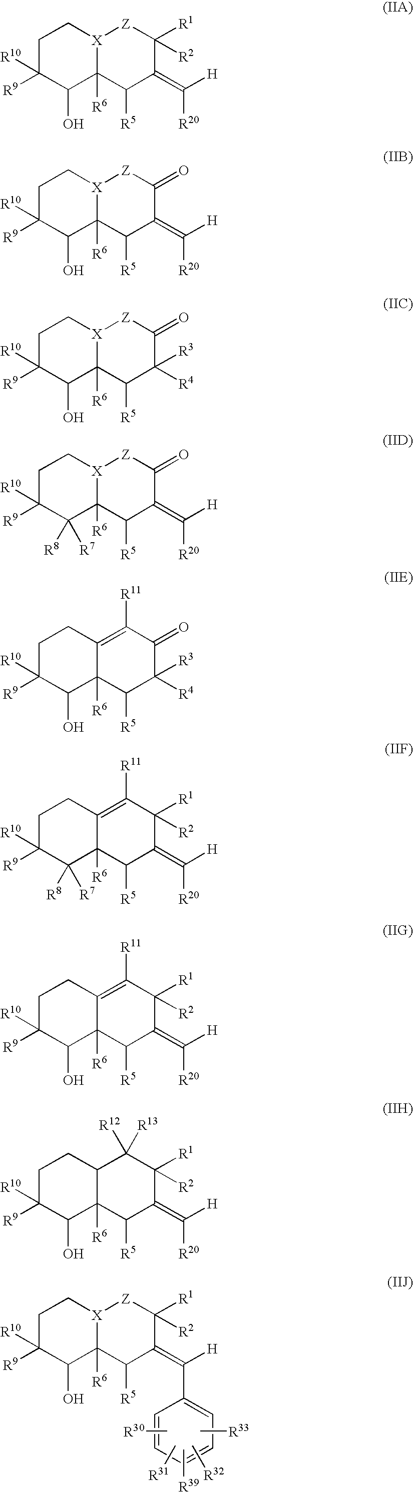

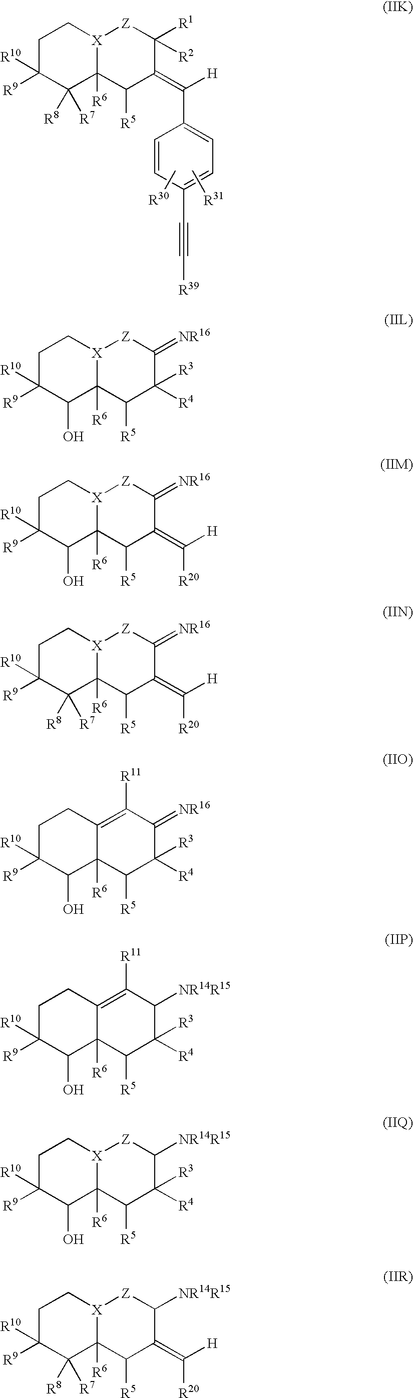

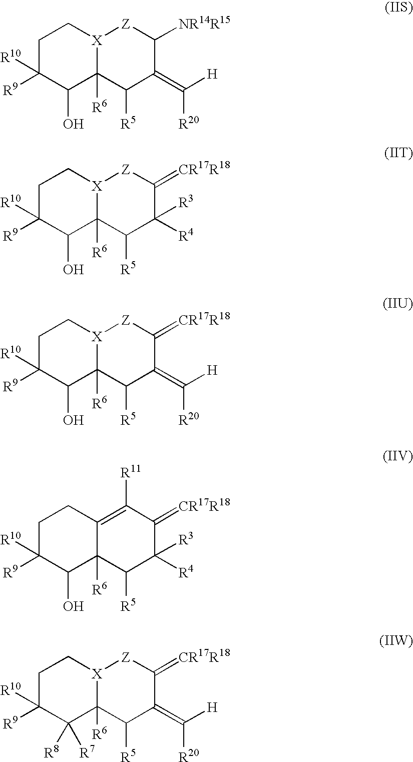

wherein
R¹-R³⁹, X and Z have the meanings as defined above.

Furthermore, the following subformula (IIIA)-(IIIG) of formula (I) are also especially preferred:

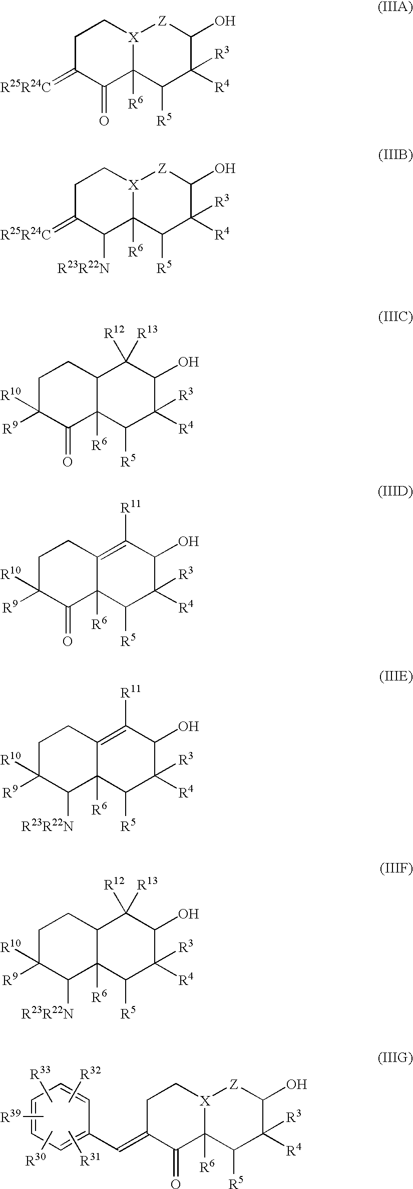

wherein
R¹-R³⁹, X and Z have the meanings as defined above.

Also especially preferred are the compounds of the following general formula (IA)-(IF):

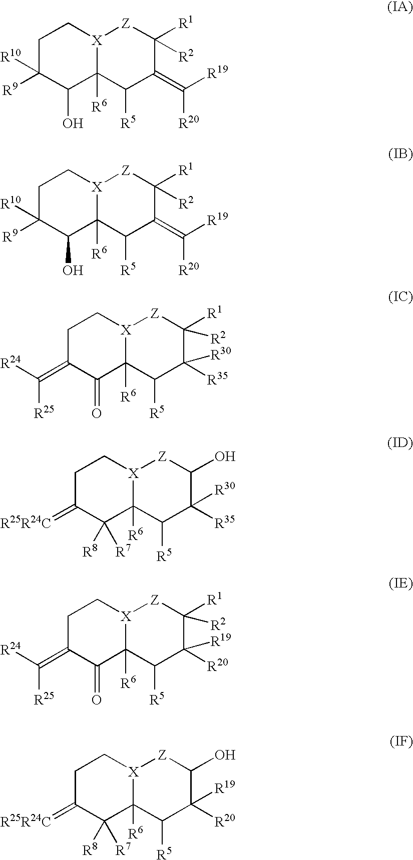

wherein
R¹-R³⁹, X and Z have the meanings as defined above.

In yet another preferred embodiment of the present invention the compound according to the general formula (I) is selected from the group of compounds depicted in Table 1.

TABLE I
Claimed compounds according to the present invention

| | | | LC ret. | |
|---|---|---|---|---|
| | | | time | MS |
| No. | MW | IUPAC name | [min] | [M + H]+ |
| 1 | 318.14 | (3E,4aS,5S)-3-(3,4-Difluorbenzyliden)-4,4a,5,6,7,8- | 2.33 | 318.15 |
| | | hexahydro-5-hydroxy-1,4a-dimethyl-naphthalen-2(3H)-one | | |
| 2 | 298.16 | (3E,4aS,5S)-3-(4-Hydroxybenzyliden)-4,4a,5,6,7,8- | 3.05 | 298.17 |
| | | hexahydro-5-hydroxy-1,4a-dimethyl-naphthalen-2(3H)-one | | |
| 3 | 334.11 | (3E,4aS,5S)-3-(2-Chlor-6-fluorbenzyliden)-4,4a,5,6,7,8- | 2.40 | 334.13 |
| | | hexahydro-5-hydroxy-1,4a-dimethyl-naphthalen-2(3H)-one | | |
| 4 | 322.16 | (3E,4aS,5S)-3-[(Benzofuran-2-yl)methylen]-4,4a,5,6,7,8- | 3.09 | 322.15 |
| | | hexahydro-5-hydroxy-1,4a-dimethyl-naphthalen-2(3H)-one | | |
| 5 | 342.18 | (3E,4aS,5S)-3-(3,4-Dimethoxybenzyliden)-4,4a,5,6,7,8- | 2.94 | 342.19 |
| | | hexahydro-5-hydroxy-1,4a-dimethyl-naphthalen-2(3H)-one | | |
| 6 | 372.19 | (3E,4aS,5S)-3-(3,4,5-Trimethoxybenzyliden)-4,4a,5,6,7,8- | 3.01 | 372.20 |
| | | hexahydro-5-hydroxy-1,4a-dimethylnaphthalen-2(3H)-one | | |
| 7 | 316.12 | (3E,4aS,5S)-3-(4-Chlorobenzyliden)-4,4a,5,6,7,8-hexahydro- | 2.18 | 316.10 |
| | | 5-hydroxy-1,4a-dimethylnaphthalen-2(3H)-one | | |
| 8 | 272.14 | (3E,4aS,5S)-3-[(Furan-3-yl)methylen]-4,4a,5,6,7,8-hexahydro- | 2.87 | 272.13 |
| | | 5-hydroxy-1,4a-dimethylnaphthalen-2(3H)-one | | |
| 9 | 366.03 | (3E,4aS,5S)-3-[(4-Bromthiophen-2-yl)methylen]-4,4a,5,6,7,8- | 3.14 | 366.03 |
| | | hexahydro-5-hydroxy-1,4a-dimethylnaphthalen-2(3H)-one | | |
| 10 | 283.16 | (3E,4aS,5S)-4,4a,5,6,7,8-Hexahydro-5-hydroxy-1,4a- | 2.78 | 284.16 |
| | | dimethyl-3-[(pyridin-3-yl)methylen]naphthalen-2(3H)-one | | |
| 11 | 328.15 | (3E,4aS,5S)-3-[4-(Methylthio)benzyliden]-4,4a,5,6,7,8- | 2.69 | 328.16 |
| | | hexahydro-5-hydroxy-1,4a-dimethylnaphthalen-2(3H)-one | | |
| 12 | 322.08 | (3E,4aS,5S)-3-[(4-Chlorthiophen-2-yl)methylen]-4,4a,5,6,7,8- | 3.03 | 322.06 |
| | | hexahydro-5-hydroxy-1,4a-dimethylnaphthalen-2(3H)-one | | |
| 13 | 285.17 | (3E,4aS,5S)-4,4a,5,6,7,8-Hexahydro-5-hydroxy-1,4a-dimethyl- | 2.81 | 285.15 |
| | | 3-[(1-methyl-1H-pyrrol-2-yl)methylen]naphthalen-2(3H)-on | | |
| 14 | 328.17 | (3E,4aS,5S)-3-(2-Hydroxy-4-methoxybenzyliden)-4,4a,5,6, | 3.24 | 328.18 |
| | | 7,8-hexahydro-5-hydroxy-1,4a-dimethylnaphthalen-2(3H)-one | | |
| 15 | 340.20 | (3E,4aS,5S)-3-(2-Ethyl-4-methoxybenzyliden)-4,4a,5,6,7,8- | 2.64 | 340.21 |
| | | hexahydro-5-hydroxy-1,4a-dimethylnaphthalen-2(3H)-one | | |
| 16 | 326.19 | (3E,4aS,5S)-3-(4-Methoxy-2-methylbenzyliden)-4,4a,5,6,7,8- | 2.60 | 326.22 |
| | | hexahydro-5-hydroxy-1,4a-dimethylnaphthalen-2(3H)-one | | |
| 17 | 327.15 | (3E,4aS,5S)-3-(4-Nitrobenzyliden)-4,4a,5,6,7,8-hexahydro-5- | 2.75 | 327.13 |
| | | hydroxy-1,4a-dimethylnaphthalen-2(3H)-one | | |
| 18 | 282.16 | (3E,4aS,5S)-3-Benzyliden-4,4a,5,6,7,8-hexahydro-5-hydroxy- | 2.01 | 286.16 |
| | | 1,4a-dimethyl-naphthalen-2(3H)-one | | |
| 19 | 302.13 | (3E,4aS,5S)-4,4a,5,6,7,8-Hexahydro-5-hydroxy-1,4a-dimethyl- | 2.68 | 302.13 |
| | | 3-[(4-methylthiophen-2-yl)methylen]naphthalen-2(3H)-one | | |
| 20 | 408.30 | (3E,4aS,5S)-3-(2-Methyl-4-octyl-benzyliden)-4,4a,5,6,7,8- | 2.31 | 408.32 |
| | | hexahydro-5-hydroxy-1,4a-dimethyl-naphthalen-2(3H)-one | | |
| 21 | 296.18 | (3E,4aS,5S)-3-(3-Methylbenzyliden)-4,4a,5,6,7,8-hexahydro- | 2.17 | 296.17 |
| | | 5-hydroxy-1,4a-dimethylnaphthalen-2(3H)-one | | |
| 22 | 360.30 | (3E,4aS,5S)-3-Dodecyliden-4,4a,5,6,7,8-hexahydro-5- | 2.34 | 360.32 |
| | | hydroxy-1,4a-dimethylnaphthalen-2(3H)-one | | |
| 23 | 324.21 | (3E,4aS,5S)-3-(4-Ethyl-2-methylbenzyliden)-4,4a,5,6,7,8- | 2.40 | 324.19 |
| | | hexahydro-5-hydroxy-1,4a-dimethylnaphthalen-2(3H)-one | | |
| 24 | 312.17 | (3E,4aS,5S)-3-(4-Methoxybenzyliden)-4,4a,5,6,7,8- | 2.74 | 312.18 |
| | | hexahydro-5-hydroxy-1,4a-dimethylnaphthalen-2(3H)-on | | |
| 25 | 272.14 | (2E,8aS)-2-[(Furan-2-yl)methylen]-3,4,6,7,8,8a-hexahydro-6- | 2.84 | 272.13 |
| | | hydroxy-5,8a-dimethylnaphthalen-1(2H)-one | | |
| 26 | 340.17 | 3-{(1E)-[(4S,4aS)-1,2,3,4,4a,5-Hexahydro-4-hydroxy-4a,8- | 3.14 | 340.17 |
| | | dimethyl-7-oxonaphthalen-6(7H)-yliden]methyl}phenylacetat | | |
| 27 | 366.03 | (3E,4aS,5S)-3-[(5-Bromthiophen-2-yl)methylen]-4,4a,5,6,7,8- | 2.89 | 366.03 |
| | | hexahydro-5-hydroxy-1,4a-dimethylnaphthalen-2(3H)-one | | |
| 28 | 318.14 | (3E,4aS,5S)-3-(2,4-Difluorbenzyliden)-4,4a,5,6,7,8- | 2.74 | 318.13 |
| | | hexahydro-5-hydroxy-1,4a-dimethylnaphthalen-2(3H)-one | | |
| 29 | 246.16 | (3E,4aS,5S)-3-[(E)-But-2-enyliden]-4,4a,5,6,7,8-hexahydro-5- | 2.04 | 246.18 |
| | | hydroxy-1,4a-dimethylnaphthalen-2(3H)-one | | |
| 30 | 288.12 | (3E,4aS,5S)-4,4a,5,6,7,8-Hexahydro-5-hydroxy-1,4a- | 2.51 | 288.11 |
| | | dimethyl-3-[(thiophen-2-yl)methylen]naphthalen-2(3H)-one | | |
| 31 | 340.17 | 4-{(1E)-[(4S,4aS)-1,2,3,4,4a,5-Hexahydro-4-hydroxy-4a,8- | 2.99 | 340.17 |
| | | dimethyl-7-oxonaphthalen-6(7H)-yliden]methyl}phenylacetat | | |
| 32 | 248.18 | (3E,4aS,5S)-3-Butyliden-4,4a,5,6,7,8-hexahydro-5-hydroxy- | 2.10 | 248.19 |
| | | 1,4a-dimethylnaphthalen-2(3H)-one | | |
| 33 | 302.13 | (3E,4aS,5S)-4,4a,5,6,7,8-Hexahydro-5-hydroxy-1,4a-dimethyl- | 2.81 | 302.13 |
| | | 3-[(3-methylthiophen-2-yl)methylen]naphthalen-2(3H)-one | | |
| 34 | 272.14 | (3E,4aS,5S)-3-[(Furan-2-yl)methylen]-4,4a,5,6,7,8-hexahydro- | 2.86 | 272.15 |
| | | 5-hydroxy-1,4a-dimethylnaphthalen-2(3H)-one | | |
| 35 | 283.16 | (3E,4aS,5S)-4,4a,5,6,7,8-Hexahydro-5-hydroxy-1,4a- | 2.70 | 283.14 |
| | | dimethyl-3-[(pyridin-4-yl)methylen]naphthalen-2(3H)-one | | |
| 36 | 382.19 | (3E,4aS,5S)-3-[4-(2-Phenylethinyl)benzyliden]-4,4a,5,6,7,8- | 3.07 | 382.20 |
| | | hexahydro-5-hydroxy-1,4a-dimethylnaphthalen-2(3H)-one | | |
| 37 | 336.17 | (3E,4aS,5S)-3-[4-(2-Methoxy-ethinyl)benzyliden]-4,4a,5,6,7, | 2.91 | 336.18 |
| | | 8-hexahydro-5-hydroxy-1,4a-dimethylnaphthalen-2(3H)-one | | |
| 38 | 406.25 | (3E,4aS,5S)-3-[4-(3-tert-Butoxybut-1-inyl)benzyliden]-4,4a,5,6, | 2.96 | 406.28 |
| | | 7,8-hexahydro-5-hydroxy-1,4a-dimethylnaphthalen-2(3H)-one | | |
| 39 | 386.22 | (3E,4aS,5S)-3-[4-(2-Cyclo-hexenylethinyl)benzyliden]-4,4a,5, | 2.71 | 386.23 |
| | | 6,7,8-hexahydro-5-hydroxy-1,4a-dimethylnaphthalen-2(3H)-on | | |
| 40 | 362.22 | (3E,4aS,5S)-3-[4-(Hex-1-inyl)benzyliden]-4,4a,5,6,7,8- | 2.64 | 362.23 |
| | | hexahydro-5-hydroxy-1,4a-dimethyl-naphthalen-2(3H)-one | | |
| 41 | 396.21 | (3E,4aS,5S)-3-[4-(2-p-Tolylethinyl)benzyliden]-4,4a,5,6,7,8- | 2.78 | 396.19 |
| | | hexahydro-5-hydroxy-1,4a-dimethyl-naphthalen-2(3H)-one | | |
| 42 | 400.24 | (3E,4aS,5S)-3-[4-(Nona-1,8-diinyl)benzyliden]-4,4a,5,6,7,8- | 3.07 | 400.24 |
| | | hexahydro-5-hydroxy-1,4a-dimethyl-naphthalen-2(3H)-one | | |
| 43 | 502.38 | (3E,4aS,5S)-3-[4-(Hexadec-1-inyl)benzyliden]-4,4a,5,6,7,8- | 3.28 | 502.36 |
| | | hexahydro-5-hydroxy-1,4a-dimethyl-naphthalen-2(3H)-one | | |
| 44 | 364.20 | (3E,4aS,5S)-3-[4-(3-Hydroxy-3-methylbut-1-inyl)benzyliden]- | 3.11 | 364.22 |
| | | 4,4a,5,6,7,8-hexahydro-5-hydroxy-1,4a-dimethyl-naphthalen- | | |
| | | 2(3H)-one | | |
| 45 | 446.31 | (3E,4aS,5S)-3-[4-(Dodec-1-inyl)benzyliden]-4,4a,5,6,7,8- | 3.04 | 446.31 |
| | | hexahydro-5-hydroxy-1,4a-dimethyl-naphthalen-2(3H)-on | | |
| 46 | 408.23 | (3E,4aS,5S)-3-[4-(3,3-Diethoxyprop-1-inyl)benzyliden]- | 2.94 | 408.24 |
| | | 4,4a,5,6,7,8-hexahydro-5-hydroxy-1,4a-dimethylnaphthalen- | | |
| | | 2(3H)-on | | |
| 47 | 373.20 | 6-{4-{(1E)-[(43,4aS)-1,2,3,4,4a,5-Hexahydro-4-hydroxy-4a,8- | 2.98 | 373.20 |
| | | dimethyl-7-oxonaphthalen-6(7H)-yliden]-methyl}phenyl}hex-5- | | |
| | | in-nitril | | |
| 48 | 404.23 | (3E,4aS,5S)-3-{4-[2-(1-Hydroxycyclohexyl)ethinyl]benzyliden}- | 3.12 | 404.23 |
| | | 4,4a,5,6,7,8-hexahydro-5-hydroxy-1,4a-dimethylnaphthalen- | | |
| | | 2(3H)-one | | |
| 49 | 400.18 | (3E,4aS,5S)-3-{4-[2-(3-Fluorphenyl)ethinyl]benzyliden}- | 2.79 | 400.16 |
| | | 4,4a,5,6,7,8-hexahydro-5-hydroxy-1,4a-dimethylnaphthalen- | | |
| | | 2(3H)-one | | |
| 50 | 439.25 | (3E,4aS,5S)-3-{4-[3-(N-Ethyl-N-benzylamino)prop-1-inyl]- | 2.98 | 439.26 |
| | | benzyliden}-4,4a,5,6,7,8-hexahydro-5-hydroxy-1,4a- | | |
| | | dimethylnaphthalen-2(3H)-one | | |
| 51 | 363.22 | (3E,4aS,5S)-3-{4-[3-(Dimethylamino)prop-1-inyl]benzyliden}- | 2.86 | 363.22 |
| | | 4,4a,5,6,7,8-hexahydro-5-hydroxy-1,4a-dimethylnaphthalen- | | |
| | | 2(3H)-one | | |
| 52 | 396.21 | (3E,4aS,5S)-3-[4-(3-Phenylprop-1-inyl)benzyliden]-4,4a,5,6,7, | 2.60 | 396.20 |
| | | 8-hexahydro-5-hydroxy-1,4a-dimethylnaphthalen-2(3H)-on | | |
| 53 | 378.20 | (3E,4aS,5S)-3-{4-[2-(Trimethylsilyl)ethinyl]benzyliden}- | 2.92 | 378.20 |
| | | 4,4a,5,6,7,8-hexahydro-5-hydroxy-1,4a-dimethylnaphthalen- | | |
| | | 2(3H)-on | | |
| 54 | 590.34 | (35,4aS,5S)-3-[4-(17-Ethinyl-ostradiol)benzyliden]-4,4a,5,6,7, | 4.22 | 590.31 |
| | | 8-hexahydro-5-hydroxy-1,4a-dimethylnaphthalen-2(3H)-one | | |
| 55 | 350.19 | (3E,4aS,5S)-3-[4-(4-Hydroxybut-1-inyl)benzyliden]-4,4a,5,6,7, | 3.06 | 350.18 |
| | | 8-hexahydro-5-hydroxy-1,4a-dimethylnaphthalen-2(3H)-one | | |
| 56 | 346.19 | (3E,4aS,5S)-3-[4-(3-Methylbut-3-en-1-inyl)benzyliden]-4,4a,5, | 2.71 | 346.20 |
| | | 6,7,8-hexahydro-5-hydroxy-1,4a-dimethylnaphthalen-2(3H)-on | | |
| 57 | 348.21 | (3E,4aS,5S)-3-[4-(3-Methylbut-1-inyl)benzyliden]-4,4a,5,6,7,8- | 2.66 | 348.23 |
| | | hexahydro-5-hydroxy-1,4a-dimethylnaphthalen-2(3H)-one | | |
| 58 | 366.20 | (3E,4aS,5S)-4,4a,5,6,7,8-Hexahydro-5-hydroxy-3-{2- | 4.09 | 366.21 |
| | | [(1S,3R,4S,5R)-1,3,4,5-tetrahydroxycyclohexyl]-ethyliden}- | | |
| | | 1,4a-dimethylnaphthalen2(3H)-one | | |
| 59 | 300.17 | (3E,4aS,5S)-3-[3-(Furan-2-yl)propyliden]-4,4a,5,6,7,8- | 2.91 | 300.16 |
| | | hexahydro-5-hydroxy-1,4a-dimethylnaphthalen-2(3H)-one | | |
| 60 | 298.16 | (3E,4aS,5S)-3-[(E)-3-(Furan-2-yl)allyliden]-4,4a,5,6,7,8- | 2.94 | 298.16 |
| | | hexahydro-5-hydroxy-1,4a-dimethylnaphthalen-2(3H)-one | | |
| 61 | 352.24 | (3E,4aS,5S)-3-(2,3,4,5,6-Pentamethylbenzyliden)-4,4a,5,6, | 2.91 | 352.23 |
| | | 7,8-hexahydro-5-hydroxy-1,4a-dimethylnaphthalen-2(3H)-one | | |
| 62 | 272.14 | (3E,4aS,5S)-3[(Furan-3-yl)methylen]-4,4a,5,6,7,8-hexahydro- | 2.87 | 272.15 |
| | | 5-hydroxy-1,4a-dimethylnaphthalen-2(3H)-one | | |
| 63 | 360.07 | (3E,4aS,5S)-3-(2-Brombenzyliden)-4,4a,5,6,7,8-hexahydro-5- | 2.58 | 360.06 |
| | | hydroxy-1,4a-dimethylnaphthalen-2(3H)-one | | |
| 64 | 360.07 | (3E,4aS,5S)-3-(3-Brombenzyliden)-4,4a,5,6,7,8-hexahydro-5- | 2.64 | 360.06 |
| | | hydroxy-1,4a-dimethylnaphthalen-2(3H)-one | | |
| 65 | 360.07 | (3E,4aS,5S)-3-(4-Brombenzyliden)-4,4a,5,6,7,8-hexahydro-5- | 2.62 | 360.09 |
| | | hydroxy-1,4a-dimethylnaphthalen-2(3H)-one | | |
| 66 | 388.20 | (3E,4aS,5S)-3-[4-(Benzyloxy)benzyliden]-4,4a,5,6,7,8- | 2.91 | 388.21 |
| | | hexahydro-5-hydroxy-1,4a-dimethylnaphthalen-2(3H)-one | | |
| 67 | 408.06 | (3E,4aS,5S)-3-(2-Iodbenzyliden)-4,4a,5,6,7,8-hexahydro-5- | 2.90 | 408.05 |
| | | hydroxy-1,4a-dimethylnaphthalen-2(3H)-one | | |
| 68 | 408.06 | (3E,4aS,5S)-3-(3-Iodbenzyliden)-4,4a,5,6,7,8-hexahydro-5- | 2.98 | 408.08 |
| | | hydroxy-1,4a-dimethylnaphthalen-2(3H)-one | | |
| 69 | 408.06 | (3E,4aS,5S)-3-(4-Iodbenzyliden)-4,4a,5,6,7,8-hexahydro-5- | 3.00 | 408.06 |
| | | hydroxy-1,4a-dimethylnaphthalen-2(3H)-one | | |
| 70 | 326.09 | (3E,4aS,5S)-3-(3-Brombutyliden)-4,4a,5,6,7,8-hexahydro-5- | 2.66 | 326.08 |
| | | hydroxy-1,4a-dimethylnaphthalen-2(3H)-one | | |
| 71 | 346.19 | (3E,4aS,5S)-3-[4-(3-Methylbut-3-en-1-inyl)benzyliden]-4,4a,5, | 2.78 | 346.19 |
| | | 6,7,8-hexahydro-5-hydroxy-1,4a-dimethylnaphthalen-2(3H)-on | | |
| 72 | 424.24 | (3E,4aS,5S)-3-[4-(5-Phenylpent-1-inyl)benzyliden]-4,4a,5,6, | 2.95 | 424.24 |
| | | 7,8-hexahydro-5-hydroxy-1,4a-dimethylnaphthalen-2(3H)-one | | |
| 73 | 388.23 | (3E,4aS,5S)-3-[4-(2-Cyclohexyl-ethinyl)benzyliden]-4,4a,5,6, | 2.86 | 388.23 |
| | | 7,8-hexahydro-5-hydroxy-1,4a-dimethylnaphthalen-2(3H)-one | | |
| 74 | 406.25 | (E,4aS,5S)-3-{4-[2-(1-Hydroxycyclohexyl)ethinyl]-benzyliden}- | 3.02 | 406.22 |
| | | octahydro-5-hydroxy-1,4a-dimethylnaphthalen-2(1H)-one | | |
| 75 | 530.41 | (3E,4aS,5S)-3-[4-(Octadec-1-inyl)benzyliden]-4,4a,5,6,7,8- | 3.67 | 530.40 |
| | | hexahydro-5-hydroxy-1,4a-dimethylnaphthalen-2(3H)-one | | |
| 76 | 486.22 | (3E,4aS,5S)-3-{4-[2-(9-Hydroxy-9H-fluoren-9-yl)ethinyl]- | 3.24 | 486.25 |
| | | benzyliden}-4,4a,5,6,7,8-hexahydro-5-hydroxy-1,4a- | | |
| | | dimethylnaphthalen-2(3H)-one | | |
| 77 | 397.09 | (1S,8aS)-6-(4-Iodphenylamino)-1,2,3,4,6,7,8,8a-octahydro- | 3.08 | 397.07 |
| | | 5,8a-dimethylnaphthalen-1-ol | | |
| 78 | 448.20 | (3E,4aS,5S)-3-[4-(2-Methoxy-4-nitrophenylamino)benzyliden]- | 3.25 | 448.24 |
| | | 4,4a,5,6,7,8-hexahydro-5-hydroxy-1,4a-dimethylnaphthalen- | | |
| | | 2(3H)-one | | |
| 79 | 499.10 | (3E,4aS,5S)-3-[4-(4-Iodphenylamino)benzyliden]-4,4a,5,6,7,8- | 3.11 | 499.11 |
| | | hexahydro-5-hydroxy-1,4a-dimethylnaphthalen-2(3H)-one | | |
| 80 | 272.14 | (3E,4aS,5S)-3-[(Furan-3-yl)methylen]-4,4a,5,6,7,8-hexahydro- | 2.80 | 272.15 |
| | | 5-hydroxy-1,4a-dimethylnaphthalen-2(3H)-one | | |
| 81 | 360.07 | (3E,4aS,5S)-3-(3-Brombenzyliden)-4,4a,5,6,7,8-hexahydro-5- | 2.74 | 360.08 |
| | | hydroxy-1,4a-dimethylnaphthalen-2(3H)-one | | |
| 82 | 262.19 | (3E,4aS,5S)-4,4a,5,6,7,8-Hexahydro-5-hydroxy-1,4a- | 2.21 | 262.20 |
| | | dimethyl-3-(2-methylbutyliden)-naphthalen-2(3H)-one | | |
| 83 | 318.14 | (E,4aS,5S)-3-(4-Chlorbenzyliden)-octahydro-5-hydroxy-1,4a- | 2.80 | 318.14 |
| | | dimethylnaphthalen-2(1H)-one | | |
| 84 | 330.16 | (3E,4aS,5S)-3-(3-Fluor-4-methoxybenzyliden)-4,4a,5,6,7,8- | 2.93 | 330.14 |
| | | hexahydro-5-hydroxy-1,4a-dimethylnaphthalen-2(3H)-one | | |
| 85 | 300.15 | (3E,4aS,5S)-3-(4-Fluorbenzyliden)-4,4a,5,6,7,8-hexahydro-5- | 2.71 | 300.16 |
| | | hydroxy-1,4a-dimethylnaphthalen-2(3H)-one | | |
| 86 | 287.19 | (E,4aS,5S)-Octahydro-5-hydroxy-1,4a-dimethyl-3-[(1-methyl- | 2.78 | 287.18 |
| | | 1H-pyrrol-2-yl)methylen]naphthalen-2(1H)-one | | |
| 87 | 320.16 | (E,4aS,5S)-3-(3,4-Difluorbenzyliden)-octahydro-5-hydroxy- | 2.86 | 320.15 |
| | | 1,4a-dimethylnaphthalen-2(1H)-one | | |
| 88 | 336.13 | (E,4aS,5S)-3-(2-Chlor-6-fluorbenzyliden)-octahydro-5- | 2.94 | 334.15 |
| | | hydroxy-1,4a-dimethylnaphthalen-2(1H)-one | | |
| 89 | 326.22 | (E,4aS,5S)-3-(4-Ethyl-2-methylbenzyliden)-octahydro-5- | 2.64 | 326.22 |
| | | hydroxy-1,4a-dimethylnaphthalen-2(1H)-one | | |
| 90 | 432.30 | (3E,4aS,5S)-3-[4-(Undec-1-inyl)benzyliden]-4,4a,5,6,7,8- | 2.96 | 432.31 |
| | | hexahydro-5-hydroxy-1,4a-dimethylnaphthalen-2(3H)-one | | |
| 91 | 390.26 | (3E,4aS,5S)-3-[4-(Oct-1-inyl)benzyliden]-4,4a,5,6,7,8- | 2.84 | 390.24 |
| | | hexahydro-5-hydroxy-1,4a-dimethylnaphthalen-2(3H)-one | | |
| 92 | 474.35 | (3E,4aS,5S)-3-[4-(Tetradec-1-inyl)benzyliden]-4,4a,5,6,7,8- | 3.10 | 474.38 |
| | | hexahydro-5-hydroxy-1,4a-dimethylnaphthalen-2(3H)-one | | |
| 93 | 438.26 | (3E,4aS,5S)-3-{4-[2-(4-tert-Butylphenyl)ethinyl]benzyliden}- | 3.02 | 438.28 |
| | | 4,4a,5,6,7,8-hexahydro-5-hydroxy-1,4a-dimethylnaphthalen- | | |
| | | 2(3H)-one | | |
| 94 | 322.22 | (3E,4aS,5S)-3-[4-(3,3-Dimethylbut-1-inyl)benzyliden]-4,4a,5,6, | 2.67 | 322.24 |
| | | 7,8-hexahydro-5-hydroxy-1,4a-dimethylnaphthalen-2(3H)-one | | |
| 95 | 390.22 | (3E,4aS,5S)-3-{4-[2-(1-Hydroxycyclopentyl)ethinyl]- | 2.84 | 390.21 |
| | | benzyliden}-4,4a,5,6,7,8-hexahydro-5-hydroxy-1,4a- | | |
| | | dimethylnaphthalen-2(3H)-one | | |
| 96 | 192.15 | (1S,8aS)-1,2,3,4,6,7,8,8a-Octahydro-5,8a-dimethyl-6- | 2.02 | 192.16 |
| | | methylennaphthalen-1-ol | | |
| 97 | 280.18 | (1S,7E,8aS)-7-Benzyliden-1,2,3,4,6,7,8,8a-octahydro-5,8a- | 2.18 | 280.18 |
| | | dimethyl-6-methylennaphthalen-1-ol | | |
| 98 | 260.21 | (1S,7E,8aS)-1,2,3,4,6,7,8,8a-Octahydro-5,8a-dimethyl-6- | 2.24 | 260.20 |
| | | methylen-7-pentylidennaphthalen-1-ol | | |
| 99 | 264.17 | (2E)-Ethyl 2-[(4S,4aS)-2,3,4,4a,5,6-hexahydro-4-hydroxy- | 2.46 | 264.19 |
| | | 4a,8-dimethylnaphthalen-7(1H)-yliden]acetat | | |
| 100 | 262.23 | (1S,6E,8aS)-6-Hexyliden-1,2,3,4,6,7,8,8a-octahydro-5,8a- | 2.21 | 262.24 |
| | | dimethylnaphthalen-1-ol | | |
| 101 | 268.18 | (1S,6E,8aS)-6-Benzyliden-1,2,3,4,6,7,8,8a-octahydro-5,8a- | 2.19 | 268.20 |
| | | dimethylnaphthalen-1-ol | | |
| 102 | 344.24 | (1S,8aS)-5-(3,5-Dimethoxybenzyl)-decahydro-5,8a-dimethyl- | 2.56 | 344.23 |
| | | 6-methylennaphthalen-1-ol | | |
| 103 | 330.18 | (E,4aS,5S)-3-(4-Hydroxy-3-methoxybenzyliden)-octahydro-5- | 3.07 | 330.18 |
| | | hydroxy-1,4a-dimethylnaphthalen-2(1H)-one | | |
| 104 | 336.17 | (3E,4aS,5S)-3-[4-(3-Hydroxyprop-1-inyl)benzyliden]-4,4a,5,6, | 2.88 | 336.16 |
| | | 7,8-hexahydro-5-hydroxy-1,4a-dimethylnaphthalen-2(3H)-one | | |
| 105 | 416.15 | (3E,4aS,5S)-3-{4-[2-(4-Chlorphenyl)ethinyl]benzyliden}- | 3.05 | 416.12 |
| | | 4,4a,5,6,7,8-hexahydro-5-hydroxy-1,4a-dimethylnaphthalen- | | |
| | | 2(3H)-one | | |
| 106 | 396.21 | (3E,4aS,5S)-3-[4-(3-Phenylprop-1-inyl)benzyliden]-4,4a,5,6, | 2.78 | 396.20 |
| | | 7,8-hexahydro-5-hydroxy-1,4a-dimethylnaphthalen-2(3H)-one | | |
| 107 | 330.15 | (3E,4aS,5S)-3-(2,3,4-Trihydroxybenzyliden)-4,4a,5,6,7,8- | 3.49 | 330.15 |
| | | hexahydro-5-hydroxy-1,4a-dimethylnaphthalen-2(3H)-one | | |
| 108 | 332.16 | (E,4aS,5S)-3-(2,3,4-Trihydroxybenzyliden)-octahydro-5- | 3.46 | 332.16 |
| | | hydroxy-1,4a-dimethylnaphthalen-2(1H)-one | | |
| 109 | 316.13 | (3E,4aS,5S)-3-(2,3,4-Trihydroxybenzyliden)-4,4a,5,6,7,8- | 3.46 | 316.12 |
| | | hexahydro-5-hydroxy-4a-methylnaphthalen-2(3H)-one | | |
| 110 | 406.18 | (3E,4aS,5S)-3-(2,3,4-Trihydroxybenzyliden)-4a-benzyl-4,4a,5, | 3.58 | 406.16 |
| | | 6,7,8-hexahydro-5-hydroxy-1-methylnaphthalen-2(3H)-one | | |
| 111 | 264.17 | (3E,4aS,5S)-4,4a,5,6,7,8-Hexahydro-5-hydroxy-3-[(R)-3- | 2.89 | 264.16 |
| | | hydroxybutyliden]-1,4a-dimethylnaphthalen-2(3H)-one | | |
| 112 | 266.19 | (E,4aS,5S)-Octahydro-5-hydroxy-3-[(R)-3-hydroxybutyliden]- | 2.92 | 266.19 |
| | | 1,4a-dimethylnaphthalen-2(1H)-one | | |
| 113 | 250.16 | (3E,4aS,5S)-4,4a,5,6,7,8-Hexahydro-5-hydroxy-3-[(R)-3- | 2.90 | 250.16 |
| | | hydroxybutyliden]4a-methylnaphthalen-2(3H)-one | | |
| 114 | 340.20 | (3E,4aS,5S)-4a-Benzyl-4,4a,5,6,7,8-hexahydro-5-hydroxy-3- | 2.98 | 340.20 |
| | | [(R)-3-hydroxybutyliden]-1-methylnaphthalen-2(3H)-one | | |
| 115 | 321.17 | (3E,4aS,5S)-3-[(1H-Indol-3-yl)methylen]-4,4a,5,6,7,8- | 3.01 | 321.17 |
| | | hexahydro-5-hydroxy-1,4a-dimethylnaphthalen-2(3H)-one | | |
| 116 | 323.19 | (E,4aS,5S)-3-[(1H-Indol-3-yl)methylen]-octahydro-5-hydroxy- | 2.99 | 323.22 |
| | | 1,4a-dimethylnaphthalen-2(1H)-one | | |
| 117 | 307.16 | (3E,4aS,5S)-3-[(1H-Indol-3-yl)methylen]-4,4a,5,6,7,8- | 3.01 | 307.18 |
| | | hexahydro-5-hydroxy-4a-methylnaphthalen-2(3H)-one | | |
| 118 | 397.20 | (3E,4aS,5S)-3-[(1H-Indol-3-yl)methylen]-4a-benzyl-4,4a,5, | 3.10 | 397.22 |
| | | 6,7,8-hexahydro-5-hydroxy-1-methylnaphthalen-2(3H)-one | | |
| 119 | 323.19 | (E,5S,8aS)-2-[1H-Indol-3-yl)methylen]-octahydro-6-hydroxy- | 2.97 | 323.19 |
| | | 5,8a-dimethylnaphthalen-1(2H)-one | | |
| 120 | 246.16 | (3E,4aS,5S)-3-[(E)-But-2-enyliden]-4,4a,5,6,7,8-hexahydro-5- | 2.52 | 246.15 |
| | | hydroxy-1,4a-dimethylnaphthalen-2(3H)-one | | |
| 121 | 248.18 | (3E,4aS,5S)-3-[(E)-But-2-enyliden]-octahydro-5-hydroxy-1,4a- | 2.49 | 248.17 |
| | | dimethylnaphthalen-2(1H)-one | | |
| 122 | 232.15 | (3E,4aS,5S)-3-[(E)-But-2-enyliden]-4,4a,5,6,7,8-hexahydro-5- | 2.54 | 232.14 |
| | | hydroxy-4a-methylnaphthalen-2(3H)-one | | |
| 123 | 322.19 | (3E,4aS,5S)-4a-Benzyl-3-[(E)-but-2-enyliden]-4,4a,5,6,7,8- | 2.56 | 322.17 |
| | | hexahydro-5-hydroxy-1-methylnaphthalen-2(3H)-one | | |
| 124 | 248.18 | (2E,8aS)-2-[(E)-But-2-enyliden]-octahydro-6-hydroxy-5,8a- | 2.52 | 248.20 |
| | | dimethylnaphthalen-1(2H)-one | | |
| 125 | 392.27 | (E,4aS,5S)-3-[4-(Oct-1-inyl)-benzyliden]-octahydro-5-hydroxy- | 2.86 | 392.27 |
| | | 1,4a-dimethylnaphthalen-2(1H)-one | | |
| 126 | 466.29 | (3E,4aS,5S)-3-[4-(Oct-1-inyl)benzyliden]-4a-benzyl-4,4a,5,6, | 2.89 | 466.27 |
| | | 7,8-hexahydro-5-hydroxy-1-methylnaphthalen-2(3H)-one | | |
| 127 | 504.40 | (E,4aS,5S)-3-[4-(Hexadec-1-inyl)benzyliden]-octahydro-5- | 2.90 | 504.41 |
| | | hydroxy-1,4a-dimethylnaphthalen-2(1H)-one | | |
| 128 | 578.41 | (3E,4aS,5S)-3-[4-(Hexadec-1-inyl)benzyliden]-4a-benzyl-4,4a, | 2.94 | 578.42 |
| | | 5,6,7,8-hexahydro-5-hydroxy-1-methylnaphthalen-2(3H)-one | | |
| 129 | 350.22 | (E,4aS,5S)-3-[4-(Pent-1-inyl)benzyliden]-octahydro-5- | 2.71 | 350.23 |
| | | hydroxy-1,4a-dimethylnaphthalen-2(1H)-one | | |
| 130 | 424.24 | (3E,4aS,5S)-3-[4-(Pent-1-inyl)benzyliden]-4a-benzyl-4,4a,5, | 2.76 | 424.26 |
| | | 6,7,8-hexahydro-5-hydroxy-1-methylnaphthalen-2(3H)-one | | |
| 131 | 434.32 | (E,4aS,5S)-3-[4-(Undec-1-inyl)benzyliden]-octahydro-5- | 3.01 | 434.30 |
| | | hydroxy-1,4a-dimethylnaphthalen-2(1H)-one | | |
| 132 | 508.33 | (3E,4aS,5S)-3-[4-(Undec-1-inyl)benzyliden]-4a-benzyl-4,4a, | 3.02 | 508.30 |
| | | 5,6,7,8-hexahydro-5-hydroxy-1-methylnaphthalen-2(3H)-one | | |
| 133 | 476.37 | (E,4aS,5S)-3-[4-(Tetradec-1-inyl)benzyliden]-octahydro-5- | 3.18 | 476.39 |
| | | hydroxy-1,4a-dimethylnaphthalen-2(1H)-one | | |
| 134 | 550.38 | (3E,4aS,5S)-3-[4-(Tetradec-1-inyl)benzyliden]-4a-benzyl-4,4a, | 3.27 | 550.35 |
| | | 5,6,7,8-hexahydro-5-hydroxy-1-methylnaphthalen-2(3H)-one | | |
| 135 | 359.19 | (3E,4aS,5S)-4a-Benzyl-4,4a,5,6,7,8-hexahydro-5-hydroxy-1- | 2.79 | 359.19 |
| | | methyl-3-[(pyridin-4-yl)methylen]naphthalen-2(3H)-one | | |
| 136 | 368.18 | (3E,4aS,5S)-3-[4-(2-Phenylethinyl)benzyliden]-4,4a,5,6,7,8- | 2.68 | 368.17 |
| | | hexahydro-5-hydroxy-4a-methylnaphthalen-2(3H)-one | | |
| 137 | 364.19 | (3E,4aS,5S)-3-{4-[2-(Trimethylsilyl)ethinyl]benzyliden}-4,4a, | 2.56 | 364.18 |
| | | 5,6,7,8-hexahydro-5-hydroxy-4a-methylnaphthalen-2(3H)-one | | |
| 138 | 390.22 | (3E,4aS,5S)-3-{4-[2-(1-Hydroxycyclohexyl)ethinyl]benzyliden}- | 2.89 | 390.21 |
| | | 4,4a,5,6,7,8-hexahydro-5-hydroxy-4a-methylnaphthalen- | | |
| | | 2(3H)-one | | |
| 139 | 251.22 | (1S,8aS)-6-(Butylamino)-1,2,3,4,6,7,8,8a-octahydro-5,8a- | 2.74 | 251.22 |
| | | dimethylnaphthalen-1-ol | | |
| 140 | 277.24 | (1S,8aS)-6-(Cyclohexylamino)-1,2,3,4,6,7,8,8a-octahydro- | 2.69 | 277.25 |
| | | 5,8a-dimethylnaphthalen-1-ol | | |
| 141 | 491.17 | (1S,7E,8aS)-7-(4-Iodbenzyliden)-6-(cyclohexylamino)- | 3.11 | 491.17 |
| | | 1,2,3,4,6,7,8,8a-octahydro-5,8a-dimethylnaphthalen-1-ol | | |
| 142 | 397.09 | (1S,8aS)-6-(4-Iodphenylamino)-1,2,3,4,6,7,8,8a-octahydro- | 2.78 | 397.11 |
| | | 5,8a-dimethylnaphthalen-1-ol | | |
| 143 | 489.15 | (1S,8aS)-6-(4-Iodphenylamino)-5-benzyl-decahydro-5,8a- | 2.94 | 489.16 |
| | | dimethylnaphthalen-1-ol | | |
| 144 | 346.19 | (1S,8aS)-6-(2-Methoxy-4-nitrophenylamino)-1,2,3,4,6,7,8,8a- | 2.86 | 346.21 |
| | | octahydro-5,8a-dimethylnaphthalen-1-ol | | |
| 145 | 549.17 | (1S,8aS)-5-(3,5-Dimethoxybenzyl)-6-(4-Iodphenylamino)- | 3.31 | 549.14 |
| | | decahydro-5,8a-dimethylnaphthalen-1-ol | | |
| 146 | 429.32 | (1S,8aS)-5-(3,5-Dimethoxybenzyl)-6-(cyclohexylamino)- | 2.94 | 429.30 |
| | | decahydro-5,8a-dimethylnaphthalen-1-ol | | |
| 147 | 403.31 | (1S,8aS)-5-(3,5-Dimethoxybenzyl)-6-(butylamino)-decahydro- | 2.98 | 403.30 |
| | | 5,8a-dimethylnaphthalen-1-ol | | |
| 148 | 465.15 | (1S,8aS)-7-(4-Iodbenzyl)-6-(butylamino)-1,2,3,4,6,7,8,8a- | 3.04 | 465.17 |
| | | octahydro-5,8a-dimethylnaphthalen-1-ol | | |
| 149 | 406.25 | (4aS,5S)-3-[4-(3-tert-Butoxybut-1-inyl)benzyl]-4,4a,5,6,7,8- | 2.93 | 406.25 |
| | | hexahydro-5-hydroxy-1,4a-dimethylnaphthalen-2(3H)-one | | |
| 150 | 439.25 | (4aS,5S)-3-{4-[3-(N-Benzyl-N-methylamino)prop-1- | 3.07 | 439.24 |
| | | inyl]benzyl}-4,4a,5,6,7,8-hexahydro-5-hydroxy-1,4a- | | |
| | | dimethylnaphthalen-2(3H)-one | | |
| 151 | 383.19 | (4aS,5S)-{3-[4-(2-(Pyridin-2-yl)ethinyl]benzyl}4,4a,5,6,7,8- | 2.92 | 383.20 |
| | | hexahydro-5-hydroxy-1,4a-dimethylnaphthalen-2(3H)-one | | |
| 152 | 350.19 | (4aS,5S)-3-[4-(4-Hydroxybut-1-inyl)benzyl]-4,4a,5,6,7,8- | 3.18 | 250.19 |
| | | hexahydro-5-hydroxy-1,4a-dimethylnaphthalen-2(3H)-one | | |
| 153 | 282.18 | Ethyl-2-[(4aS,5S)-decahydro-5-hydroxy-1,4a-dimethyl-2- | 2.84 | 282.18 |
| | | oxonaphthalen-1-yl]acetat | | |
| 154 | 399.22 | (3E,4aS,5S)-3-[(9-Ethyl-9H-carbazol-3-yl)methylen]-4,4a,5,6, | 3.11 | 399.20 |
| | | 7,8-hexahydro-5-hydroxy-1,4a-dimethylnaphthalen-2(3H)-one | | |
| 155 | 452.24 | (E,4aS,5S)-1-(3,5-Dimethoxy-benzyl)-3-(4-fluorbenzyliden)- | 3.19 | 452.25 |
| | | octahydro-5-hydroxy-1,4a-dimethylnaphthalen-2(1H)-one | | |
| 156 | 494.27 | (E,4aS,5S)-3-(3,4-Dimethoxybenzyliden)-1-(3,5-dimethoxy- | 3.38 | 494.29 |
| | | benzyl)-octahydro-5-hydroxy-1,4a-dimethylnaphthalen-2(1H)- | | |
| | | one | | |
| 157 | 324.17 | (E,4aS,5S)-3-[(Benzofuran-2-yl)methylen]-octahydro-5- | 2.94 | 324.16 |
| | | hydroxy-1,4a-dimethylnaphthalen-2(1H)-one | | |
| 158 | 289.20 | (3E,4aS,5S)-Octahydro-3-[(4,5-dihydro-3-methyl-1H-pyrrol-2- | 2.81 | 289.20 |
| | | yl)methylen]-5-hydroxy-1,4a-dimethylnaphthalen-2(1H)-one | | |
| 159 | 372.19 | (3E,4aS,5S)-3-(2,4,5-Trimethoxybenzyliden)-4,4a,5,6,7,8- | 3.04 | 372.18 |
| | | hexahydro-5-hydroxy-1,4a-dimethylnaphthalen-2(3H)-one | | |
| 160 | 372.11 | (3E,4aS,5R)-4,4a,5,6,7,8-Hexahydro-5-hydroxy-1,4a- | 2.83 | 372.11 |
| | | dimethyl-3-(perfluor-benzyliden)naphthalen-2(3H)-one | | |
| 161 | 362.09 | (E,4aS,5S)-3-(4-Brombenzyliden)-octahydro-5-hydroxy-1,4a- | 2.71 | 362.09 |
| | | dimethylnaphthalen-2(1H)-one | | |
| 162 | 287.19 | (E,4aS,5S)-Octahydro-5-hydroxy-1,4a-dimethyl-3-[(1-methyl- | 2.64 | 287.19 |
| | | 1H-pyrrol-2-yl)methylen]naphthalen-2(1H)-one | | |
| 163 | 264.21 | (E,4aS,5S)-Octahydro-5-hydroxy-1,4a-dimethyl-3-(2-methyl- | 2.35 | 264.20 |
| | | butyliden)naphthalen-2(1H)-one | | |
| 164 | 302.17 | (E,4aS,5S)-3-(4-Fluorbenzyliden)-octahydro-5-hydroxy-1,4a- | 2.69 | 302.18 |
| | | dimethylnaphthalen-2(1H)-one | | |
| 165 | 408.06 | (3E,4R,4aS,5S)-3-(4-Iodbenzyliden)-4,4a,5,6,7,8-hexahydro- | 2.83 | 408.07 |
| | | 5-hydroxy-4,4a-dimethylnaphthalen-2(3H)-one | | |
| 166 | 316.12 | (3E,4R,4aS,5S)-3-(3-Chlorbenzyliden)-4,4a,5,6,7,8- | 2.75 | 316.12 |
| | | hexahydro-5-hydroxy-4,4a-dimethylnaphthalen-2(3H)-one | | |
| 167 | | 5-Hydroxy-1-methyl-7-phenyl-4,4a,5,6,7,8,-hexahydro-3H- | | |
| | | naphthalen-2-one | | |
| 168 | 360.07 | (3E,4aS,5S)-3-(3-Brombenzyliden)-4,4a,5,6,7,8-hexahydro-5- | 2.72 | 360.07 |
| | | hydroxy-1,4a-dimethylnaphthalen-2(3H)-one | | |
| 169 | 360.07 | (3E,4aS,5S)-3-(2-Brombenzyliden)-4,4a,5,6,7,8-hexahydro-5- | 2.80 | 360.08 |
| | | hydroxy-1,4a-dimethylnaphthalen-2(3H)-one | | |
| 170 | 220.15 | (3E,4aS,5S)-3-Ethyliden-4,4a,5,6,7,8-hexahydro-5-hydroxy- | 2.30 | 220.15 |
| | | 1,4a-dimethylnaphthalen-2(3H)-one | | |
| 171 | 374.21 | (E,4aS,5S)-3-(2,3,4-Trimethoxy-benzyliden)-octahydro-5- | 3.08 | 374.21 |
| | | hydroxy-1,4a-dimethylnaphthalen-2(1H)-one | | |
| 172 | 330.17 | (E,4aS,5S)-3-[4-(Methylthio)-benzyliden]-octahydro-5- | 2.88 | 330.17 |
| | | hydroxy-1,4a-dimethylnaphthalen-2(1H)-one | | |
| 173 | 328.24 | (3E,4aS,5S)-Octahydro-5-hydroxy-1,4a-dimethyl-3-{[(S)-4- | 2.69 | 328.25 |
| | | (prop-1-en-2-yl)cyclohex-1-enyl]-methylen}naphthalen-2(1H)- | | |
| | | one | | |
| 174 | 288.17 | (E,4aS,5S)-Octahydro-5-hydroxy-1,4a-dimethyl-3-[(5- | 2.74 | 288.16 |
| | | methylfuran-2-yl)methylen]naphthalen-2(1H)-one | | |
| 175 | 388.20 | (3E,4R,4aS,5S)-3-[2-(Benzyloxy)-benzyliden]-4,4a,5,6,7,8- | 2.98 | 388.20 |
| | | hexahydro-5-hydroxy-4,4a-dimethylnaphthalen-2(3H)-one | | |
| 176 | 272.14 | (3E,4R,4aS,5S)-3-[(Furan-3-yl)methylen]-4,4a,5,6,7,8- | 2.66 | 272.15 |
| | | hexahydro-5-hydroxy-4,4a-dimethylnaphthalen-2(3H)-one | | |
| 177 | 360.07 | (3E,4aS,5S)-3-(4-Brombenzyliden)-4,4a,5,6,7,8-hexahydro-5- | 2.74 | 360.07 |
| | | hydroxy-1,4a-dimethylnaphthalen-2(3H)-one | | |
| 178 | 262.20 | (3E,4aS,5S)-4,4a,5,6,7,8-Hexahydro-5-hydroxy-1,4a- | 2.31 | 262.20 |
| | | dimethyl-3-(2-methyl-butylidene)naphthalen-2(3H)-one | | |
| 179 | 300.15 | (3E,4aS,5S)-3-(4-Fluorbenzyliden)-4,4a,5,6,7,8-hexahydro-5- | 2.69 | 300.15 |
| | | hydroxy-1,4a-dimethylnaphthalen-2(3H)-one | | |
| 180 | 410.07 | (E,4aS,5S)-3-(4-Iodbenzyliden)-octahydro-5-hydroxy-1,4a- | 2.83 | 410.08 |
| | | dimethylnaphthalen-2(1H)-one | | |
| 181 | 264.21 | (E,4aS,5S)-Octahydro-5-hydroxy-1,4a-dimethyl-3-(2-methyl- | 2.34 | 264.22 |
| | | butyliden)naphthalen-2(1H)-one | | |
| 182 | 410.07 | (E,4aS,5S)-3-(2-Iodbenzyliden)-octahydro-5-hydroxy-1,4a- | 2.87 | 410.08 |
| | | dimethylnaphthalen-2(1H)-one | | |
| 183 | 318.14 | (E,4aS,5S)-3-(3-Chlorbenzyliden)-octahydro-5-hydroxy-1,4a- | 2.69 | 318.15 |
| | | dimethylnaphthalen-2(1H)-one | | |
| 184 | 300.15 | (3E,4R,4aS,5S)-3-(2-Fluorbenzyliden)-4,4a,5,6,7,8- | 2.71 | 300.16 |
| | | hexahydro-5-hydroxy-4,4a-dimethylnaphthalen-2(3H)-one | | |
| 185 | 258.13 | (3E,4aS,5S)-3-[(Furan-2-yl)methylen]-4,4a,5,6,7,8-hexahydro- | 2.74 | 258.13 |
| | | 5-hydroxy-4a-methylnaphthalen-2(3H)-one | | |
| 186 | 372.19 | (3E,4aS,5S)-3-(2,3,4-Trimethoxybenzyliden)-4,4a,5,6,7,8- | 3.04 | 372.20 |
| | | hexahydro-5-hydroxy-1,4a-dimethylnaphthalen-2(3H)-one | | |
| 187 | 328.15 | (3E,4aS,5S)-3[4-(Methylthio)benzyliden]-4,4a,5,6,7,8- | 2.88 | 328.13 |
| | | hexahydro-5-hydroxy-1,4a-dimethylnaphthalen-2(3H)-one | | |
| 188 | 326.22 | (3E,4aS,5S)-4,4a,5,6,7,8-Hexahydro-5-hydroxy-1,4a- | 2.61 | 326.22 |
| | | dimethyl-3-{[(S)-4-(prop-1-en-2-yl)cyclohex-1-enyl]methylen}- | | |
| | | naphthalen-2(3H)-one | | |
| 189 | 286.16 | (3E,4aS,5S)-4,4a,5,6,7,8-Hexahydro-5-hydroxy-1,4a- | 2.72 | 286.14 |
| | | dimethyl-3-[(5-methylfuran-2-yl)methylen]naphthalen-2(3H)-on | | |
| 190 | 390.22 | (E,4aS,5S)-3-[2-(Benzyloxy)-benzyliden]-octahydro-5- | 2.99 | 390.22 |
| | | hydroxy-1,4a-dimethylnaphthalen-2(1H)-one | | |
| 191 | 408.06 | (3E,4R,4aS,5S)-3-(3-Iodbenzyliden)-4,4a,5,6,7,8-hexahydro- | 2.85 | 408.07 |
| | | 5-hydroxy-4,4a-dimethylnaphthalen-2(3H)-one | | |
| 192 | 408.06 | (3E,4aS,5S)-3-(4-Iodbenzyliden)-4,4a,5,6,7,8-hexahydro-5- | 2.79 | 408.04 |
| | | hydroxy-1,4a-dimethylnaphthalen-2(3H)-one | | |
| 193 | 262.19 | (3E,4aS,5S)-4,4a,5,6,7,8-Hexahydro-5-hydroxy-1,4a- | 2.29 | 262.18 |
| | | dimethyl-3-(2-methyl-butyliden)naphthalen-2(3H)-one | | |
| 194 | 408.06 | (3E,4aS,5S)-3-(2-Iodbenzyliden)-4,4a,5,6,7,8-hexahydro-5- | 2.87 | 408.06 |
| | | hydroxy-1,4a-dimethylnaphthalen-2(3H)-one | | |
| 195 | 316.12 | (3E,4aS,5S)-3-(3-Chlorbenzyliden)-4,4a,5,6,7,8-hexahydro-5- | 2.74 | 316.13 |
| | | hydroxy-1,4a-dimethylnaphthalen-2(3H)-one | | |
| 196 | 302.17 | (E,4aS,5S)-3-(2-Fluorbenzyliden)-octahydro-5-hydroxy-1,4a- | 2.80 | 302.16 |
| | | dimethylnaphthalen-2(1H)-one | | |
| 197 | 372.19 | (3E,4R,4aS,5S)-3-(2,4,5-Trimethoxybenzyliden)-4,4a,5,6,7,8- | 3.11 | 372.19 |
| | | hexahydro-5-hydroxy-4,4a-dimethylnaphthalen-2(3H)-one | | |
| 198 | 346.06 | (3E,4aS,5S)-3-(4-Brombenzyliden)-4,4a,5,6,7,8-hexahydro-5- | 2.83 | 346.07 |
| | | hydroxy-4a-methylnaphthalen-2(3H)-one | | |
| 199 | 388.20 | (3E,4aS,5S)-3-[2-(Benzyloxy)-benzyliden]-4,4a,5,6,7,8- | 3.02 | 388.20 |
| | | hexahydro-5-hydroxy-1,4a-dimethylnaphthalen-2(3H)-one | | |
| 200 | 326.22 | (3E,4aS,5S)-4,4a,5,6,7,8-Hexahydro-5-hydroxy-1,4a- | 2.88 | 326.21 |
| | | dimethyl-3-[(2,6,6-trimethylcyclohexa-1,3-dienyl)- | | |
| | | methylen]naphthalen-2(3H)-one | | |
| 201 | 354.26 | (E,4aS,5S)-3-(2,3,4,5,6-Pentamethylbenzyliden)-octahydro-5- | 2.81 | 354.25 |
| | | hydroxy-1,4a-dimethylnaphthalen-2(1H)-on | | |
| 202 | 410.07 | (E,4aS,5S)-3-(3-Iodbenzyliden)-octahydro-5-hydroxy-1,4a- | 2.81 | 410.07 |
| | | dimethylnaphthalen-2(1H)-one | | |
| 203 | 282.16 | (3E,4R,4aS,5S)-3-Benzyliden-4,4a,5,6,7,8-hexahydro-5- | 2.40 | 282.15 |
| | | hydroxy-4,4a-dimethylnaphthalen-2(3H)-one | | |
| 204 | 360.07 | (3E,4R,4aS,5S)-3-(3-Brombenzyliden)-4,4a,5,6,7,8- | 2.75 | 360.09 |
| | | hexahydro-5-hydroxy-4,4a-dimethylnaphthalen-2(3H)-one | | |
| 205 | 360.07 | (3E,4R,4aS,5S)-3-(2-Brombenzyliden)-4,4a,5,6,7,8- | 2.79 | 360.08 |
| | | hexahydro-5-hydroxy-4,4a-dimethylnaphthalen-2(3H)-one | | |
| 206 | 220.15 | (3E,4R,4aS,5S)-3-Ethyliden-4,4a,5,6,7,8-hexahydro-5- | 2.29 | 220.15 |
| | | hydroxy-4,4a-dimethylnaphthalen-2(3H)-one | | |
| 207 | 374.21 | (E,4aS,5S)-3-(2,3,4-Trimethoxy-benzyliden)-octahydro-5- | 3.08 | 374.20 |
| | | hydroxy-1,4a-dimethylnaphthalen-2(1H)-one | | |
| 208 | 300.15 | (3E,4aS,5S)-3-(2-Fluorbenzyliden)-4,4a,5,6,7,8-hexahydro-5- | 2.81 | 300.16 |
| | | hydroxy-1,4a-dimethylnaphthalen-2(3H)-one | | |
| 209 | 276.21 | (3E,4aS,5S)-4,4a,5,6,7,8-Hexahydro-5-hydroxy-1,4a- | 2.75 | 276.20 |
| | | dimethyl-3-(2-methylpentyliden)-naphthalen-2(3H)-one | | |
| 210 | 374.21 | (E,4aS,5S)-3-(2,4,5-Trimethoxybenzyliden)-octahydro-5- | 3.03 | 374.19 |
| | | hydroxy-1,4a-dimethylnaphthalen-2(1H)-one | | |
| 211 | 374.13 | (E,4aS,5S)-Octahydro-5-hydroxy-1,4a-dimethyl-3-(perfluor- | 2.84 | 374.14 |
| | | benzyliden)naphthalen-2(1H)-one | | |
| 212 | 360.07 | (3E,4R,4aS,5S)-3-(4-Brombenzyliden)-4,4a,5,6,7,8- | 2.81 | 360.09 |
| | | hexahydro-5-hydroxy-4,4a-dimethylnaphthalen-2(3H)-one | | |
| 213 | 300.15 | (3E,4R,4aS,5S)-3-(4-Fluorbenzyliden)-4,4a,5,6,7,8- | 2.76 | 300.15 |
| | | hexahydro-5-hydroxy-4,4a-dimethylnaphthalen-2(3H)-one | | |
| 214 | 394.04 | (3E,4aS,5S)-3-(4-Iodbenzyliden)-4,4a,5,6,7,8-hexahydro-5- | 2.89 | 394.05 |
| | | hydroxy-4a-methylnaphthalen-2(3H)-one | | |
| 215 | 352.24 | (3E,4aS,5S)-3-(2,3,4,5,6-Pentamethylbenzyliden)-4,4a,5,6, | 2.74 | 352.24 |
| | | 7,8-hexahydro-5-hydroxy-1,4a-dimethylnaphthalen-2(3H)-one | | |
| 216 | 408.06 | (3E,4aS,5S)-3-(3-Iodbenzyliden)-4,4a,5,6,7,8-hexahydro-5- | 2.84 | 408.07 |
| | | hydroxy-1,4a-dimethylnaphthalen-2(3H)-one | | |
| 217 | 300.15 | (3E,4aS,5S)-3-(3-Fluorbenzyliden)-4,4a,5,6,7,8-hexahydro-5- | 2.71 | 300.15 |
| | | hydroxy-1,4a-dimethylnaphthalen-2(3H)-one | | |
| 218 | 362.09 | (E,4aS,5S)-3-(3-Brombenzyliden)-octahydro-5-hydroxy-1,4a- | 2.79 | 362.09 |
| | | dimethylnaphthalen-2(1H)-one | | |
| 219 | 362.09 | (E,4aS,5S)-3-(2-Brombenzyliden)-octahydro-5-hydroxy-1,4a- | 2.83 | 362.09 |
| | | dimethylnaphthalen-2(1H)-one | | |
| 220 | 222.16 | (E,4aS,5S)-3-Ethyliden-octahydro-5-hydroxy-1,4a- | 2.34 | 222.17 |
| | | dimethylnaphthalen-2(1H)-one | | |
| 221 | 372.19 | (3E,4R,4aS,5S)-3-(2,3,4-Trimethoxybenzyliden)-4,4a,5,6,7,8- | 3.10 | 372.19 |
| | | hexahydro-5-hydroxy-4,4a-dimethylnaphthalen-2(3H)-one | | |
| 222 | 328.15 | (3E,4R,4aS,5S)-3-[4-(Methylthio)benzyliden]-4,4a,5,6,7,8- | 2.79 | 328.14 |
| | | hexahydro-5-hydroxy-4,4a-dimethylnaphthalen-2(3H)-one | | |
| 223 | 374.19 | (3E,4aS,5S)-3-[2-(Benzyloxy)benzyliden]-4,4a,5,6,7,8- | 2.98 | 374.19 |
| | | hexahydro-5-hydroxy-4a-methylnaphthalen-2(3H)-one | | |
| 224 | 286.14 | (3E,4aS,5S)-3-(2-Fluorbenzyliden)-4,4a,5,6,7,8-hexahydro-5- | 2.77 | 286.14 |
| | | hydroxy-4a-methylnaphthalen-2(3H)-one | | |
| 225 | 262.19 | (3E,4aS,5S)-4,4a,5,6,7,8-Hexahydro-5-hydroxy-4a-methyl-3- | 2.46 | 262.19 |
| | | (2-methylpentyliden)naphthalen-2(3H)-one | | |
| 226 | 312.14 | 4-{(1E)-[(4S,4aS)-1,2,3,4,4a,5-Hexahydro-4-hydroxy-4a- | 3.24 | 312.15 |
| | | methyl-7-oxonaphthalen-6(7H)-yliden]methyl}benzoic acid | | |
| 227 | 348.17 | (3E,4aS,5S)-4a-Benzyl-3-[(furan-2-yl)methylen]-4,4a,5,6,7,8- | 2.94 | 348.18 |
| | | hexahydro-5-hydroxy-1-methylnaphthalen-2(3H)-one | | |
| 228 | 448.22 | (3E,4aS,5S)-3-(2,4,5-Trimethoxy-benzyliden)-4a-benzyl-4,4a, | 3.28 | 448.22 |
| | | 5,6,7,8-hexahydro-5-hydroxy-1-methylnaphthalen-2(3H)-one | | |
| 229 | 448.15 | (3E,4aS,5S)-4a-Benzyl-4,4a,5,6,7,8-hexahydro-5-hydroxy-1- | 3.04 | 448.16 |
| | | methyl-3-(perfluorbenzyliden)naphthalen-2(3H)-one | | |
| 230 | 386.09 | (3E,4R,4aS,5S)-3-(4-Brombenzyliden)-4a-allyl-4,4a,5,6,7,8- | 3.10 | 386.09 |
| | | hexahydro-5-hydroxy-4-methylnaphthalen-2(3H)-one | | |
| 231 | 311.19 | (3E,4R,4aS,5S)-4a-Allyl-4,4a,5,6,7,8-hexahydro-5-hydroxy-4- | 3.00 | 311.20 |
| | | methyl-3-[(1-methyl-1H-pyrrol-2-yl)methylen]naphthalen- | | |
| | | 2(3H)-one | | |
| 232 | 288.21 | (3E,4R,4aS,5S)-4a-Allyl-4,4a,5,6,7,8-hexahydro-5-hydroxy-4- | 2.74 | 288.21 |
| | | methyl-3-(2-methylbutylidene)-naphthalen-2(3H)-one | | |
| 233 | 396.06 | (E,5S)-2-(4-Iodbenzyliden)-octahydro-6-hydroxy-5- | 2.86 | 396.07 |
| | | methylnaphthalen-1(2H)-one | | |
| 234 | 394.04 | (3E,4aS,5S)-3-(3-Iodbenzyliden)-4,4a,5,6,7,8-hexahydro-5- | 2.79 | 394.03 |
| | | hydroxy-4a-methylnaphthalen-2(3H)-one | | |
| 235 | 286.14 | (3E,4aS,5S)-3-(3-Fluorbenzyliden)-4,4a,5,6,7,8-hexahydro-5- | 2.66 | 286.14 |
| | | hydroxy-4a-methylnaphthalen-2(3H)-one | | |
| 236 | 358.18 | (3E,4aS,5S)-4a-Benzyl-3-benzyliden-4,4a,5,6,7,8-hexahydro- | 2.84 | 358.18 |
| | | 5-hydroxy-1-methylnaphthalen-2(3H)-one | | |
| 237 | 436.10 | (3E,4aS,5S)-3-(3-Brombenzyliden)-4a-benzyl-4,4a,5,6,7,8- | 3.03 | 436.10 |
| | | hexahydro-5-hydroxy-1-methylnaphthalen-2(3H)-one | | |
| 238 | 296.18 | (3E,4aS,5S)-4a-Benzyl-3-ethyliden-4,4a,5,6,7,8-hexahydro-5- | 2.65 | 286.18 |
| | | hydroxy-1-methylnaphthalen-2(3H)-one | | |
| 239 | 398.21 | (3E,4R,4aS,5S)-3-(2,3,4-Trimethoxybenzyliden)-4a-allyl-4, | 3.24 | 398.21 |
| | | 4a,5,6,7,8-hexahydro-5-hydroxy-4-methylnaphthalen-2(3H)- | | |
| | | one | | |
| 240 | 354.17 | (3E,4R,4aS,5S)-3-[4-(Methylthio)-benzyliden]-4a-allyl-4,4a,5, | 3.06 | 354.16 |
| | | 6,7,8-hexahydro-5-hydroxy-4-methylnaphthalen-2(3H)-one | | |
| 241 | 352.24 | (3E,4R,4aS,5S)-4a-Allyl-4,4a,5,6,7,8-hexahydro-5-hydroxy-4- | 2.98 | 352.24 |
| | | methyl-3-{[(S)-4-(prop-1-en-2-yl)cyclohex-1-enyl]methylen}- | | |
| | | naphthalen-2(3H)-one | | |
| 242 | 312.17 | (3E,4R,4aS,5S)-4a-Allyl-4,4a,5,6,7,8-hexahydro-5-hydroxy-4- | 3.14 | 312.18 |
| | | methyl-3-[(5-methylfuran-2-yl)methylen]naphthalen-2(3H)-on | | |
| 243 | 390.22 | (E,5S,8aS)-2-(2-(Benzyloxy)-benzyliden)-octahydro-6- | 2.86 | 390.22 |
| | | hydroxy-5,8a-dimethylnaphthalen-1(2H)-one | | |
| 244 | 358.18 | (3E,4aS,5S)-3-(2,4,5-Trimethoxybenzyliden)-4,4a,5,6,7,8- | 3.01 | 358.18 |
| | | hexahydro-5-hydroxy-4a-methylnaphthalen-2(3H)-one | | |
| 245 | 358.10 | (3E,4aS,5S)-4,4a,5,6,7,8-Hexahydro-5-hydroxy-4a-methyl-3- | 2.89 | 358.09 |
| | | (perfluorbenzyliden)naphthalen-2(3H)-one | | |
| 246 | 338.22 | (3E,4aS,5S)-4a-Benzyl-4,4a,5,6,7,8-hexahydro-5-hydroxy-1- | 2.69 | 338.22 |
| | | methyl-3-(2-methylbutyliden)naphthalen-2(3H)-one | | |
| 247 | 376.18 | (3E,4aS,5S)-3-(4-Fluorbenzyliden)-4a-benzyl-4,4a,5,6,7,8- | 2.87 | 376.18 |
| | | hexahydro-5-hydroxy-1-methylnaphthalen-2(3H)-one | | |
| 248 | 334.07 | (3E,4R,4aS,5S)-3-(4-Iodbenzyliden)-4a-allyl-4,4a,5,6,7,8- | 3.02 | 334.06 |
| | | hexahydro-5-hydroxy-4-methylnaphthalen-2(3H)-one | | |
| 249 | 288.21 | (3E,4R,4aS,5S)-4a-Allyl-4,4a,5,6,7,8-hexahydro-5-hydroxy-4- | 2.68 | 288.20 |
| | | methyl-3-(2-methylbutyliden)-naphthalen-2(3H)-one | | |
| 250 | 434.07 | (3E,4R,4aS,5S)-3-(2-Iodbenzyliden)-4a-allyl-4,4a,5,6,7,8- | 3.05 | 434.06 |
| | | hexahydro-5-hydroxy-4-methylnaphthalen-2(3H)-one | | |
| 251 | 302.17 | (E,5S,8aS)-2-(2-Fluorbenzyliden)-octahydro-6-hydroxy-5,8a- | 2.76 | 302.19 |
| | | dimethylnaphthalen-1(2H)-one | | |
| 252 | 346.06 | (3E,4aS,5S)-3-(3-Brombenzyliden)-4,4a,5,6,7,8-hexahydro-5- | 2.79 | 346.06 |
| | | hydroxy-4a-methylnaphthalen-2(3H)-one | | |
| 253 | 206.13 | (3E,4aS,5S)-3-Ethyliden-4,4a,5,6,7,8-hexahydro-5-hydroxy- | 2.24 | 206.13 |
| | | 4a-methylnaphthalen-2(3H)-one | | |
| 254 | 448.22 | (3E,4aS,5S)-3-(2,3,4-Trimethoxybenzyliden)-4a-benzyl- | 3.31 | 448.21 |
| | | 4,4a,5,6,7,8-hexahydro-5-hydroxy-1-methylnaphthalen-2(3H)- | | |
| | | one | | |
| 255 | 414.22 | (3E,4R,4aS,5S)-3-[2-(Benzyloxy)-benzyliden]-4a-allyl-4,4a,5, | 3.15 | 414.22 |
| | | 6,7,8-hexahydro-5-hydroxy-4-methylnaphthalen-2(3H)-one | | |
| 256 | 352.24 | (3E,4R,4aS,5S)-4a-Allyl-4,4a,5,6,7,8-hexahydro-5-hydroxy-4- | 2.97 | 352.24 |
| | | methyl-3-{[(R)-4-(prop-1-en-2-yl)cyclohex-1-enyl]- | | |
| | | methylen}naphthalen-2(3H)-one | | |
| 257 | 298.16 | (3E,4R,4aS,5S)-4a-Allyl-3-[(furan-3-yl)methylen]-4,4a,5,6,7,8- | 3.06 | 298.18 |
| | | hexahydro-5-hydroxy-4-methylnaphthalen-2(3H)-one | | |
| 258 | 352.24 | (3E,4R,4aS,5S)-4a-Allyl-4,4a,5,6,7,8-hexahydro-5-hydroxy-4- | 2.84 | 352.24 |
| | | methyl-3-[(2,6,6-trimethyl-cyclohexa-1,3-dienyl)methylen]- | | |
| | | naphthalen-2(3H)-one | | |
| 259 | 354.26 | (E,5S,8aS)-2-(2,3,4,5,6-Pentamethyl-benzyliden)-octahydro- | 2.76 | 354.26 |
| | | 6-hydroxy-5,8a-dimethylnaphthalen-1(2H)-one | | |
| 260 | 271.16 | (3E,4aS,5S)-4,4a,5,6,7,8-Hexahydro-5-hydroxy-4a-methyl-3- | 2.84 | 271.16 |
| | | [(1-methyl-1H-pyrrol-2-yl)methylen]naphthalen-2(3H)-one | | |
| 261 | 248.18 | (3E,4aS,5S)-4,4a,5,6,7,8-Hexahydro-5-hydroxy-4a-methyl-3- | 2.40 | 248.19 |
| | | (2-methylbutyliden)naphthalen-2(3H)-one | | |
| 262 | 286.14 | (3E,4aS,5S)-3-(4-Fluorbenzyliden)-4,4a,5,6,7,8-hexahydro-5- | 2.68 | 286.12 |
| | | hydroxy-4a-methylnaphthalen-2(3H)-one | | |
| 263 | 338.22 | (3E,4aS,5S)-4a-Benzyl-4,4a,5,6,7,8-hexahydro-5-hydroxy-1- | 2.76 | 338.22 |
| | | methyl-3-(2-methylbutyliden)naphthalen-2(3H)-one | | |
| 264 | 326.17 | (3E,4R,4aS,5S)-3-(2-Fluorbenzyliden)-4a-allyl-4,4a,5,6,7,8- | 2.88 | 326.17 |
| | | hexahydro-5-hydroxy-4-methylnaphthalen-2(3H)-one | | |
| 265 | 302.22 | (3E,4R,4aS,5S)-4a-Allyl-4,4a,5,6,7,8-hexahydro-5-hydroxy-4- | 2.75 | 302.21 |
| | | methyl-3-(2-methylpentyliden)-naphthalen-2(3H)-one | | |
| 266 | 274.16 | (E,5S,8aS)-2-[(Furan-2-yl)methylen]-octahydro-6-hydroxy- | 2.78 | 274.17 |
| | | 5,8a-dimethylnaphthalen-1(2H)-one | | |
| 267 | 374.21 | (E,5S,8aS)-2-(2,4,5-Trimethoxy-benzyliden)-octahydro-6- | 2.96 | 374.22 |
| | | hydroxy-5,8a-dimethylnaphthalen-1(2H)-one | | |
| 268 | 314.13 | (3E,4aS,5S)-3-[4-(Methylthio)-benzyliden]-4,4a,5,6,7,8- | 2.76 | 314.14 |
| | | hexahydro-5-hydroxy-4a-methylnaphthalen-2(3H)-one | | |
| 269 | 312.21 | (3E,4aS,5S)-4,4a,5,6,7,8-Hexahydro-5-hydroxy-4a-methyl-3- | 2.70 | 312.19 |
| | | {[(S)-4-(prop-1-en-2-yl)cyclohex-1-enyl]methylen}naphthalen- | | |
| | | 2(3H)-one | | |
| 270 | 272.14 | (3E,4aS,5S)-4,4a,5,6,7,8-Hexahydro-5-hydroxy-4a-methyl-3- | 2.73 | 272.14 |
| | | [(5-methylfuran-2-yl)methylen]naphthalen-2(3H)-one | | |
| 271 | 464.24 | (3E,4aS,5S)-4,4a,5,6,7,8-Hexahydro-5-hydroxy-4a-methyl-3- | 3.04 | 464.24 |
| | | [(5-methylfuran-2-yl)methylen]naphthalen-2(3H)-one | | |
| 272 | 402.26 | (3E,4aS,5S)-4a-Benzyl-4,4a,5,6,7,8-hexahydro-5-hydroxy-1- | 2.94 | 402.26 |
| | | methyl-3-{[(S)-4-(prop-1-en-2-yl)cyclohex-1- | | |
| | | enyl]methylen}naphthalen-2(3H)-one | | |
| 273 | 378.26 | (3E,4R,4aS,5S)-3-(2,3,4,5,6-Pentamethylbenzyliden)-4a-allyl- | 2.97 | 378.27 |
| | | 4,4a,5,6,7,8-hexahydro-5-hydroxy-4-methylnaphthalen-2(3H)- | | |
| | | one | | |
| 274 | 434.07 | (3E,4R,4aS,5S)-3-(3-Iodbenzyliden)-4a-allyl-4,4a,5,6,7,8- | 3.08 | 434.08 |
| | | hexahydro-5-hydroxy-4-methylnaphthalen-2(3H)-one | | |
| 275 | 326.17 | (3E,4R,4aS,5S)-3-(3-Fluorbenzyliden)-4a-allyl-4,4a,5,6,7,8- | 3.01 | 326.19 |
| | | hexahydro-5-hydroxy-4-methylnaphthalen-2(3H)-one | | |
| 276 | 362.09 | (E,5S,8aS)-2-(3-Brombenzyliden)-octahydro-6-hydroxy-5,8a- | 2.78 | 362.09 |
| | | dimethylnaphthalen-1(2H)-one | | |
| 277 | 248.18 | (3E,4aS,5S)-4,4a,5,6,7,8-Hexahydro-5-hydroxy-4a-methyl-3- | 2.24 | 248.17 |
| | | (2-methylbutyliden)naphthalen-2(3H)-one | | |
| 278 | 394.04 | (3E,4aS,5S)-3-(2-Iodbenzyliden)-4,4a,5,6,7,8-hexahydro-5- | 2.87 | 394.04 |
| | | hydroxy-4a-methylnaphthalen-2(3H)-one | | |
| 279 | 376.18 | (3E,4aS,5S)-3-(2-Fluorbenzyliden)-4a-benzyl-4,4a,5,6,7,8- | 2.95 | 376.18 |
| | | hexahydro-5-hydroxy-1-methylnaphthalen-2(3H)-one | | |
| 280 | 352.24 | (3E,4aS,5S)-4a-Benzyl-4,4a,5,6,7,8-hexahydro-5-hydroxy-1- | 2.78 | 352.23 |
| | | methyl-3-(2-methylpentyliden)naphthalen-2(3H)-one | | |
| 281 | 398.21 | (3E,4R,4aS,5S)-3-(2,4,5-Trimethoxybenzyliden)-4a-allyl-4,4a, | 3.22 | 334.13 |
| | | 5,6,7,8-hexahydro-5-hydroxy-4-methylnaphthalen-2(3H)-one | | |
| 282 | 260.18 | (3E,4R,4aS,5S)-4a-Allyl-4,4a,5,6,7,8-hexahydro-5-hydroxy-4- | 2.67 | 260.18 |
| | | methyl-3-propylidennaphthalen-2(3H)-one | | |
| 283 | 398.13 | (3E,4R,4aS,5S)-4a-Allyl-4,4a,5,6,7,8-hexahydro-5-hydroxy-4- | 2.89 | 398.12 |
| | | methyl-3-(perfluorbenzyliden)-naphthalen-2(3H)-one | | |
| 284 | 362.09 | (E,5S,8aS)-2-(4-Brombenzyliden)-octahydro-6-hydroxy-5,8a- | 2.77 | 362.09 |
| | | dimethylnaphthalen-1(2H)-one | | |
| 285 | 287.19 | (E,5S,8aS)-Octahydro-6-hydroxy-5,8a-dimethyl-2-[(1-methyl- | 2.84 | 287.20 |
| | | 1H-pyrrol-2-yl)methylen]naphthalen-1(2H)-one | | |
| 286 | 312.21 | (3E,4aS,5S)-4,4a,5,6,7,8-Hexahydro-5-hydroxy-4a-methyl-3- | 2.69 | 312.21 |
| | | {[(R)-4-(prop-1-en-2-yl)cyclohex-1-enyl]methylen}naphthalen- | | |
| | | 2(3H)-one | | |
| 287 | 258.13 | (3E,4aS,5S)-3-[(Furan-3-yl)methylen]-4,4a,5,6,7,8-hexahydro- | 2.75 | 258.11 |
| | | 5-hydroxy-4a-methylnaphthalen-2(3H)-one | | |
| 288 | 376.18 | (3E,4aS,5S)-3-(3-Fluorbenzyliden)-4a-benzyl-4,4a,5,6,7,8- | 2.91 | 376.18 |
| | | hexahydro-5-hydroxy-1-methylnaphthalen-2(3H)-one | | |
| 289 | 308.18 | (3E,4R,4aS,5S)-4a-Allyl-3-benzyliden-4,4a,5,6,7,8- | 2.83 | 308.17 |
| | | hexahydro-5-hydroxy-4-methylnaphthalen-2(3H)-one | | |
| 290 | 374.21 | (E,5S,8aS)-2-(2,3,4-Trimethoxy-benzyliden)-octahydro-6- | 2.96 | 374.21 |
| | | hydroxy-5,8a-dimethylnaphthalen-1(2H)-one | | |
| 291 | 330.17 | (E,5S,8aS)-2-(4-(Methylthio)-benzyliden)-octahydro-6- | 2.74 | 330.17 |
| | | hydroxy-5,8a-dimethylnaphthalen-1(2H)-one | | |
| 292 | 264.21 | (E,5S,8aS)-Octahydro-6-hydroxy-5,8a-dimethyl-2-(2-methyl- | 2.31 | 264.20 |
| | | butyliden)naphthalen-1(2H)-one | | |
| 293 | 410.07 | (E,5S,8aS)-2-(2-Iodbenzyliden)-octahydro-6-hydroxy-5,8a- | 2.82 | 410.09 |
| | | dimethylnaphthalen-1(2H)-one | | |
| 294 | 318.14 | (E,5S,8aS)-2-(3-Chlorbenzyliden)-octahydro-6-hydroxy-5,8a- | 2.74 | 318.15 |
| | | dimethylnaphthalen-1(2H)-one | | |
| 295 | 328.24 | (2E,5S,8aS)-Octahydro-6-hydroxy-5,8a-dimethyl-2-{[(S)-4- | 2.68 | 328.24 |
| | | (prop-1-en-2-yl)cyclohex-1-enyl]methylen}naphthalen-1(2H)- | | |
| | | one | | |
| 296 | 274.16 | (E,5S,8aS)-2-[(Furan-2-yl)methylen]-octahydro-6-hydroxy- | 2.72 | 274.16 |
| | | 5,8a-dimethylnaphthalen-1(2H)-one | | |
| 297 | 328.24 | (2E,5S,8aS)-Octahydro-6-hydroxy-5,8a-dimethyl-2-[(2,6,6- | 2.46 | 328.23 |
| | | trimethylcyclohexa-1,3-dienyl)-methylen]naphthalen-1(2H)-on | | |
| 298 | 278.22 | (E,5S,8aS)-Octahydro-6-hydroxy-5,8a-dimethyl-2-(2-methyl- | 2.28 | 278.22 |
| | | pentyliden)naphthalen-1(2H)-one | | |
| 299 | 328.17 | 4-{(E)-[(1S,4aS)-Octahydro-2-hydroxy-1,4a-dimethyl-5-oxo- | 2.94 | 328.15 |
| | | naphthalen-6(1H)-yliden]methyl}benzoic acid | | |
| 300 | 410.07 | (E,5S,8aS)-2-(3-Iodbenzyliden)-octahydro-6-hydroxy-5,8a- | 2.78 | 410.06 |
| | | dimethylnaphthalen-1(2H)-one | | |
| 301 | 302.17 | (E,5S,8aS)-2-(3-Fluorbenzyliden)-octahydro-6-hydroxy-5,8a- | 2.69 | 302.18 |
| | | dimethylnaphthalen-1(2H)-one | | |
| 302 | 236.18 | (E,5S,8aS)-Octahydro-6-hydroxy-5,8a-dimethyl-2-propyliden- | 2.27 | 236.19 |
| | | naphthalen-1(2H)-one | | |
| 303 | 374.13 | (E,5S,8aS)-Octahydro-6-hydroxy-5,8a-dimethyl-2-(perfluor- | 2.51 | 374.12 |
| | | benzyliden)naphthalen-1(2H)-one | | |
| 304 | 362.09 | (E,5S,8aS)-2-(2-Brombenzyliden)-octahydro-6-hydroxy-5,8a- | 2.76 | 362.09 |
| | | dimethylnaphthalen-1(2H)-one | | |
| 305 | 222.16 | (E,5S,8aS)-2-Ethyliden-octahydro-6-hydroxy-5,8a- | 2.24 | 222.15 |
| | | dimethylnaphthalen-1(2H)-one | | |
| 306 | 264.21 | (E,5S,8aS)-Octahydro-6-hydroxy-5,8a-dimethyl-2-(2-methyl- | 2.32 | 264.22 |
| | | butyliden)naphthalen-1(2H)-one | | |
| 307 | 302.17 | (E,5S,8aS)-2-(4-Fluorbenzyliden)-octahydro-6-hydroxy-5,8a- | 2.65 | 302.17 |
| | | dimethylnaphthalen-1(2H)-one | | |
| 308 | 328.24 | (2E,5S,8aS)-Octahydro-6-hydroxy-5,8a-dimethyl-2-{[(R)-4- | 2.68 | 328.24 |
| | | (prop-1-en-2-yl)cyclohex-1-enyl]-methylen}naphthalen-1(2H)- | | |
| | | one | | |
| 309 | 288.17 | (E,5S,8aS)-Octahydro-6-hydroxy-5,8a-dimethyl-2-[(5- | 2.74 | 288.17 |
| | | methylfuran-2-yl)methylen]naphthalen-1(2H)-one | | |
| 310 | 447.35 | (E,1S,8aS)-7-[2-(2-Cyclohexylethinyl)benzyliden]-6- | 3.10 | 447.36 |
| | | (butylamino)-decahydro-5,8a-dimethylnaphthalen-1-ol | | |
| 311 | 561.49 | (E,4aS)-6-[2-(Hexadec-1-inyl)benzyliden]-5-(butylamino)- | 3.36 | 561.52 |
| | | decahydro-1,4a-dimethylnaphthalen-2-ol | | |
| 312 | 435.30 | (1S,7E,8aS)-7-{2-[2-(Trimethylsilyl)ethinyl]benzyliden}-6- | 2.89 | 435.29 |
| | | (butylamino)-1,2,3,4,6,7,8,8a-octahydro-5,8a- | | |
| | | dimethylnaphthalen-1-ol | | |
| 313 | 459.29 | (E,1S,8aS)-7-{2-[2-(3-Fluorphenyl)ethinyl]benzyliden}-6- | 3.07 | 459.28 |
| | | (butylamino)-decahydro-5,8a-dimethylnaphthalen-1-ol | | |
| 314 | 393.27 | (1S,7E,8aS)-7-[2-(3-Methoxyprap-1-inyl)benzyliden]-6-(butyl- | 2.82 | 393.28 |
| | | amino)-1,2 3,4,6,7,8,8a-octahydro-8a-methylnaphthalen-1-ol | | |
| 315 | 435.30 | (1S,7E,8aS)-7-{2-[2-(Trimethylsilyl)ethinyl]-benzyliden}-6- | 2.91 | 435.30 |
| | | (butylamino)-1,2,3,4,6,7,8,8a-octahydro-5,8a- | | |
| | | dimethylnaphthalen-1-ol | | |
| 316 | 431.32 | (1S,7E,8aS)-7-[2-(2-Cyclohexylethinyl)benzyliden]-6-(butyl- | 2.84 | 431.32 |
| | | amino)-1,2,3,4,6,7,8,8a-octahydro-8a-methylnaphthalen-1-ol | | |
| 317 | 459.29 | (E,4aS)-6-{2-[2-(3-Fluorphenyl)ethinyl]benzyliden}-5- | 2.96 | 459.28 |
| | | (butylamino)-decahydro-1,4a-dimethylnaphthalen-2-ol | | |
| 318 | 445.33 | (1S,7E,8aS)-7-[2-(2-Cyclohexylethinyl)benzyliden]-6- | 2.74 | 445.33 |
| | | (butylamino)-1,2,3,4,6,7,8,8a-octahydro-5,8a-dimethyl- | | |
| | | naphthalen-1-ol | | |
| 319 | 409.31 | (E,1S,8aS)-7-[2-(3-Methoxyprop-1-inyl)benzyliden]-6- | 2.83 | 409.30 |
| | | (butylamino)-decahydro-5,8a-dimethylnaphthalen-1-ol | | |
| 320 | 422.33 | (E,4aS)-6-{2-[3-(Dimethylamino)prop-1-inyl]benzyliden}-5- | 2.96 | 422.33 |
| | | (butylamino)-decahydro-1,4a-dimethylnaphthalen-2-ol | | |
| 321 | 445.33 | (1S,7E,8aS)-7-[2-(Hex-1-inyl)-benzyliden]-6-(cyclohexyl- | 2.85 | 445.32 |
| | | amino)-1,2,3,4,6,7,8,8a-octahydro-5,8a-dimethylnaphthalen- | | |
| | | 1-ol | | |
| 322 | 446.33 | (1S,7E,8aS)-7-{2-[3-(Dimethylamino)prop-1-inyl]benzyliden}- | 2.97 | 446.33 |
| | | 6-(cyclohexylamino)-1,2,3,4,6,7,8,8a-octahydro-5,8a- | | |
| | | dimethylnaphthalen-1-ol | | |
| 323 | 475.38 | (E,4aS)-6-[2-(Oct-1-inyl)benzyliden]-5-(cyclohexylamino)- | 3.14 | 475.38 |
| | | decahydro-1,4a-dimethylnaphthalen-2-ol | | |
| 324 | 522.36 | (1S,7E,8aS)-7-{2-[3-(Dimethylamino)prop-1-inyl]benzyliden}- | 3.30 | 522.37 |
| | | 8a-benzyl-6-(cyclohexylamino)-1,2,3,4,6,7,8,8a-octahydro-5- | | |
| | | methylnaphthalen-1-ol | | |
| 325 | 386.17 | (3E,4aS,5S)-3-{2-[2-(3-Fluorphenyl)ethinyl]benzyliden}-4,4a, | 2.94 | 386.18 |
| | | 5,6,7,8-hexahydro-5-hydroxy-4a-methylnaphthalen-2(3H)-one | | |
| 326 | 528.40 | (3E,4R,4aS,5S)-3-[2-(Hexadec-1-inyl)benzyliden]-4a-allyl-4, | 3.27 | 528.39 |
| | | 4a,5,6,7,8-hexahydro-5-hydroxy-4-methylnaphthalen-2(3H)on | | |
| 327 | 352.20 | (E,4aS,5S)-3-[2-(3-Methoxyprop-1-inyl)benzyliden]-octahydro- | 2.81 | 352.19 |
| | | 5-hydroxy-1,4a-dimethylnaphthalen-2(1H)-one | | |
| 328 | 365.24 | (E,8aS)-2-{2-[3-(Dimethylamino)prop-1-inyl]benzyliden}- | 2.87 | 365.24 |
| | | octahydro-6-hydroxy-5,8a-dimethylnaphthalen-1(2H)-one | | |
| 329 | 400.18 | (3E,4aS,5S)-3-{2-[2-(3-Fluorphenyl)ethinyl]benzyliden}-4,4a, | 2.84 | 400.18 |
| | | 5,6,7,8-hexahydro-5-hydroxy-1,4a-dimethylnaphthalen-2(3H)- | | |
| | | one | | |
| 330 | 350.19 | (3E,4R,4aS,5S)-3-[2-(3-Methoxyprop-1-inyl)benzyliden]- | 2.91 | 350.20 |
| | | 4,4a,5,6,7,8-hexahydro-5-hydroxy-4,4a-dimethylnaphthalen- | | |
| | | 2(3H)-one | | |
| 331 | 402.20 | (E,4aS,5S)-3-{2-[2-(3-Fluorphenyl)ethinyl]benzyliden}- | 2.96 | 402.19 |
| | | octahydro-5-hydroxy-1,4a-dimethylnaphthalen-2(1H)-one | | |
| 332 | 488.37 | (3E,4aS,5S)-3-[2-(Hexadec-1-inyl)benzyliden]-4,4a,5,6,7,8- | 3.18 | 488.37 |
| | | hexahydro-5-hydroxy-4a-methylnaphthalen-2(3H)-one | | |
| 333 | 426.20 | (3E,4R,4aS,5S)-3-{2-[2-(3-Fluorphenyl)ethinyl]benzyliden}-4a- | 3.21 | 426.21 |
| | | allyl-4,4a,5,6,7,8-hexahydro-5-hydroxy-4-methylnaphthalen- | | |
| | | 2(3H)-one | | |
| 334 | 336.17 | (3E,4aS,5S)-3-[2-(3-Methoxyprop-1-inyl)benzyliden]- | 2.86 | 336.16 |
| | | 4,4a,5,6,7,8-hexahydro-5-hydroxy-4a-methylnaphthalen- | | |
| | | 2(3H)-one | | |
| 335 | 400.18 | (3E,4R,4aS,5S)-3-{2-[2-(3-Fluorphenyl)ethinyl]benzyliden}- | 2.82 | 400.18 |
| | | 4,4a,5,6,7,8-hexahydro-5-hydroxy-4,4a-dimethylnaphthalen- | | |
| | | 2(3H)-one | | |
| 336 | 364.24 | (E,8aS)-2-[2-(Hex-1-inyl)benzyliden]-octahydro-6-hydroxy- | 2.79 | 364.25 |
| | | 5,8a-dimethylnaphthalen-1(2H)-one | | |
| 337 | 350.19 | (3E,4aS,5S)-3-[2-(3-Methoxyprop-1-inyl)benzyliden]- | 2.83 | 350.20 |
| | | 4,4a,5,6,7,8-hexahydro-5-hydroxy-1,4a-dimethylnaphthalen- | | |
| | | 2(3H)-one | | |
| 338 | 426.22 | (3E,4aS,5S)-3-[2-(3-Methoxyprop-1-inyl)benzyliden]-4a- | 3.26 | 426.22 |
| | | benzyl-4,4a,5,6,7,8-hexahydro-5-hydroxy-1- | | |
| | | methylnaphthalen-2(3H)-one | | |
| 339 | 469.33 | (1S,6E,7E,8S,8aS)-7-[3-(2-Cyclohexylethinyl)benzyliden]-8a- | 3.09 | 469.34 |
| | | allyl-6-(butylimino)-1,2,3,4,6,7,8,8a-octahydro-8- | | |
| | | methylnaphthalen-1-ol | | |
| 340 | 409.30 | (E,4aS)-6-[3-(3-Methoxyprop-1-inyl)benzyliden]-5- | 2.93 | 409.30 |
| | | (butylamino)-decahydro-1,4a-dimethylnaphthalen-2-ol | | |
| 341 | 423.31 | (E,4aS)-6-[3-(3-Hydroxy-3-methylbut-1-inyl)benzyliden]-5- | 3.11 | 423.32 |
| | | (butylamino)-decahydro-1,4a-dimethylnaphthalen-2-ol | | |
| 342 | 429.30 | (1S,7E,8aS)-7-[3-(2-Cyclohexylethinyl)benzyliden]-6-(butyl- | 2.84 | 429.31 |
| | | amino)-1,2,3,4,6,7,8,8a-octahydro-8a-methylnaphthalen-1-ol | | |
| 343 | 459.29 | (E,4aS)-6-{3-[2-(2-Fluorphenyl)ethinyl]benzyliden}-5- | 2.89 | 459.30 |
| | | (butylamino)-decahydro-1,4a-dimethylnaphthalen-2-ol | | |
| 344 | 421.33 | (E,4aS)-6-[3-(Hex-1-inyl)benzyliden]-5-(butylamino)-deca- | 2.77 | 421.33 |
| | | hydro-1,4a-dimethylnaphthalen-2-ol | | |
| 345 | 409.30 | (E,1S,8aS)-7-[3-(3-Methoxyprop-1-inyl)benzyliden]-6- | 2.83 | 409.29 |
| | | (butylamino)-decahydro-5,8a-dimethylnaphthalen-1-ol | | |
| 346 | 435.31 | (E,1S,8aS)-7-[3-(3-Methoxyprop-1-inyl)benzyliden]-6- | 2.87 | 435.31 |
| | | (cyclohexylamino)-decahydro-5,8a-dimethylnaphthalen-1-ol | | |
| 347 | 525.34 | (E,1S,8S,8aR)-7-{3-[2-(3-Fluorphenyl)ethinyl]benzyliden}-8a- | 3.08 | 525.34 |
| | | allyl-6-(cyclohexylamino)-decahydro-5,8-dimethylnaphthalen- | | |
| | | 1-ol | | |
| 348 | 386.17 | (3E,4aS,5S)-3-{3-[2-(3-Fluorphenyl)ethinyl]benzyliden}-4,4a,5, | 2.72 | 386.17 |
| | | 6,7,8-hexahydro-5-hydroxy-4a-methylnaphthalen-2(3H)-one | | |
| 349 | 392.24 | (E,4R,4aR,5S)-3-[3-(3-Methoxyprop-1-inyl)benzyliden]-4a- | 3.06 | 392.23 |
| | | allyl-octahydro-5-hydroxy-1,4-dimethylnaphthalen-2(1H)-one | | |
| 350 | 400.18 | (3E,4aS,5S)-3-{3-[2-(3-Fluorphenyl)ethinyl]benzyliden}- | 2.92 | 400.19 |
| | | 4,4a,5,6,7,8-hexahydro-5-hydroxy-1,4a-dimethylnaphthalen- | | |
| | | 2(3H)-one | | |
| 351 | 352.20 | (E,8aS)-2-[3-(3-Methoxyprop-1-inyl)benzyliden]-octahydro-6- | 2.86 | 352.19 |
| | | hydroxy-5,8a-dimethylnaphthalen-1(2H)-one | | |
| 352 | 402.20 | (E,4aS,5S)-3-{3-[2-(3-Fluorphenyl)ethinyl]benzyliden}- | 2.85 | 402.21 |
| | | octahydro-5-hydroxy-1,4a-dimethylnaphthalen-2(1H)-one | | |
| 353 | 442.23 | (E,4R,4aR,5S)-3-{3-[2-(3-Fluorphenyl)ethinyl]benzyliden}-4a- | 3.04 | 442.23 |
| | | allyl-octahydro-5-hydroxy-1,4-dimethylnaphthalen-2(1H)-one | | |
| 354 | 336.17 | (3E,4aS,5S)-3-[3-(3-Methoxyprop-1-inyl)benzyliden]-4,4a,5,6, | 2.78 | 336.17 |
| | | 7,8-hexahydro-5-hydroxy-4a-methylnaphthalen-2(3H)-one | | |
| 355 | 502.38 | (3E,4aS,5S)-3-[3-(Hexadec-1-inyl)benzyliden]-4,4a,5,6,7,8- | 3.25 | 502.38 |
| | | hexahydro-5-hydroxy-1,4a-dimethylnaphthalen-2(3H)-one | | |
| 356 | 400.18 | (3E,4R,4aS,5S)-3-{3-[2-(3-Fluorphenyl)ethinyl]benzyliden}- | 2.85 | 400.19 |
| | | 4,4a,5,6,7,8-hexahydro-5-hydroxy-4,4a-dimethylnaphthalen- | | |
| | | 2(3H)-one | | |
| 357 | 350.19 | (3E,4aS,5S)-3-[3-(3-Methoxyprop-1-inyl)benzyliden]-4,4a,5,6, | 2.84 | 350.19 |
| | | 7,8-hexahydro-5-hydroxy-1,4a-dimethylnaphthalen-2(3H)-on | | |
| 358 | 426.22 | (3E,4aS,5S)-3-[3-(3-Methoxyprop-1-inyl)benzyliden]-4a- | 3.16 | 426.22 |
| | | benzyl-4,4a,5,6,7,8-hexahydro-5-hydroxy-1- | | |
| | | methylnaphthalen-2(3H)-one | | |
| 359 | 409.30 | (E,1S,8aS)-7-[3-(3-Methoxyprop-1-inyl)benzyliden]-6- | 2.91 | 409.31 |
| | | (butylamino)-decahydro-5,8a-dimethylnaphthalen-1-ol | | |
| 360 | 447.35 | (E,1S,8aS)-7-[3-(2-Cyclohexylethinyl)benzyliden]-6- | 2.86 | 447.34 |
| | | (butylamino)-decahydro-5,8a-dimethylnaphthalen-1-ol | | |
| 361 | 409.30 | (E,4aS)-6-[3-(3-Methoxyprop-1-inyl)benzyliden]-5- | 2.94 | 409.29 |
| | | (butylamino)-decahydro-1,4a-dimethylnaphthalen-2-ol | | |
| 362 | 499.30 | (E,1S,8S,8aR)-7-{3-[2-(3-Fluorphenyl)ethinyl]benzyliden}-8a- | 3.20 | 499.30 |
| | | allyl-6-(butylamino)-decahydro-5,8-dimethylnaphthalen-1-ol | | |
| 363 | 423.31 | (E,4aS)-6-[3-(3-Hydroxy-3-methylbut-1-inyl)benzyliden]-5- | 3.03 | 423.29 |
| | | (butylamino)-decahydro-1,4a-dimethylnaphthalen-2-ol | | |
| 364 | 431.32 | (1S,7E,8aS)-7-[3-(2-Cyclohexylethinyl)benzyliden]-6-(butyl- | 2.92 | 431.33 |
| | | amino)-1,2,3,4,6,7,8,8a-octahydro-8a-methylnaphthalen-1-ol | | |
| 365 | 459.29 | (E,4aS)-6-{3-[2-(3-Fluorphenyl)ethinyl]benzyliden}-5- | 3.04 | 459.28 |
| | | (butylamino)-decahydro-1,4a-dimethylnaphthalen-2-ol | | |
| 366 | 407.28 | (1S,7E,8aS)-7-[3-(3-Hydroxy-3-methylbut-1-inyl)benzyliden]- | 3.22 | 407.27 |
| | | 6-(butylamino)-1,2,3,4,6,7,8,8a-octahydro-8a- | | |
| | | methylnaphthalen-1-ol | | |
| 367 | 449.33 | (E,1S,8S,8aR)-7-[3-(3-Methoxyprop-1-inyl)benzyliden]-8a- | 3.18 | 449.33 |
| | | allyl-6-(butylamino)-decahydro-5,8-dimethylnaphthalen-1-ol | | |
| 368 | 422.33 | (E,4aS)-6-{3-[3-(Dimethylamino)prop-1-inyl]benzyliden}-5- | 2.97 | 422.32 |
| | | (butylamino)-decahydro-1,4a-dimethylnaphthalen-2-ol | | |
| 369 | 485.31 | (E,4aS)-6-{3-[2-(3-Fluorphenyl)ethinyl]benzyliden}-5- | 3.12 | 485.30 |
| | | (cyclohexylamino)-decahydro-1,4a-dimethylnaphthalen-2-ol | | |
| 370 | 559.33 | (1S,7E,8aS)-7-{3-[2-(3-Fluorphenyl)ethinyl]benzyliden}-8a- | 3.26 | 559.33 |
| | | benzyl-6-(cyclohexylamino)-1,2,3,4,6,7,8,8a-octahydro-5- | | |
| | | methylnaphthalen-1-ol | | |
| 371 | 435.31 | (E,4aS)-6-[3-(3-Methoxyprop-1-inyl)benzyliden]-5-(cyclohexyl- | 2.84 | 435.32 |
| | | amino)-decahydro-1,4a-dimethylnaphthalen-2-ol | | |
| 372 | 473.37 | (E,4aS)-6-[3-(2-Cyclohexylethinyl)benzyliden]-5-(cyclohexyl- | 2.81 | 473.38 |
| | | amino)-decahydro-1,4a-dimethylnaphthalen-2-ol | | |
| 373 | 431.32 | (E,4aS)-6-[3-(3-Methylbut-3-en-1-inyl)benzyliden]-5- | 2.86 | 431.32 |
| | | (cyclohexylamino)-decahydro-1,4a-dimethylnaphthalen-2-ol | | |
| 374 | 386.17 | (3E,4aS,5S)-3-{4-[2-(3-Fluorphenyl)ethinyl]benzyliden}- | 2.91 | 386.17 |
| | | 4,4a,5,6,7,8-hexahydro-5-hydroxy-4a-methylnaphthalen- | | |
| | | 2(3H)-one | | |
| 375 | 352.20 | (E,4aS,5S)-3-[4-(3-Methoxyprop-1-inyl)benzyliden]-octahydro- | 2.96 | 352.20 |
| | | 5-hydroxy-1,4a-dimethylnaphthalen-2(1H)-one | | |
| 376 | 363.22 | (3E,4R,4aS,5S)-3-{4-[3-(Dimethylamino)prop-1- | 2.87 | 363.22 |
| | | inyl]benzyliden}-4,4a,5,6,7,8-hexahydro-5-hydroxy-4,4a- | | |
| | | dimethylnaphthalen-2(3H)-one | | |
| 377 | 376.20 | (3E,4R,4aS,5S)-3-[4-(3-Methoxyprop-1-inyl)benzyliden]-4a- | 3.08 | 376.19 |
| | | allyl-4,4a,5,6,7,8-hexahydro-5-hydroxy-4-methylnaphthalen- | | |
| | | 2(3H)-one | | |
| 378 | 400.18 | (3E,4aS,5S)-3-{4-[2-(3-Fluorphenyl)ethinyl]benzyliden}- | 2.96 | 400.18 |
| | | 4,4a,5,6,7,8-hexahydro-5-hydroxy-1,4a-dimethylnaphthalen- | | |
| | | 2(3H)-one | | |
| 379 | 476.22 | (3E,4aS,5S)-3-{4-[2-(3-Fluorphenyl)ethinyl]benzyliden}-4a- | 3.16 | 476.21 |
| | | benzyl-4,4a,5,6,7,8-hexahydro-5-hydroxy-1- | | |
| | | methylnaphthalen-2(3H)-one | | |
| 380 | 414.26 | (3E,4R,4aS,5S)-3-[4-(2-Cyclohexylethinyl)benzyliden]-4a- | 3.07 | 414.26 |
| | | allyl-4,4a,5,6,7,8-hexahydro-5-hydroxy-4-methylnaphthalen- | | |
| | | 2(3H)-one | | |
| 381 | 402.20 | (E,4aS,5S)-3-{4-[2-(3-Fluorphenyl)ethinyl]benzyliden}- | 2.87 | 402.20 |
| | | octahydro-5-hydroxy-1,4a-dimethylnaphthalen-2(1H)-one | | |
| 382 | 402.26 | (3E,4R,4aS,5S)-3-[4-(3-Cyclohexylprop-1-inyl)benzyliden]- | 2.81 | 402.25 |
| | | 4,4a,5,6,7,8-hexahydro-5-hydroxy-4,4a-dimethylnaphthalen- | | |
| | | 2(3H)-one | | |
| 383 | 336.17 | (3E,4aS,5S)-3-[4-(3-Methoxyprop-1-inyl)benzyliden]-4,4a,5,6, | 2.89 | 336.17 |
| | | 7,8-hexahydro-5-hydroxy-4a-methylnaphthalen-2(3H)-one | | |
| 384 | 402.20 | (E,8aS)-2-{4-[2-(3-Fluorphenyl)-ethinyl]benzyliden}-octahydro- | 2.76 | 402.21 |
| | | 6-hydroxy-5,8a-dimethylnaphthalen-1(2H)-one | | |
| 385 | 350.19 | (3E,4aS,5S)-3-[4-(3-Methoxyprop-1-inyl)benzyliden]-4,4a,5,6, | 2.89 | 350.18 |
| | | 7,8-hexahydro-5-hydroxy-1,4a-dimethylnaphthalen-2(3H)-on | | |
| 386 | 426.22 | (3E,4aS,5S)-3-[4-(3-Methoxyprop-1-inyl)benzyliden]-4a- | 3.21 | 426.23 |
| | | benzyl-4,4a,5,6,7,8-hexahydro-5-hydroxy-1- | | |
| | | methylnaphthalen-2(3H)-one | | |
| 387 | 389.24 | (3E,4R,4aS,5S)-3-{4-[3-(Dimethylamino)prop-1- | 3.07 | 389.23 |
| | | inyl]benzyliden}-4a-allyl-4,4a,5,6,7,8-hexahydro-5-hydroxy-4- | | |
| | | methylnaphthalen-2(3H)-one | | |
| 388 | 251.22 | (1S,8aS)-6-(Diethylamino)-1,2,3,4,6,7,8,8a-octahydro-5,8a- | 2.72 | 251.22 |
| | | dimethylnaphthalen-1-ol | | |
| 389 | 251.22 | (1S,8S,8aS)-6-(Diethylamino)-1,2,3,4,6,7,8,8a-octahydro- | 2.74 | 251.22 |
| | | 8,8a-dimethylnaphthalen-1-ol | | |
| 390 | 237.21 | (1S,8aS)-6-(Diethylamino)-1,2,3,4,6,7,8,8a-octahydro-8a- | 2.69 | 237.20 |
| | | methylnaphthalen-1-ol | | |
| 391 | 327.26 | (1S,8aR)-8a-Benzyl-6-(diethylamino)-1,2,3,4,6,7,8,8a- | 2.85 | 327.28 |
| | | octahydro-5-methylnaphthalen-1-ol | | |
| 392 | 277.24 | (1S,8S,8aS)-8a-Allyl-6-(diethylamino)-1,2,3,4,6,7,8,8a- | 2.83 | 277.24 |
| | | octahydro-8-methylnaphthalen-1-ol | | |
| 393 | | (6E,4aS)-2-Furan-2-ylmethylene-5,8a-dimethyl-6-methylene- | | |
| | | 3,4,6,7,8,8a-hexahydro-2H-naphthalen-1-one | | |
| 394 | | 2-Furan-2-ylmethylene-8-methyl-3,4,4a,5,8,8a-hexahydro-2H- | | |
| | | naphthalen-1-one | | |
| 395 | | (4aS,5S)-5-Hydroxy-1,4a-dimethyl-3-(4-pyridin-4-ylethynyl- | | |
| | | benzylidene)-4,4a,5,6,7,8-hexahydro-3H-naphthalen-2-one | | |
| 396 | | (4aS,5S)-3-[4-(3-Dimethylamino-prop-1-ynyl)-benzylidene]-5- | | |
| | | hydroxy-1,4a-dimethyl-4,4a,5,6,7,8-hexahydro-3H- | | |
| | | naphthalen-2-one | | |
| 397 | | (4aS,5S)-5-Hydroxy-1,4a-dimethyl-1-(3-phenyl-propyl)- | | |
| | | octahydro-naphthalen-2-one | | |
| 398 | | (4aS,5S)-5-Hydroxy-1-(3-iodo-benzyl)-1,4a-dimethyl- | | |
| | | octahydro-naphthalen-2-one | | |
| 399 | | (4aS,5S)-3-(4-Cyclohexylamino-benzylidene)-5-hydroxy-1,4a- | | |
| | | dimethyl-4,4a,5,6,7,8-hexahydro-3H-naphthalen-2-one | | |
| 400 | | (4aS)-5,8a-Dimethyl-7-(5-methyl-furan-2-ylmethylene)- | | |
| | | hexahydro-naphthalen-1,6-dione | | |
| 401 | | (2E,4aS)-5-(1,4a-Dimethyl-5-oxo-4,4a,5,6,7,8-hexahydro-3H- | | |
| | | naphthalen-2-ylidene)-5H-furan-2-one | | |
| 402 | | (7′E)-7′-(4-iodo-benzylidene)-5′,8a′-dimethylhexahydro-5′-(2- | | |
| | | phenylethyl)-2′H-spiro[1,3-dioxolane-2,1′-naphthalen]-6′(5′H)- | | |
| | | one | | |
| 403 | | 2-Furan-2-ylmethylene-3,4,4a,5,8,8a-hexahydro-2H- | | |
| | | naphthalen-1-one | | |
| 404 | | (4aS)-7-Benzylidene-5,8a-dimethyl-hexahydro-naphthalen- | | |
| | | 1,6-dione | | |
| 405 | | (4aS,5S)-5-Hydroxy-1,4a-dimethyl-1-(2-nitro-benzyl)- | | |
| | | octahydro-naphthalen-2-one | | |
| 406 | | (4aS,5S)-5-Hydroxy-1-isobutyl-1,4a-dimethyl-1-octahydro- | | |
| | | naphthalen-2-one | | |
| 407 | | 8-Methyl-3,4,4a,5,8,8a-hexahydro-2H-naphthalen-1-one | | |
| 408 | | (3E,4aS)-5,8a-Dimethyl-7-(2,4,6-trimethoxy-benzylidene)- | | |
| | | 3,4,8,8a-tetrahydro-2H,7H-naphthalen-1,6-dione | | |
| 409 | | (3E,3′E,4aS,4a′S)-1,3-bis[5,8a-dimethyl-3,4,8,8a-tetrahydro- | | |
| | | 2H,7H-naphthalen-1,6-dione]-benzene | | |
| 410 | | (4aS)-5-Butyliminomethyl-8a-methyl-3,4,8,8a-tetrahydro- | | |
| | | 2H,7H-naphthalen-1,6-dione | | |
| 411 | | (2E,4aS,5S)-6-Ethylidene-5,8a-dimethyl-1,2,3,4,6,7,8,8a- | | |
| | | octahydro-naphthalen-1-ol | | |
| 412 | | (4aS,5S)-5-Hydroxy-3-[1-(4-iodo-phenyl)-propyl]-1,4a- | | |
| | | dimethyl-4,4a,5,6,7,8-hexahydro-3H-naphthalen-2-one | | |
| 413 | | (4aS,5S)-5-Hydroxy-1,4a-dimethyl-4,4a,5,6,7,8-hexahydro- | | |
| | | 3H-naphthalen-2-one | | |
| 414 | | (4aS,5S)-1-Butyl-5-hydroxy-1,4a-dimethyl-1-octahydro- | | |
| | | naphthalen-2-one | | |
| 415 | | (5E)-5-(5′,8a′-dimethyl-3′,4′,8′,8a′-tetrahydro-2′H-spiro[1,3- | | |
| | | dioxolane-2,1′-naphthalen]-6′(7′H)-ylidene)furan-2(5H)-one | | |
| 416 | | (7′E)-5′-(3,5-Dibromobenzyl)-7′-(4-iodo-benzylidene)-5′,8a′- | | |
| | | dimethylhexahydro-2′H-spiro[1,3-dioxolane-2,1′-naphthalen]- | | |
| | | 6′(5′H)-one | | |
| 417 | | 5-Hydroxy-7-isopropyl-1-methyl-4,4a,5,6,7,8,-hexahydro-3H- | | |
| | | naphthalen-2-one | | |
| 418 | | (4aS,5S)-5-Hydroxy-1,4a-dimethyl-1-octahydro-naphthalen-2- | | |
| | | one | | |

The present invention also comprises pharmaceutically acceptable salts of the compounds according to the general formula (I), all stereoisomeric forms of the compounds according to the general formula (I) as well as solvates, especially hydrates or prodrugs thereof. A prodrug is commonly described as an inactive or protected derivative of an active ingredient or a drug, which is converted to the active ingredient or drug in the body.

In case, the inventive compounds bear basic and/or acidic substituents, they may form salts with organic or inorganic acids or bases. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, d-o-tolyltartaric acid, tartronic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner. Examples for suitable inorganic or organic bases are, for example, NaOH, KOH, NH₄OH, tetraalkylammonium hydroxide, lysine or arginine and the like. Salts may be prepared in a conventional manner using methods well known in the art, for example by treatment of a solution of the compound of the general formula (I) with a solution of an acid, selected out of the group mentioned above.

Some of the compounds of the present invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain compounds of the general formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses. Where a compound according to the general formula (I) contains an alkene moiety, the alkene can be presented as a cis or trans isomer or a mixture thereof. When an isomeric form of a compound of the invention is provided substantially free of other isomers, it will preferably contain less than 5% w/w, more preferably less than 2% w/w and especially less than 1% w/w of the other isomers.

Another aspect of the present invention relates to the use of the inventive decaline derivatives as drugs, i.d. as pharmaceutically active agents applicable in medicine. Furthermore, it was found that the decaline derivatives of the present invention are inhibitors of kinases and phosphatases, especially of 11βHSD1 (11β-hydroxysteroid dehydrogenase type 1).

The 11βHSD2 isoenzyme catalyzes exclusively the oxidation of cortisol, and inhibition of 11βHSD2 causes sodium retention resulting in hypertension. Therefore isoenzyme-specificity is a major prerequisite for the clinical use of 11βHSD1 inhibitors. 11βHSD1 inhibitors can be applied in the treatment of several diseases, such as the metabolic syndrome, diabetes type 2, obesity and cognitive dysfunction. Moreover, 11betaHSD1 inhibition slows down plaque progression in the context of atherosclerosis (Hermanowski-Vosatka et al., The Journal of Experimental Medicine, 2005, 202, 4, 517-527). 11betaHSD1 inhibition also offers a novel therapeutic approach to improve healing of ischemic or injured tissue (Small et al., Proc. Natl. Acad. Sci. U.S.A. 2005, 102, 34, 12165-12170). Within the present decaline derivatives also inhibitors for a panel of phosphatases including Cdc25A phosphatase, VHR, PTP1B, PP1 and MtPTPA as well as for acetylcholinesterase could be identified.

Cdc25A is a dual-specificity phosphatase that regulates progression of cell division at the G1→S checkpoint by dephosphorylating Cdk2/cyclin complexes. Cdc25A is considered a viable target in the development of new anti-tumor drugs. PTP1B (protein tyrosine phosphatase 1B) is thought to function as a negative regulator of insulin and leptin signal transduction. Increased incidence of type 2 diabetes mellitus and obesity has elevated the medical need for new agents to treat these disease states. Resistance to the hormones insulin and leptin are hallmarks of both type 2 diabetes and obesity. Drugs that can ameliorate this resistance should be effective in treating type 2 diabetes and possibly obesity. PTP1B can therefore be regarded as a novel target for type 2 diabetes.

VHR affects the MAPK pathway by dephosphorylating ERK (extracellular regulated kinase) 1 and ERK2 as well as JNK (c-Jun N-terminal kinase), which leads in all three cases to their inactivation. VHR is related to cellular proliferation and inflammation processes.

PP1 is a Ser/Thr-phosphatase that plays an important role in cellular proliferation and differentiation, since it can dephosphorylate tumor suppressor protein Retinoblastoma (pRb) so that transcription cannot take place. PP1 is considered an interesting target in the treatment of various cancers.

MtPTPA (a protein tyrosine phosphatase of Mycobacterium tuberculosis) seems to disturb phosphotyrosine-mediated signal transduction in the immune system of the host organism and may therefore represent a viable target in the treatment of tuberculosis.

Acetylcholinesterase (AChE) hydrolyzes the neurotransmitter acetylcholine and thereby terminates impulse transmission at cholinergic synapses. AChE inhibitors are used in the treatment of various disorders such as myasthenia gravis, glaucoma and Alzheimer's disease.

Table II (cf. below) shows the half-maximal inhibition concentration (IC₅₀) values of representative compounds according to general formula (I). Table II shows inhibition rates with IC₅₀ between 10 and 50 μM and below 10 μM. The results exhibited table II prove that the compounds of the present invention are potent pharmaceutically active agents against various diseases that can be treated and/or prohibited by inhibition of the targets \textcircled{2}-\textcircled{9}.

TABLE II
Inhibitory effect of the compounds of the present invention on different
targets (a = 50 μM > IC₅₀ > 10 μM & aa = IC₅₀ < 10 μM)

| \textcircled{1} | \textcircled{2} | \textcircled{3} | \textcircled{4} | \textcircled{5} | \textcircled{6} | \textcircled{7} | \textcircled{8} | \textcircled{9} |
|---|---|---|---|---|---|---|---|---|
| 1 | a | | | | | aa | | |
| 3, 16, 27, 30, 33, 39, 43, 58, 79, | a | a | | | | | | |
| 395, 406 | | | | | | | | |
| 7, 18, 20, 68, 89, 107, 115, 126, | a | | | | | | | |
| 136, 138, 143, 146, 153, 190, | | | | | | | | |
| 198, 202, 203, 205, 215, 223, | | | | | | | | |
| 225, 241, 242, 266, 267, 286, | | | | | | | | |
| 347, 362, 400, 402, 404, 405, | | | | | | | | |
| 408, 409, 410 | | | | | | | | |
| 8, 9 | aa | a | a | | | | | |
| 60, 62, 393, 398 | a | a | a | | | | | |
| 394 | a | aa | a | | | | | |
| 19 | aa | | | | | | | |
| 24 | a | a | a | | a | aa | | |
| 25 | a | | | a | a | | | |
| 26 | a | a | | a | | aa | a | |
| 32 | aa | a | a | a | aa | aa | aa | aa |
| 38, 46, 54 | a | a | aa | a | | | | |
| 40, 44 | a | a | a | a | | | | |
| 56, 158, 396, 412, 413 | a | a | | | a | | | |
| 45 | a | a | | aa | | | | |
| 47 | a | a | a | aa | | | | |
| 49 | a | | aa | aa | a | | | |
| 50, 55 | a | a | aa | | a | | | |
| 51 | a | a | aa | | | | | |
| 397 | a | | a | a | | | | |
| 333, 401 | a | | a | | | | | |
| 137, 138 | a | | aa | | | | | |
| 125, 127, 128, 130, 131, 133, | a | | | | a | | | |
| 134, 141 | | | | | | | | |
| 4 | | a | a | a | | aa | | |
| 12 | | a | | a | | aa | | |
| 17 | | a | | a | | | | |
| 29 | | aa | a | | | aa | aa | aa |
| 31 | | a | | | | aa | a | aa |
| 34, 37, 41, 59, 61, 63, 66, 90, | | a | | | | | | |
| 282, 417 | | | | | | | | |
| 53 | | a | a | a | | | | |
| 64, 414 | | a | a | | | | | |
| 83, 416 | | a | | | | aa | | |
| 42, 91 | | | aa | | | | | |
| 48, 67, 71, 72, 86, 323, 325, 326, | | | a | | | | | |
| 332, 334, 335, 346, 349, 350, | | | | | | | | |
| 353, 415 | | | | | | | | |
| 411 | | | | a | | | | |
| 388 | | | | | a | | | |
| 10, 11, 14, 22, 23, 73, 83, 85, 97, | | | | | | aa | | |
| 98, 399, 407 | | | | | | | | |
| 15 | | | | | | aa | a | |
| 65 | | | | | | a | | |
| 403 | | | | | | aa | aa | |
| 167 | | | | | | | | aa |

\textcircled{1} Compound Number
\textcircled{2} Target VHR
\textcircled{3} Target PTP1B
\textcircled{4} Target Cdc25A
\textcircled{5} Target PP1
\textcircled{6} Target MPTPA
\textcircled{7} Target 11beta-HSD1
\textcircled{8} Target 11beta-HSD2
\textcircled{9} Target AChE

Thus, the decaline compounds of the present invention can be used for prophylaxis and treatment of diabetes mellitus type I, diabetes mellitus type II, tuberculosis, proliferative diseases, cancer, infectious diseases, neurodegenerative diseases, obesity, cognitive dysfunctions, metabolic syndromes or for the preparation of a pharmaceutical formulation for prophylaxis and treatment of diabetes mellitus type I, diabetes mellitus type II, tuberculosis, proliferative diseases, cancer, infectious diseases, neurodegenerative diseases, obesity, cognitive dysfunctions, metabolic syndromes.

Examples for neurodegenerative diseases are Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA). Alzheimer disease and Parkinson disease are two mayor indications for the decaline-derived compounds of the present invention.

Furthermore, the compounds of the present invention are useful for the treatment of various cancer types such as adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

Most active are the inventive compounds on lung cancer, lymph node cancer (Hodgkin's/Non-Hodgkin's), head and neck tumors (tumors of the ear, nose and throat area), breast cancer, ovarian cancer, gastric cancer, gastrointestinal tumors, intestinal tumors.

Furthermore, the compounds of the general formulas disclosed herein were identified as angiogenesis inhibitors which are useful for cancer treatment by blocking the formation of vessels into the tumor or tumorous tissue.

Also infectious diseases including opportunistic diseases can be treated by the decaline-derived compounds of the present invention. Such infectious diseases comprise for instance AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever, tuberculosis, leprosy, mycobacteria-induced meningitis.

Especially tuberculosis is one important indication for the present compounds.

Further indications are cognitive disorders. The term “cognitive disorder” shall refer to anxiety disorders, delirium, dementia, amnestic disorders, dissociative disorders, eating disorders, mood disorders, schizophrenia, psychotic disorders, sexual and gender identity disorders, sleep disorders, somatoform disorders, acute stress disorder, obsessive-compulsive disorder, panic disorder, posttraumatic stress disorder, specific phobia, social phobia, substance withdrawal delirium, Alzheimer's disease, Creutzfeldt-Jakob disease, head trauma, Huntington's disease, HIV disease, Parkinson's disease, Pick's disease, learning disorders, motor skills disorders, developmental coordination disorder, communication disorders, phonological disorder, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, pica, rumination disorder, tic disorders, chronic motor or vocal tic disorder, Tourette's disorder, elimination disorders, encopresis, enuresis, selective mutism, separation anxiety disorder, dissociative amnesia, depersonalization disorder, dissociative fugue, dissociative identity disorder, anorexia nervosa, bulimia nervosa, bipolar disorders, schizophreniform disorder, schizoaffective disorder, delusional disorder, psychotic disorder, shared psychotic disorder, delusions, hallucinations, substance-induced psychotic disorder, orgasmic disorders, sexual pain disorders, dyspareunia, vaginismus, sexual dysfunction, paraphilias, dyssomnias, breathing-related sleep disorder, circadian rhythm sleep disorder, hypersomnia, insomnia, narcolepsy, dyssomnia, parasomnias, nightmare disorder, sleep terror disorder, sleepwalking disorder, parasomnia, body dysmorphic disorder, conversion disorder, hypochondriasis, pain disorder, somatization disorder, alcohol related disorders, amphetamine related disorders, caffeine related disorders, cannabis related disorders, cocaine related disorders, hallucinogen related disorders, inhalant related disorders, nicotine related disorders, opioid related disorders, phencyclidine-related disorder, abuse, persisting amnestic disorder, intoxication, withdrawal.

It was also found that the compounds disclosed herein are useful for the treatment and prophylaxis of the Cushing's syndrome and Addison's disease two very serious, potentially lethal clinical disorders, which are related to glucocorticoid excess or deficiency (glucocorticoid dysfunction) as well as diseases which are caused or induced by disorders or dysfunction of the glucocorticoid signaling cascade. Such diseases are, for instance, glucocorticoid hypersensitivity, glucocorticoid resistance, hypercortisolism, obesity, the dysmetabolic syndrome, diabetes type 2, melancholic depression, osteoporosis, anxiety, insomnia, asthenia, depression, memory dysfunction, executive dysfunction, fatigue, hyperalgesia, poor quality sleep, insulin resistance, atrophy (sarcopenia), growth stunting, hypertension.

Another aspect of the present invention is directed to the use of at least one compound of any one of the general formulas disclosed herein and/or pharmaceutically acceptable salts thereof for prophylaxis and/or treatment of cardiovascular diseases, cardiovascular disorders and metabolic syndromes.

The metabolic syndrome is a cluster of cardiovascular risk factors, including visceral obesity, insulin resistance, dyslipidemia, obesity, hypertension, impaired glucose tolerance and hypertriglyceridemia.

Examples of cardiovascular diseases and disorders are: adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, atherosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

Another important aspect of the present invention deals with the use of the decaline-derived compounds in combination with common drugs such as anti-HIV drugs, antiproliferative drug, cytotoxic or cytostatic drug, ganciclovir, foscarnet, cidofovir, valganciclovir, fomivirsen, penciclovir or valaciclovir. In some cases, the inventive compounds are able to increase the activity of the common drugs and/or reduce their undesired side effects.

Therefore, another aspect of the present invention is directed to pharmaceutical compositions comprising at least one compound of the present invention as active ingredient, together with at least one pharmaceutically acceptable carrier, excipient and/or diluents. The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain at least one compound according to the present invention and/or a pharmaceutical acceptable salt thereof as active ingredient.

The pharmaceutical compositions according to the present invention containing at least one compound according to the present invention, and/or a pharmaceutical acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, extrudates, deposits, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like. Moreover, suitable binders, lubricants, disintegrating agents and coloring agents may also be incorporated into the tablet or capsule. Powders and tablets may contain about 5 to about 95 weight % of the decalin-derived compound and/or the respective pharmaceutically active salt as active ingredient.

Suitable binders include starch, gelatin, natural sugars, corn sweeteners, natural and synthetic gums such as acacia, sodium alginate, carboxymethylcellulose, polyethylene glycol and waxes. Among suitable lubricants there may be mentioned boric acid, sodium benzoate, sodium acetate, sodium chloride, and the like. Suitable disintegrants include starch, methylcellulose, guar gum, and the like. Sweetening and flavoring agents as well as preservatives may also be included, where appropriate. The disintegrants, diluents, lubricants, binders etc. are discussed in more detail below.

Moreover, the pharmaceutical compositions of the present invention may be formulated in sustained release form to provide the rate controlled release of any one or more of the components or active ingredients to optimise the therapeutic effect(s), e.g. antihistaminic activity and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

Liquid form preparations include solutions, suspensions, and emulsions. As an example, there may be mentioned water or water/propylene glycol solutions for parenteral injections or addition of sweeteners and opacifiers for oral solutions, suspensions, and emulsions. Liquid form preparations may also include solutions for intranasal administration. Aerosol preparations suitable for inhalation may include solutions and solids in powder form, which may be present in combination with a pharmaceutically acceptable carrier such as an inert, compressed gas, e.g. nitrogen. For preparing suppositories, a low melting fat or wax, such as a mixture of fatty acid glycerides like cocoa butter is melted first, and the active ingredient is then dispersed homogeneously therein e.g. by stirring. The molten, homogeneous mixture is then poured into conveniently sized moulds, allowed to cool, and thereby solidified,

Also included are solid form preparations which are intended to be converted, shortly before use, to liquid form preparations for either oral or parenteral administration. Such liquid forms include solutions, suspensions, and emulsions.

The compounds according to the present invention may also be delivered transdermally. The transdermal compositions may have the form of a cream, a lotion, an aerosol and/or an emulsion and may be included in a transdermal patch of the matrix or reservoir type as is known in the art for this purpose.

The term capsule as recited herein refers to a specific container or enclosure made e.g. of methyl cellulose, polyvinyl alcohols, or denatured gelatins or starch for holding or containing compositions comprising the active ingredient(s). Capsules with hard shells are typically made of blended of relatively high gel strength gelatins from bones or pork skin. The capsule itself may contain small amounts of dyes, opaquing agents, plasticisers and/or preservatives. Under tablet a compressed or moulded solid dosage form is understood which comprises the active ingredients with suitable diluents. The tablet may be prepared by compression of mixtures or granulations obtained by wet granulation, dry granulation, or by compaction well known to a person of ordinary skill in the art.

Oral gels refer to the active ingredients dispersed or solubilised in a hydrophilic semi-solid matrix. Powders for constitution refers to powder blends containing the active ingredients and suitable diluents which can be suspended e.g. in water or in juice.

Suitable diluents are substances that usually make up the major portion of the composition or dosage form. Suitable diluents include sugars such as lactose, sucrose, mannitol, and sorbitol, starches derived from wheat, corn rice, and potato, and celluloses such as microcrystalline cellulose. The amount of diluent in the composition can range from about 5 to about 95% by weight of the total composition, preferably from about 25 to about 75 weight %, and more preferably from about 30 to about 60 weight %.

The term disintegrants refers to materials added to the composition to support break apart (disintegrate) and release the pharmaceutically active ingredients of a medicament. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodium croscaramellose, alginates such as alginic acid and sodium alginate, clays such as bentonites, and effervescent mixtures. The amount of disintegrant in the composition may range from about 2 to about 20 weight % of the composition, more preferably from about 5 to about 10 weight %.

Binders are substances which bind or “glue” together powder particles and make them cohesive by forming granules, thus serving as the “adhesive” in the formulation. Binders add cohesive strength already available in the diluent or bulking agent. Suitable binders include sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate. The amount of binder in the composition may range from about 2 to about 20 weight % of the composition, preferably from about 3 to about 10 weight %, and more preferably from about 3 to about 6 weight %.

Lubricants refer to a class of substances which are added to the dosage form to enable the tablet granules etc. after being compressed to release from the mould or die by reducing friction or wear. Suitable lubricants include metallic stearates such as magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine. Lubricants are usually added at the very last step before compression, since they must be present at the surface of the granules. The amount of lubricant in the composition may range from about 0.2 to about 5 weight % of the composition, preferably from about 0.5 to about 2 weight %, and more preferably from about 0.3 to about 1.5 weight % of the composition.

Glidents are materials that prevent caking of the components of the pharmaceutical composition and improve the flow characteristics of granulate so that flow is smooth and uniform. Suitable glidents include silicon dioxide and talc. The amount of glident in the composition may range from about 0.1 to about 5 weight % of the final composition, preferably from about 0.5 to about 2 weight %.

Coloring agents are excipients that provide coloration to the composition or the dosage form. Such excipients can include food grade dyes adsorbed onto a suitable adsorbent such as clay or aluminum oxide. The amount of the coloring agent may vary from about 0.1 to about 5 weight % of the composition, preferably from about 0.1 to about 1 weight %.

### DESCRIPTION OF FIGURES

FIG. 1 shows two important general subformula for two important subgroup of compounds of the present invention having the decaline scaffold,

FIG. 2 shows representative examples of the inventive decaline-derived compounds,

FIG. 3 shows representative examples of the inventive decaline-derived compounds.

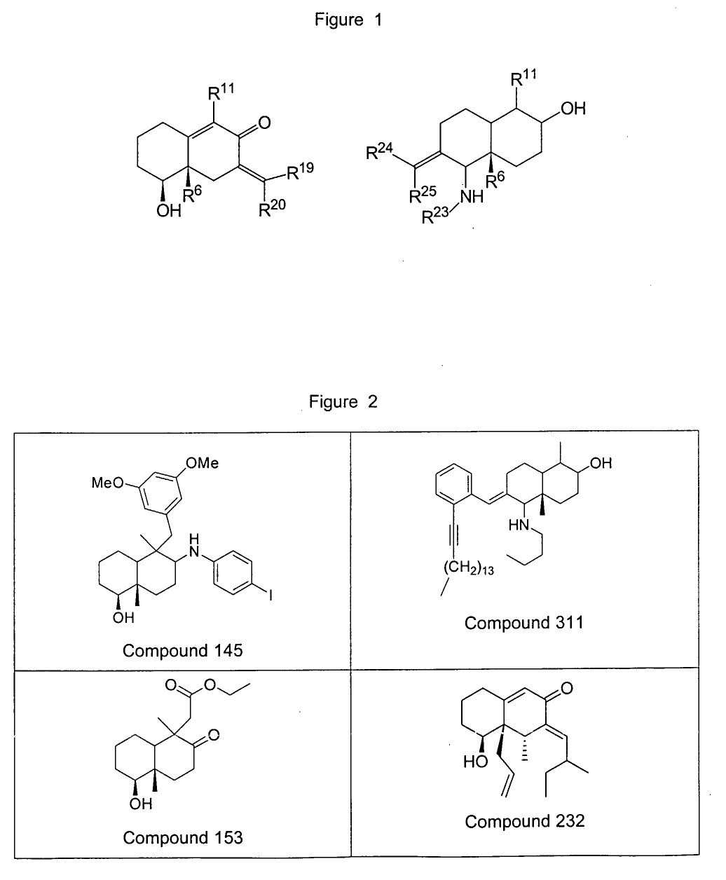

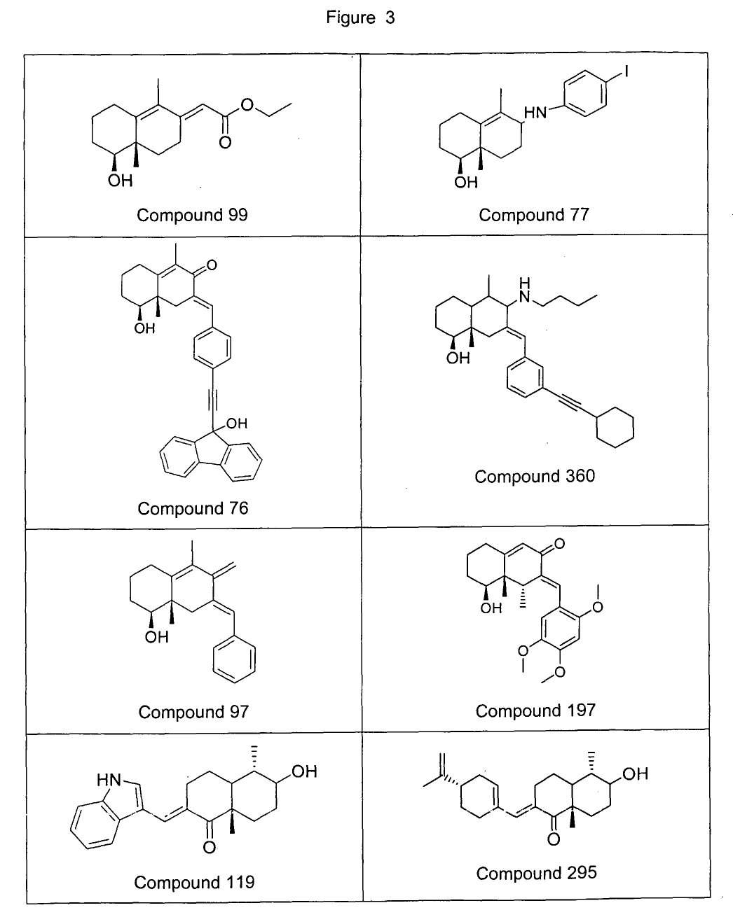

### EXPERIMENTAL PART

### Analytical HPLC-MS Method

Analytical HPLC/MS was performed on:

### 1.) For Substance 1-158:

HP Model Hewlett Packard Series 1100 coupled to a Finnigan MAT LCQ mass detector. UV spectra recording is integrated in the HP Model Hewlett Packard Series 1100. UV spectra were recorded 210 and 254 nm.

Chromatography was performed using the parameters cited below:

| | |
|---|---|
| Solvents: | Acetonitrile (Biosolve) |
| | Water (Millipore) |
| Column: | Macherey&Nagel CC 125/4 Nucleosil 120-5 C4 |
| Flow rate: | 0.5 ml/min |
| Gradient: | A: Water/0.1% TFA B: Acetonitrile/0.1% TFA |

| Time | A % | B % |
|---|---|---|
| 0.00 | 90 | 10 |
| 2.00 | 30 | 70 |
| 10.00 | 100 | 0 |

UV spectra were recorded at 210 and 254 nm with a sampling rate of 0.5 spectra/second. Mass spectra were obtained using positive and negative electrospray ionization over the range m/z 70 to 900. The scan rate was 0.5 scan (m/z 70 to 900) per second.

### 2.) For Substance 159-392:

Micromass LCT: HPLC Waters 600 coupled to a Micromass TOF-MUX-Interface. UV spectra recording is integrated in the Micromass LCT. UV spectra were recorded at 210 nm.

Chromatography was performed using the parameters cited below:

| | |
|---|---|
| Solvents: | Acetonitrile (Biosolve) |
| | Water (Millipore) |
| Column: | Phenomenex Synergi 2μ Hydro-RP Mercury 20 mm × 4 mm |
| Flow rate: | 1.0 ml/min |
| Gradient: | A: Water/0.1% formic acid B: Acetonitrile/0.1% formic acid |

| Time | A % | B % |
|---|---|---|
| 0.00 | 90 | 10 |
| 2.50 | 30 | 70 |
| 3.00 | 5 | 95 |
| 4.50 | 5 | 95 |

UV spectra were recorded at 210 nm with a sampling rate of 1.0 spectra/second. Mass spectra were obtained using positive and negative electrospray ionization over the range m/z 100 to 700. The scan rate was 1.0 scan (m/z 100 to 700) per second.

### Synthesis of Compounds

First of all general methods will be presented for the synthesis of basic building blocks. In subsequent reactions these basic building blocks can be functionalized via common methods of organic syntheses to obtain the desired target compounds.

### General Method 1A:

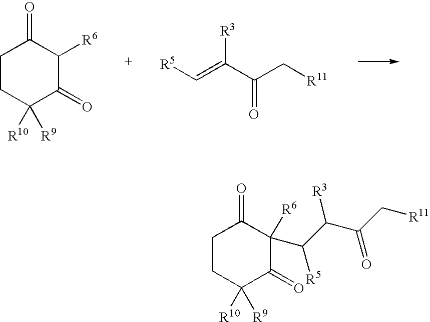

This scheme illustrates the synthesis of the first precursor, which ist the starting compound for the subsequent ring closure reaction to form various hydronaphthalen derivatives.

To a solution of the cyclohexane-1,3-dione in water the enone is added. Some ethanol is added to enhance the solubility and the developing suspension is stirred for 7 days at room temperature (after 6 days the white solid is completely dissolved). The solution is mixed with toluene and the aqueous phase is extracted with dichloromethane. The combined organic phases are washed with saturated NaCl solution and dried over MgSO₄. The solvent is removed under reduced pressure and the crude product is purified via column chromatography.

Alternatively, if the solubility capability of water is not sufficient, the following general method can be used. To a solution of the cyclohexane-1,3-dione, a 2.5 M solution of Triton B (benzyltrimethylammoniumhydroxide) in methanol the enone dissolved in methanol is added. The suspension is heated at gently elevated temperature for 5 h and stirred afterwards at room temperature over night. The solvent is removed under reduced pressure, toluene is added, the phases are separated and the organic phase is extracted with dichloromethane. The combined organic phases are washed with saturated NaCl solution and dried over MgSO₄. The solvent is removed under reduced pressure and the crude product is purified via column chromatography.

### General Method 1B:

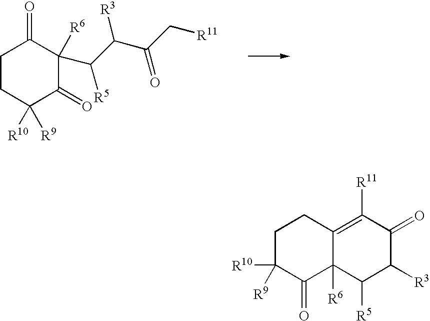

This scheme illustrates the above-mentioned ring closure reaction of the obtained compound according to the General Method 1A.

To a solution of said cyclohexane-1,3-dione in benzene pyrrolidine is added and the suspension is heated under reflux by means of a water separator for the azeotropic removing of the water. Afterwards the benzene and excess pyrrolidine are removed under reduced pressure. The obtained solid is dissolved in dichloromethane and water. The phases are separated and the aqueous phase is extracted with dichloromethane. The combined organic phases are primary washed with saturated NH₄Cl solution, then with saturated NaHCO₃ solution and finally with saturated NaCl solution. It is dried over MgSO₄, the solvent is removed under reduced pressure and the crude product is purified via column chromatography.

This ring closure reaction can be also carried out in a stereoselective manner.

### General Method 1C:

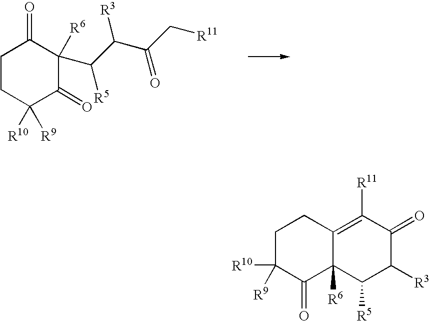

This scheme illustrates the above-mentioned ring closure reaction of the obtained compound according to General Method 1A with controlled stereoselectivity.

To a solution of said cyclohexane-1,3-dione in DMF L-phenylalanine and D-camphorsulfonic acid are added. The solution is stirred over night at room temperature and afterwards the temperature is increased every 24 h in each case by 10° C. until the final value of 70° C. The solution is cooled to 0° C. and mixed with a saturated NaHCO₃ solution. After some stirring it is extracted with diethylether. The combined organic phases are washed with saturated NaCl solution and dried over MgSO₄. The solvent is removed under reduced pressure and the crude product is purified via column chromatography.

This synthesis step takes place in all cases, which will be described more detailed in the Examples 1 to 5 below.

As next the conversion of the carbonyl group an the position 5 of the hydronaphthalen into a hydroxy group takes place. Via the obtained OH group the functionalization capability of these compounds is achieved.

### General Method 1D:

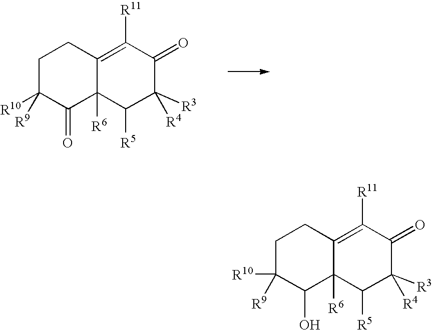

This scheme illustrates the conversion of the carbonyl group an the position 5.

To a solution of the desired compound in absolute ethanol a solution of sodium borohydride in ethanol is added dropwise at 0° C. within about 3 h. After some stirring the solution is admixed with pure acetic acid dropwise, until no gas evolution is observable. It is mixed with toluene and the solvent is removed under vacuum. The residue is taken up in chloroform, washed with saturated NaCl solution and dried over MgSO₄. The solvent is removed under reduced pressure and the crude product is purified via column chromatography.

In the following Examples 1 to 5 according to the General Method 1a detailed synthesis description is given. The compounds synthesized are obtained via the stereoselective way.

### Example 1

### Synthesis of (4aS,5S)-4,4a,5,6,7,8-hexahydro-5-hydroxy-1,4a-dimethylnaphthalen-2(3H)-one

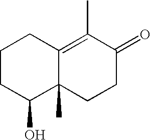

### Synthesis of 2-methyl-2-(3-oxopentyl)cyclohexane-1,3-dione

To a solution of 37.5 g (298 mmol) 2-methylcyclohexane-1,3-dione in 75 ml of water 50.0 ml (512 mmol) of pent-1-en-3-one is added. 2.5 ml of ethanol is added to enhance the solubility and the developing suspension is stirred for 7 days at room temperature (after 6 days the white solid is completely dissolved). The solution is mixed with 300 ml of toluene and the aqueous phase is extracted three times each with 300 ml of dichloromethane. The combined organic phases are washed with saturated NaCl solution and dried over MgSO₄. The solvent is removed under reduced pressure and the crude product is purified via column chromatography (colorless oil).

Yield: 53.7 g, 256 mmol, 86%

R_{f}-value: 0.16 (cyclohexane/ethylacetate 3:1 v/v)

¹H-NMR (400 MHz, CDCl₃): δ 1.02 (t, J=7.4 Hz, 3H), 1.24 (s, 3H), 1.98-2.10 (m, 2H), 2.2-2.58 (m, 10H).

GCMS, m/z (rel int. %): 210 (49) [M⁺], 192 (18), 181 (21), 153 (15), 139 (82), 111 (100), 97 (45), 69 (43), 57 (64).

### Synthesis of (S)-3,4,8,8a-tetrahydro-5,8a-dimethylnaphthalen-1,6(2H,7H)-dione

To a solution of 49.0 g (233 mmol) 2-methyl-2-(3-oxopentyl)cyclohexane-1,3-dione in 600 ml of DMF, 37.0 g (224 mmol) L-phenylalanine and 27.0 g (166 mmol) D-camphorsulfonic acid are added. The solution is stirred over night at room temperature and afterwards the temperature is increased every 24 h in each case by 10° C. until the final value of 70° C. The solution is cooled to 0° C. and mixed with 600 ml of a saturated NaHCO₃ solution. After 30 min of stirring it is extracted each time with 400 ml of diethylether. The combined organic phases are washed with saturated NaCl solution and dried over MgSO₄. The solvent is removed under reduced pressure and the crude product is purified via column chromatography (white solid).

Mp.: 46° C.

Yield: 32.0 g, 166.7 mmol, 71%

R_{f}-value: 0.24 (cyclohexane/ethylacetate 3:1 v/v)

[α]_{D}²⁰=+136° (c=1.00, CHCl₃)

¹H-NMR (400 MHz, CDCl₃): δ 1.41 (s, 3H), 1.80 (s, 3H), 2.02-2.21 (m, 4H), 2.38-2.59 (m, 4H), 2.81-2.94 (m, 1H).

GCMS, m/z (rel int. %): 192 (34) [M⁺], 177 (38), 149 (57), 136 (100), 121 (41), 107 (74), 93 (82), 79 (49), 55 (31), 39 (23).

### Synthesis of (4aS,5S)-4,4a,5,6,7,8-hexahydro-5-hydroxy-1,4a-dimethylnaphthalen-2(3H)-one

To a solution of 32.0 g (166.7 mmol) (S)-3,4,8,8a-tetrahydro-5,8a-dimethylnaphthalen-1,6(2H,7H)-dione in 250 ml of absolute ethanol a solution of 1.68 g (44.4 mmol) sodium borohydride in 70 ml of ethanol is added dropwise at 0° C. within 3 h. After 30 min of stirring the solution is admixed with pure acetic acid dropwise, until no gas evolution is observable. It is mixed with 50 ml of toluene and the solvent is removed under vacuum. The residue is taken up in 500 ml of chloroform, washed with saturated NaCl solution and dried over MgSO₄. The solvent is removed under reduced pressure and the crude product is purified via column chromatography (colorless oil).

Yield: 25.2 g, 129.9 mmol, 78%

R_{f}-value: 0.12 (cyclohexane/ethylacetate 3:1 v/v)

[α]_{D}²⁰=+161° (c=1.00, CHCl₃)

¹H-NMR (400 MHz, CDCl₃): δ 1.11 (s, 3H), 1.21-1.35 (m, 1H), 1.69 (s, 3H), 1.58-1.92 (m, 4H), 1.94-2.10 (m, 2H), 2.30-2.39 (m, 2H), 2.55-2.65 (m, 1H), 3.34 (dd, J=4.5, 7.2 Hz, 1H), 3.60 (brs, 1H).

¹³C-NMR (100.6 MHz, CDCl₃): δ 11.5, 16.0, 23.1, 27.3, 30.1, 33.6, 42.2, 78.3, 129.9, 161.8, 199.6.

GCMS, m/z (rel int. %): 194 (81) [M⁺], 179 (7), 151 (12), 138 (100), 123 (41), 110 (34), 91 (27), 77 (19), 67 (9), 55 (11) 41 (11).

### Example 2

### Synthesis of (4S,4aS,5S)-4,4a,5,6,7,8-hexahydro-5-hydroxy-4,4a-dimethylnaphthalen-2(3H)-one

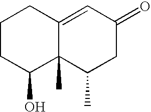

### Synthesis of (8S,8aS)-3,4,8,8a-tetrahydro-8,8a-dimethylnaphthalen-1,6(2H,7H)-dione

To a solution of 10.0 g (80.1 mmol) 2-methylcyclohexane-1,3-dione in 75 ml of water 7.4 g (88.2 mmol) of trans-pentene-2-one is added. 1.5 ml of ethanol is added to enhance the solubility and the developing suspension is stirred for 7 days at room temperature (after 6 days the white solid is completely dissolved). The solution is mixed with 200 ml of toluene and the aqueous phase is extracted three times each with 200 ml of dichloromethane. The combined organic phases are washed with saturated NaCl solution and dried over MgSO₄. The solvent is removed under reduced pressure.

The residue is admixed with 300 ml of DMF, 18.5 g (112 mmol) L-phenylalanine and 13.5 g (83.2 mmol) D-camphorsulfonic acid are added. The solution is stirred over night at room temperature and afterwards the temperature is increased every 24 h in each case by 10° C. until the final value of 70° C. The solution is cooled to 0° C. and mixed with 300 ml of a saturated NaHCO₃ solution. After 30 min of stirring it is extracted each time with 250 ml of diethylether. The combined organic phases are washed with saturated NaCl solution and dried over MgSO₄. The solvent is removed under reduced pressure and the crude product is purified via column chromatography (white solid).

Mp.: 58° C.

Yield: 9.49 g, 48.9 mmol, 61%

R_{f}-value: 0.22 (cyclohexane/ethylacetate 3:1 v/v)

[α]_{D}²⁰=+72° (c=1.00, CHCl₃)

¹H-NMR (400 MHz, CDCl₃): δ 0.78 (d, J=6.8 Hz, 3H), 1.36 (s, 3H), 1.52-1.65 (m, 1H), 1.94-2.03 (m, 1H), 2.09-2.15 (m, 2H), 2.31-2.38 (m, 1H), 2.43-2.67 (m, 4H), 5.85 (s, 1H).

¹³C-NMR (100.6 MHz, CDCl₃): δ 16.8, 20.8, 26.2, 32.0, 35.8, 39.0, 41.5, 54.6, 124.8, 164.2, 197.9, 211.8.

GCMS, m/z (rel int. %): 192 (15) [M⁺], 174 (17), 150 (22), 135 (100), 122 (11), 107 (13), 94 (16), 79 (16), 55 (13), 39 (11).

HRMS (FAB): calc. for C₁₂H₁₆O₂: 192.1150, found: 193.1221 [M+H]⁺.

### Synthesis of (4S,4aS,5S)-4,4a,5,6,7,8-hexahydro-5-hydroxy-4,4a-dimethylnaphthalen-2(3H)-one

To a solution of 15.9 g (82.8 mmol) (8S,8aS)-3,4,8,8a-tetrahydro-8,8a-dimethylnaphthalen-1,6(2H,7H)-dione in 150 ml of absolute ethanol a solution of 776 mg (20.5 mmol) sodium borohydride in 30 ml of ethanol is added dropwise at 0° C. within 3 h. After 30 min of stirring the solution is admixed with pure acetic acid dropwise, until no gas evolution is observable. It is mixed with 20 ml of toluene and the solvent is removed under vacuum. The residue is taken up in 300 ml of chloroform, washed with saturated NaCl solution and dried over MgSO₄. The solvent is removed under reduced pressure and the crude product is purified via column chromatography (colorless oil).

Yield: 12.7 g, 65.4 mmol, 79%

R_{f}-value: 0.12 (cyclohexane/ethylacetate 3:1 v/v)

[α]_{D}²⁰=+880 (c=1.00, CHCl₃)

¹H-NMR (400 MHz, CDCl₃): δ 0.79 (d, J=7.0 Hz, 3H), 1.26 (s, 3H), 1.20-1.40 (m, 1H), 1.40-1.59 (m, 1H), 1.98-2.04 (m, 2H), 2.11-2.18 (m, 1H), 2.43-2.67 (m, 6H), 6.07 (s, 1H).

¹³C-NMR (100.6 MHz, CDCl₃): δ 17.6, 18.6, 26.0, 31.6, 36.3, 37.9, 42.3, 47.6, 82.8, 127.8, 177.1, 197.9.

GCMS, m/z (rel int. %): 194 (31) [M⁺], 161 (100), 138 (54), 109 (26), 91 (31), 77 (18), 67 (11), 55 (10), 41 (13).

HRMS (FAB): calc. for C₁₂H₁₈O₂: 194.1307, found: 195.1392 [M+H]⁺.

### Example 3

### Synthesis of (4aR,5R)-4,4a,5,6,7,8-hexahydro-5-hydroxy-4a-methylnaphthalen-2(3H)-one

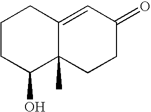

### Synthesis of (S)-3,4,8,8a-tetrahydro-8a-methylnaphthalen-1,6(2H,7H)-dione

To a solution of 12.6 g (100 mmol) 2-methylcyclohexane-1,3-dione, 4.4 ml (11.0 mmol) of a 2.5 M solution of Triton B (benzyltrimethylammoniumhydroxide) in methanol 14.6 ml (10.5 g, 150 mmol) of but-3-en-2-one is added in 60 ml of methanol. The suspension is heated at 60° C. for 5 h and stirred afterwards at room temperature over night. The solvent is removed under reduced pressure and the crude product is purified via column chromatography.

It is admixed with 300 ml of DMF, 23.3 g (141 mmol) L-phenylalanine and 17.0 g (105 mmol) D-camphorsulfonic acid are added. The solution is stirred over night at room temperature and afterwards the temperature is increased every 24 h in each case by 10° C. until the final value of 70° C. The solution is cooled to 0° C. and mixed with 300 ml of a saturated NaHCO₃ solution. After 30 min of stirring it is extracted each time with 250 ml of diethylether. The combined organic phases are washed with saturated NaCl solution and dried over MgSO₄. The solvent is removed under reduced pressure and the crude product is purified via column chromatography (colorless oil).

Yield: 12.7 g, 65.4 mmol, 60%

R_{f}-value: 0.27 (cyclohexane/ethylacetate 3:1 v/v)

[α]_{D}²⁰=+103° (c=1.09, CHCl₃)

¹H-NMR (400 MHz, CDCl₃): δ 1.42 (s, 3H), 1.61-1.78 (m, 1H), 2.03-2.20 (m, 3H), 2.36-2.53 (m, 4H), 2.66-2.78 (m, 2H), 5.84 (s, 1H).

¹³C-NMR (100.6 MHz, CDCl₃): δ 23.0, 23.4, 29.7, 31.8, 33.7, 37.7, 50.6, 125.7, 165.6, 198.0, 210.7.

GCMS, m/z (rel int. %): 178 (29) [M⁺], 160 (49), 150 (16), 136 (41), 121 (100), 108 (76), 93 (83), 79 (91), 55 (34), 39 (27).

HRMS (FAB): calc. for C₁₁H₁₄O₂: 178.0994, found: 179.1085 [M+H]⁺.

### Synthesis of (4aR,5R)-4,4a,5,6,7,8-hexahydro-5-hydroxy-4a-methylnaphthalen-2(3H)-one

To a solution of 17.0 g (95.5 mmol) (S)-3,4,8,8a-tetrahydro-8a-methylnaphthalen-1,6(2H,7H)-dione in 150 ml of absolute ethanol a solution of 946 mg (25.0 mmol) sodium borohydride in 30 ml of ethanol is added dropwise at 0° C. within 3 h. After 30 min of stirring the solution is admixed with pure acetic acid dropwise, until no gas evolution is observable. It is mixed with 20 ml of toluene and the solvent is removed under vacuum. The residue is taken up in 300 ml of chloroform, washed with saturated NaCl solution and dried over MgSO₄. The solvent is removed under reduced pressure and the crude product is purified via column chromatography (colorless oil).

Yield: 13.9 g, 77.4 mmol, 81%

R_{f}-value: 0.12 (cyclohexane/ethylacetate 3:1 v/v)

[α]_{D}²⁰=+78° (c=1.00, CHCl₃)

¹H-NMR (400 MHz, CDCl₃): δ 1.17 (s, 3H), 1.22-1.53 (m, 6H), 1.80-2.78 (m, 5H), 3.31 (t, J=6.4 Hz, 1H), 5.71 (s, 1H).

¹³C-NMR (100.6 MHz, CDCl₃): δ 14.3, 15.5, 21.2, 27.4, 30.3, 32.3, 33.8, 41.7, 60.6, 125.4, 171.6, 200.2.

GCMS, m/z (rel int. %): 180 (44) [M⁺], 162 (10), 152 (12), 137 (16), 124 (100), 109 (69), 91 (31), 79 (35), 67 (23), 55 (28), 43 (35).

HRMS (FAB): calc. for C₁₁H₁₆O₂: 180.1150, found: 181.1217 [M+H]⁺.

### Example 4

### Synthesis of (4aR,5S)-4a-benzyl-4,4a,5,6,7,8-hexahydro-5-hydroxy-1-methylnaphthalen-2(3H)-one

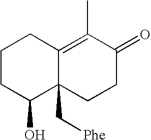

### Synthesis of 2-benzyl-2-(3-oxopentyl)cyclohexane-1,3-dione

To a solution of 20.2 g (100 mmol) 2-benzylcyclohexane-1,3-dione, 4.4 ml (11.0 mmol) of a 2.5 M solution of Triton B (benzyltrimethylammoniumhydroxide) in methanol 12.6 ml (12.6 g, 150 mmol) of pent-1-ene-3-one is added in 60 ml of methanol. The suspension is heated at 60° C. for 5 h and stirred afterwards at room temperature over night. The solvent is removed under reduced pressure, 400 ml of toluene is added, the phases are separated and the organic phase is extracted each time with 300 ml of dichloromethane. The combined organic phases are washed with saturated NaCl solution and dried over MgSO₄. The solvent is removed under reduced pressure and the crude product is purified via column chromatography (colorless oil).

Yield: 22.9 g, 80.1 mmol, 80%

R_{f}-value: 0.21 (cyclohexane/ethylacetate 3:1 v/v)

¹H-NMR (400 MHz, CDCl₃): δ 0.99 (t, J=7.0 Hz, 3H), 1.93-2.03 (m, 2H), 2.11-2.64 (m, 10H), 3.66 (s, 2H), 7.17-7.37 (m, 5H).

¹³C-NMR (100.6 MHz, CDCl₃): δ 7.9, 16.1, 21.4, 35.9, 36.9 (2C), 37.1, 38.5, 70.7, 127.2, 128.1 (2C), 128.7 (2C), 135.2, 200.0, 210.0, 210.4.

GCMS, m/z (rel int. %): 286 (32) [M⁺], 258 (12), 202 (13), 187 (31), 173 (56), 158 (42), 115 (27), 91 (100), 57 (21), 42 (10).

HRMS (FAB): calc. for C₁₈H₂₂O₃: 286.1569, found: 287.1655 [M+H]⁺.

### Synthesis of (R)-8a-benzyl-3,4,8,8a-tetrahydro-5-methylnaphthalen-1,6(2H,7H)-dione

To a solution of 20.0 g (69.9 mmol) 2-benzyl-2-(3-oxopentyl)cyclohexane-1,3-dione in 400 ml of DMF, 11.1 g (66.9 mmol) L-phenylalanine and 8.1 g (49.8 mmol) D-camphorsulfonic acid are added. The solution is stirred over night at room temperature and afterwards the temperature is increased every 24 h in each case by 10° C. until the final value of 70° C. The solution is cooled to 0° C. and mixed with 500 ml of a saturated NaHCO₃ solution. After 30 min of stirring it is extracted each time with 300 ml of diethylether. The combined organic phases are washed with saturated NaCl solution and dried over MgSO₄. The solvent is removed under reduced pressure and the crude product is purified via column chromatography (white solid).

Mp.: 67° C.

Yield: 13.3 g, 49.6 mmol, 71%

R_{f}-value: 0.12 (cyclohexane/ethylacetate 3:1 v/v)

[α]_{V}²⁰=+78° (c=1.00, CHCl₃)

¹H-NMR (400 MHz, CDCl₃): δ 1.65-1.75 (m, 1H), 1.86 (s, 3H), 1.87-1.91 (m, 1H), 1.91-2.02 (m, 2H), 2.07-2.12 (m, 1H), 2.36-2.52 (m, 5H), 2.60-2.71 (m, 1H), 2.83-2.91 (m, 1H), 6.97-7.03 (m, 1H), 7.24-7.30 (m, 2H), 7.37-7.42 (m, 2H).

¹³C-NMR (100.6 MHz, CDCl₃): δ 11.8, 21.5, 27.1, 29.3, 37.0, 38.9, 43.2, 56.1, 127.2, 128.7, 129.8 (4C), 141.2, 157.9, 197.7, 211.1.

GCMS, m/z (rel int. %): 268 (17) [M⁺], 176 (9), 141 (8), 115 (11), 91 (100), 77 (12), 65 (12).

HRMS (FAB): calc. for C₁₈H₂₀O₂: 268.1463, found: 269.1530 [M+H]⁺.

### Synthesis of (4aR,5S)-4a-benzyl-4,4a,5,6,7,8-hexahydro-5-hydroxy-1-methylnaphthalen-2(3H)

To a solution of 12.8 g (47.8 mmol) (R)-8a-benzyl-3,4,8,8a-tetrahydro-5-methylnaphthalen-1,6(2H,7H)-dione in 100 ml of absolute ethanol a solution of 473 mg (12.5 mmol) sodium borohydride in 20 ml of ethanol is added dropwise at 0° C. within 2.5 h. After 30 min of stirring the solution is admixed with pure acetic acid dropwise, until no gas evolution is observable. It is mixed with 20 ml of toluene and the solvent is removed under vacuum. The residue is taken up in 300 ml of chloroform, washed two times with saturated NaCl solution and dried over MgSO₄. The solvent is removed under reduced pressure and the crude product is purified via column chromatography (colorless oil).

Yield: 13.9 g, 77.4 mmol, 81%

R_{f}-value: 0.10 (cyclohexane/ethylacetate 3:1 v/v)

[α]_{D}²⁰=+72° (c=1.01, CHCl₃)

¹H-NMR (400 MHz, CDCl₃): δ 1.23-1.57 (m, 6H), 1.82 (s, 3H), 1.90-2.09 (m, 2H), 2.26-2.51 (m, 2H), 2.74-2.87 (m, 1H), 2.95-3.02 (m, 1H), 3.20-3.26 (m, 1H), 7.04-7.12 (m, 1H), 716-7.20 (m, 2H), 7.35-7.40 (m, 2H).

¹³C-NMR (100.6 MHz, CDCl₃): δ 11.7, 23.8, 27.7, 30.9, 31.1, 33.7, 36.7, 46.8, 80.1, 126.9, 129.9 (4C), 132.7, 138.6, 157.6, 199.2.

GCMS, m/z (rel int. %): 270 (41) [M⁺], 178 (35), 161 (100), 137 (16), 105 (22), 91 (76), 79 (25), 67 (13), 55 (18), 43 (25).

HRMS (FAB): calc. for C₁₈H₂₂O₂: 270.1620, found: 271.1712 [M+H]⁺.

### Example 5

### Synthesis of (4S,4aS,5S)-4a-allyl-4,4a,5,6,7,8-hexahydro-5-hydroxy-4-methylnaphthalen-2(3H)-one

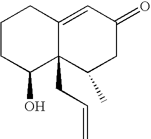

### Synthesis of 2-allylcyclohexane-1,3-dione

To a solution of 92.6 g (826 mmol) cyclohexane-1,3-dione in 100 ml of water and 200 ml (500 mmol) of a 2.5 M solution of Triton B (benzyltrimethylammoniumhydroxide) in methanol 69.9 ml (100 g, 826 mmol) of 3-bromoprop-1-en is added in 60 ml of methanol. The suspension is stirred at room temperature over night. The solvent is removed under reduced pressure, 400 ml of toluene is added, the phases are separated and the organic phase is extracted each time with 300 ml of dichloromethane. The combined organic phases are washed with saturated NaCl solution and dried over MgSO₄. The solvent is removed under reduced pressure and the crude product is purified via column chromatography (colorless oil).

Yield: 82.9 g, 545 mmol, 66%

R_{f}-value: 0.48 (cyclohexane/ethylacetate 3:1 v/v)

¹H-NMR (400 MHz, CDCl₃): δ 1.70-2.80 (m, 8H), 3.19 (t, J=6.9 Hz, 1H), 5.01-5.13 (m, 2H), 5.57-5.68 (m, 1H).

¹³C-NMR (100.6 MHz, CDCl₃): δ 15.2, 29.0, 40.9 (2C), 67.1, 116.4, 134.4, 208.3 (2C).

GCMS, m/z (rel int. %): 152 (41) [M⁺], 137 (61), 124 (49), 109 (14), 96 (100), 81 (34), 67 (21), 55 (44), 39 (23).

HRMS (FAB): calc. for C₉H₁₂O₂: 152.0837, found: 153.0912 [M+H]⁺.

### Synthesis of (8S,8aS)-8a-allyl-3,4,8,8a-tetrahydro-8-methylnaphthalen-1,6(2H,7H)-dione

To a solution of 13.4 g (88.1 mmol) 2-allylcyclohexane-1,3-dione in 75 ml of water 8.2 g (97.2 mmol) of pent-3-ene-2-one is added. 1.5 ml of ethanol is added to enhance the solubility and the developing suspension is stirred for 7 days at room temperature (after 6 days the white solid is completely dissolved). The solution is mixed with 200 ml of toluene and the aqueous phase is extracted three times each with 200 ml of dichloromethane. The combined organic phases are washed with saturated NaCl solution and dried over MgSO₄. The solvent is removed under reduced pressure and the crude product is purified via column chromatography.

Afterwards it is admixed with 400 ml of DMF, 24.8 g (150 mmol) L-phenylalanine and 18.1 g (111 mmol) D-camphorsulfonic acid are added. The solution is stirred over night at room temperature and afterwards the temperature is increased every 24 h in each case by 10° C. until the final value of 70° C. The solution is cooled to 0° C. and mixed with 400 ml of a saturated NaHCO₃ solution. After 30 min of stirring it is extracted each time with 300 ml of diethylether. The combined organic phases are washed with saturated NaCl solution and dried over MgSO₄. The solvent is removed under reduced pressure and the crude product is purified via column chromatography (white solid).

Mp.: 52° C.

Yield: 10.4 g, 47.7 mmol, 54%

R_{f}-value: 0.37 (cyclohexane/ethylacetate 3:1 v/v)

[α]_{D}²⁰=+54° (c=1.00, CHCl₃)

¹H-NMR (400 MHz, CDCl₃): δ 0.84 (d, J=7.6 Hz, 3H), 1.54-1.70 (m, 1H), 1.91-2.74 (m, 11H), 5.01-5.13 (m, 2H), 5.58-5.71 (m, 1H).

¹³C-NMR (100.6 MHz, CDCl₃): δ 17.0, 21.1, 33.2, 33.7, 40.3, 41.2, 43.8, 58.6, 119.3, 125.3, 132.7, 163.8, 197.9, 210.2.

GCMS, m/z (rel int. %): 218 (52) [M⁺], 203 (31), 190 (10), 175 (24), 161 (22), 149 (100), 135 (26), 119 (14), 105 (41), 91 (49), 77 (36), 55 (27), 41 (23).

HRMS (FAB): calc. for C₁₄H₁₈O₂: 218.1307, found: 219.1391 [M+H]⁺.

### Synthesis of (4S,4aS,5S)-4a-allyl-4,4a,5,6,7,8-hexahydro-5-hydroxy-4-methylnaphthalen-2(3H)-one

To a solution of 9.9 g (45.5 mmol) (8S,8aS)-8a-allyl-3,4,8,8a-tetrahydro-8-methylnaphthalen-1,6(2H,7H)-dione in 100 ml of absolute ethanol a solution of 483 mg (12.5 mmol) sodium borohydride in 20 ml of ethanol is added dropwise at 0° C. within 3 h. After 30 min of stirring the solution is admixed with pure acetic acid dropwise, until no gas evolution is observable. It is mixed with 20 ml of toluene and the solvent is removed under vacuum. The residue is taken up in 300 ml of chloroform, washed with saturated NaCl solution and dried over MgSO₄. The solvent is removed under reduced pressure and the crude product is purified via column chromatography (colorless oil).

Yield: 8.0 g, 36.3 mmol, 80%

R_{f}-value: 0.14 (cyclohexane/ethylacetate 3:1 v/v)

[α]_{D}²⁰=+660 (c=1.03, CHCl₃)

¹H-NMR (400 MHz, CDCl₃): δ 0.85 (d, J=8.0 Hz, 3H), 1.28-2.74 (m, 11H), 3.21-3.26 (m, 1H), 5.04-5.17 (m, 2H), 5.58-5.70 (m, 2H).

¹³C-NMR (100.6 MHz, CDCl₃): δ 16.9, 23.6, 28.5, 32.6, 35.4, 36.1, 44.7, 51.8, 79.5, 115.9, 123.8, 139.1, 170.0, 199.1.

GCMS, m/z (rel int. %): 220 (13) [M⁺], 202 (11), 179 (22), 161 (39), 137 (61), 119 (100), 105 (29), 91 (31), 77 (28), 65 (15), 55 (14), 41 (23).

HRMS (FAB): calc. for C₁₄H₂₀O₂: 220.1463, found: 221.1551 [M+H]⁺.

### General Method 2:

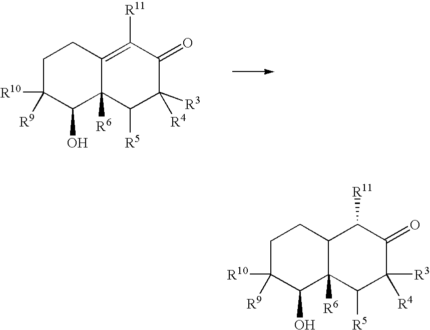

This scheme illustrates the hydrogenation reaction of the above described compounds. Said reaction can also be applied to compounds having other substituents than a hydroxy group in position 5. Normally, this reaction is enantioselective or diastereoselective, respectively.

To a solution of the compound to be hydrogenated in pyridine Pd/C are added. The obtained suspension is well stirred over night under a hydrogene atmosphere. Dichloromethane is added and the suspension is filtered over celite. The filtrate is primary washed with saturated NH₄Cl solution, then with saturated NaCl solution. The solvent is removed under reduced pressure and the crude product is dissolved in methanol. A solution of sodium mathanoate in methanol is added and the solution is stirred under reflux. After cooling to room temperature dichloromethane and water is added. The phases are separated and the aqueous phase is extracted with dichloromethane. The combined organic phases are washed with saturated NH₄Cl solution, then with saturated NaCl solution and dried over MgSO₄. The solvent is removed under reduced pressure and the crude product is purified via column chromatography.

### Example 6

### Synthesis of (1S,4aS,5S,8aR)-octahydro-5-hydroxy-1,4a-dimethylnaphthalen-2(1H)-one

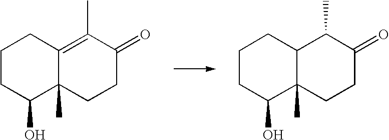

To a solution of 18.0 g (92.8 mmol) of (4aS,5S)-4,4a,5,6,7,8-hexahydro-5-hydroxy-1,4a-dimethylnaphthalen-2(3H)-one in 100 ml of pyridine 0.4 g Pd/C are added. The obtained suspension is well stirred over night under a hydrogene atmosphere. 250 ml dichloromethane are added and the suspension is filtered over celite. The filtrate is primary washed with saturated NH₄Cl solution, then with saturated NaCl solution. The solvent is removed under reduced pressure and the crude product is dissolved in 150 ml of methanol. 23.0 ml (124 mmol) of a 5.4 M solution of sodium mathanoate in methanol is added and the solution is stirred under reflux for 2 h. After cooling to room temperature 300 ml of dichloromethane and 300 ml of water is added. The phases are separated and the aqueous phase is extracted three times with dichloromethane. The combined organic phases are washed with saturated NH₄Cl solution, then with saturated NaCl solution and dried over MgSO₄. The solvent is removed under reduced pressure and the crude product is purified via column chromatography (colorless oil).

Yield: 11.2 g, 57.1 mmol, 62%

R_{f}-value: 0.15 (cyclohexane/ethylacetate 3:1 v/v)

[α]_{D}²⁰=−98° (c=1.00, CHCl₃)

¹H-NMR (400 MHz, CDCl₃): δ 0.96 (d, J=5.9 Hz, 3H), 1.07 (s, 3H), 1.17-1.85 (m, 9H), 2.19-2.55 (m, 4H), 3.22 (dd, J=4.5, 7.0 Hz, 1H).

¹³C-NMR (100.6 MHz, CDCl₃): δ 10.3, 11.7, 24.3, 25.1, 30.0, 34.0, 37.7, 39.2, 44.8, 50.8, 79.0, 213.0.

GCMS, m/z (rel int. %): 196 (26) [M⁺], 181 (21), 163 (17), 145 (10), 135 (51), 111 (100), 107 (32), 93 (12), 79 (9), 67 (11), 55 (10), 41 (9).

HRMS (FAB): calc. for C₁₂H₂₀O₂: 196.1463, found: 197.1558 [M+H]⁺.

### General Method 3:

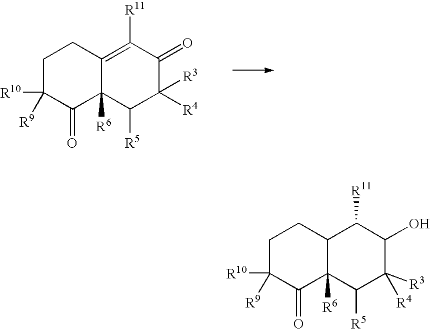

This scheme illustrates the hydrogenation reaction of the above described compounds and the additional conversion of the carbonyl group in position 2 to a hydroxyl group.

To a solution of the compound in dichloromethane 2-ethyl-2-methyl-1,3-dioxolan as well as catalytic amounts of ethyleneglycol and p-toluenesulfonic acid are added. The solution first stirred for 2 days at room temperature, then admixed with some ml of triethylamine and dichloromethane. The organic phase is washed with saturated NaCl solution and dried over MgSO₄. The solvent is removed under reduced pressure and the crude product is purified via column chromatography.

To a solution of the dioxolan protected compound in pyridine Pd/C are added. The obtained suspension is well stirred over night under a hydrogene atmosphere of 12 bar at elevated temperature for 3 days. Dichloromethane is added and the suspension is filtered over celite. The filtrate is primary washed with saturated NH₄Cl solution, then with saturated NaCl solution, finally it is dried over MgSO₄. The solvent is removed under reduced pressure and the crude product is dissolved in methanol. A 5.4 M solution of sodium mathanoate in methanol is added and the solution is stirred under reflux for 2 h. After cooling to room temperature dichloromethane and an equal amount of water is added. The phases are separated and the aqueous phase is extracted with dichloromethane. The combined organic phases are washed with saturated NH₄Cl solution, then with saturated NaCl solution and dried over MgSO₄. The solvent is removed under reduced pressure and the crude product is purified via column chromatography.

To a solution of the dioxolan protected hydrogenated compound in absolute diethylether a 1.0 M solution of diisobutylaluminiumhydride in toluene is added at −78° C. It is stirred at −78° C. for 1 h and then it is let to warm to room temperature. The solution is stirred at room temperature for some min, again cooled to −78° C. and quenched with saturated NH₄Cl solution. The solution is warmed to room temperature, the phases are separated and the aqueous phase is extracted with diethylether. The combined organic phases are washed with saturated NaCl solution and dried over MgSO₄. The solvent is removed under reduced pressure and the crude product is purified via column chromatography.

To remove the dioxolan protection group to a solution of the obtained compound in a 1:1 (v/v) mixture of acetone and water 2-hydroxy-pyridinium-(p-toluenesulfonate) is added and stirred at elevated temperature for 2 h. After cooling down to room temperature saturated NaHCO₃ solution is added, the phases are separated and the aqueous phase is extracted with dichloromethane. The combined organic phases are washed with saturated NaCl solution and dried over MgSO₄. The solvent is removed under reduced pressure and the crude product is purified via column chromatography.

### Example 7

### Synthesis of (5S,8aS)-octahydro-6-hydroxy-5,8a-dimethylnaphthalen-1(2H)-one

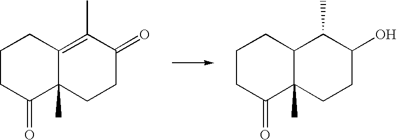

### Synthesis of (S)-4,4a,5,6,7,8-hexahydro-5-(1,3-dioxolan)-1,4a-dimethylnaphthalen-2(3H)-one

To a solution of 10.5 g (53.8 mmol) (S)-3,4,8,8a-tetrahydro-5,8a-dimethylnaphthalen-1,6(2H,7H)-dione in 100 ml of dichloromethane 100 ml of 2-ethyl-2-methyl-1,3-dioxolan as well as catalytic amounts of ethyleneglycol and p-toluenesulfonic acid are added. The solution is stirred for 2 days at room temperature, then admixed with 4 ml of triethylamine and 400 ml of dichloromethane. The organic phase is washed with saturated NaCl solution and dried over MgSO₄. The solvent is removed under reduced pressure and the crude product is purified via column chromatography (colorless oil).

Yield: 11.0 g, 48.4 mmol, 90%

R_{f}-value: 0.33 (cyclohexane/ethylacetate 3:1 v/v)

[α]_{D}²⁰=+125° (c=1.39, CHCl₃)

¹H-NMR (400 MHz, CDCl₃): δ 1.34 (s, 3H), 1.57-1.72 (m, 5H), 1.76 (d, J=1.2 Hz, 3H), 2.02-2.25 (m, 2H), 2.32-2.50 (m, 2H), 2.67-2.76 (m, 1H), 3.87-4.00 (m, 4H).

¹³C-NMR (100.6 MHz, CDCl₃): δ 11.5, 20.9, 21.4, 26.4, 26.5, 29.7, 33.7, 45.3, 65.0, 65.3, 112.8, 130.1, 156.0, 198.5.

GCMS, m/z (rel int. %): 236 (21) [M⁺], 180 (12), 149 (21), 121 (17), 107 (15), 99 (100), 91 (34), 77 (25), 55 (18), 41 (12).

HRMS (FAB): calc. for C₁₄H₂₀O₃: 236.1412, found: 237.1477 [M+H]⁺.

### Synthesis of (1S,4aS)-octahydro-5-(1,3-dioxolan)-1,4a-dimethylnaphthalen-2(1H)-one

To a solution of 2.79 g (11.8 mmol) of (S)-4,4a,5,6,7,8-hexahydro-5-(1,3-dioxolan)-1,4a-dimethylnaphthalen-2(3H)-one in 50 ml of pyridine 0.2 g Pd/C are added. The obtained suspension is well stirred over night under a hydrogene atmosphere of 12 bar at 65° C. for 3 days. 250 ml dichloromethane are added and the suspension is filtered over celite. The filtrate is primary washed with saturated NH₄Cl solution, then with saturated NaCl solution, finally it is dried over MgSO₄. The solvent is removed under reduced pressure and the crude product is dissolved in 20 ml of methanol. 2.3 ml (12.4 mmol) of a 5.4 M solution of sodium mathanoate in methanol is added and the solution is stirred under reflux for 2 h. After cooling to room temperature 200 ml of dichloromethane and 200 ml of water is added. The phases are separated and the aqueous phase is extracted two times with dichloromethane. The combined organic phases are washed with saturated NH₄Cl solution, then with saturated NaCl solution and dried over MgSO₄. The solvent is removed under reduced pressure and the crude product is purified via column chromatography (colorless oil).

Yield: 2.31 g, 9.7 mmol, 82%

R_{f}-value: 0.31 (cyclohexane/ethylacetate 3:1 v/v)

[α]_{D}²⁰=+83° (c=1.09, CHCl₃)

¹H-NMR (400 MHz, CDCl₃): δ 0.94 (d, J=6.6 Hz, 3H), 1.25 (s, 3H), 1.32-1.83 (m, 8H), 1.90-2.00 (m, 1H), 2.07-2.29 (m, 2H), 2.37-2.48 (m, 1H), 3.83-4.00 (m, 4H).

¹³C-NMR (100.6 MHz, CDCl₃): δ 12.4, 17.5, 22.6, 22.8, 25.9, 29.8, 38.2, 43.1, 44.1, 49.8, 65.4 (2C), 112.6, 213.4.

GCMS, m/z (rel int. %): 238 (17) [M⁺], 209 (31), 138 (11), 112 (74), 99 (100), 86 (78), 67 (14), 55 (21), 41 (16).

HRMS (FAB): calc. for C₁₄H₂₂O₃: 238.1569, found: 239.1659 [M+H]⁺.

### Synthesis of (1S,4aS)-decahydro-5-(1,3-dioxolan)-1,4a-dimethylnaphthalen-2-ol

To a solution of 3.41 g (14.3 mmol) (1S,4aS)-octahydro-5-(1,3-dioxolan)-1,4a-dimethylnaphthalen-2(1H)-one in 20 ml of absolute diethylether 14.5 ml (14.5 mmol) of a 1.0 M solution of diisobutylaluminiumhydride in toluene is added at −78° C. It is stirred at −78° C. for 1 h and then it is let to warm to room temperature. The solution is stirred at room temperature for 15 min, again cooled to −78° C. and quenched with 15 ml of saturated NH₄Cl solution. The solution is warmed to room temperature, the phases are separated and the aqueous phase is extracted two times with diethylether. The combined organic phases are washed with saturated NaCl solution and dried over MgSO₄. The solvent is removed under reduced pressure and the crude product is purified via column chromatography (colorless oil).

Yield: 3.05 g, 12.7 mmol, 89%

R_{f}-value: 0.33 (cyclohexane/ethylacetate 3:1 v/v)

[α]_{D}²⁰=+113° (c=1.39, CHCl₃)

¹H-NMR (400 MHz, CDCl₃): δ 0.92-1.00 (m, 3H), 1.12 (s, 3H), 1.24-1.83 (m, 12H), 2.31 (brs, 1H), 3.16 (m, 1H), 3.83-4.00 (m, 4H).

¹³C-NMR (100.6 MHz, CDCl₃): δ 12.7, 16.8, 20.9, 22.8, 23.6, 29.8, 32.0, 44.2, 44.3, 44.8, 65.4 (2C), 80.0, 112.8.

GCMS, m/z (rel int. %): 240 (11) [M⁺], 222 (13), 195 (10), 178 (12), 160 (16), 145 (15), 125 (26), 113 (22), 99 (100), 86 (71), 67 (10), 55 (18), 41 (12).

HRMS (FAB): calc. for C₁₄H₂₄O₃: 240.1725, found: 241.1817 [M+H]⁺.

### Synthesis of (5S,8aS)-octahydro-6-hydroxy-5,8a-dimethylnaphthalen-1(2H)-one

To a solution of 5.30 g (22.1 mmol) (1S,4aS)-decahydro-5-(1,3-dioxolan)-1,4a-dimethylnaphthalen-2-ol in 100 ml of a 1:1 (v/v) mixture of acetone and water 15.0 g (56.1 mmol) of 2-hydroxy-pyridinium-(p-toluenesulfonate) are added and stirred at 70° C. for 2 h. After cooling down to room temperature 400 ml of a saturated NaHCO₃ solution are added, the phases are separated and the aqueous phase is extracted three times with 500 ml of dichloromethane. The combined organic phases are washed with saturated NaCl solution and dried over MgSO₄. The solvent is removed under reduced pressure and the crude product is purified via column chromatography (colorless oil).

Yield: 3.51 g, 18.1 mmol, 82%

R_{f}-value: 0.33 (cyclohexane/ethylacetate 3:1 v/v)

[α]_{D}²⁰=+125° (c=1.39, CHCl₃)

¹H-NMR (400 MHz, CDCl₃): δ 0.97 (d, J=6.4 Hz, 3H), 1.27 (s, 3H), 1.38-2.05 (m, 10H), 2.12-2.31 (m, 2H), 3.10-3.17 (m, 1H).

¹³C-NMR (100.6 MHz, CDCl₃): δ 11.0, 21.4, 23.1, 27.2, 28.7, 35.6, 39.0, 45.2, 45.4, 55.2, 79.5, 214.3.

GCMS, m/z (rel int. %): 196 (32) [M⁺], 178 (81), 163 (31), 135 (100), 111 (75), 107 (71), 95 (62), 81 (66), 67 (69), 55 (73), 41 (78).

HRMS (FAB): calc. for C₁₂H₂₀O₂: 196.1463, found: 196.1481 [M]⁺.

In the subsequent General Methods various syntheses of organic chemistry will be described for solid phase reactions. These comprise amongst others the aldol, Sonogashira, reductive amination, amination of aryliodides, alkylation, Wittig, cleavage of silyl protection groups, and the Heck reaction, which can be also carried out in liquid phase. But it is stressed that these reactions are not limited to solid phase syntheses.

First basic methods for the loading of the polymer substrate as well as for the cleavage from the polymer substrate will be described.

Functionalization of the Merrifield Resin with the THP Linker

To a solution of 21.9 g (192 mmol) (3,4-dihydro-2H-pyran-2-yl)methanol in 150 ml of absolute THF 7.72 g (193 mmol) sodium hydride (60 percent in mass, in petroleum) are added and the suspension is shaked at room temperature over night. The solvent is removed under reduced pressure, afterwards 300 ml of N,N-dimethylacetamide are added and the solution is shaked for 5 min at room temperature. Afterwards 25.0 g (42.5 mmol, load ratio: 1.7 mmol/g) of Merrifield resin is added slowly, such that no clots are formed. The mixture is shaked for 2 days at room temperature and the resin is washed three times each with methanol, dichloromethane, dichloromethane:methanol=1:1 (v/v), methanol, and dichloromethane. Finally the resin is dried under vacuum.

### Determination of the Load Ratio

For determining the load ratio 300 mg of the dried resin are mixed with 20 ml of dichloromethane and shaked for 15 min. Afterwards 495 mg (2.55 mmol) of (4aS,5S)-4,4a,5,6,7,8-hexahydro-5-hydroxy-1,4a-dimethylnaphthalen-2(3H)-one as well as 48 mg (0.255 mmol) p-toluenesulfonic acid monohydrate are added and it is shaked over night. The solvent is filtered off and the resin is washed three times each with dichloromethane, dichloromethane:methanol=1:1 (v/v), methanol, dichloromethane:methanol=1:1 (v/v), and dichloromethane. Finally the resin is dried under vacuum. Afterwards 10 ml of a 10% solution of trifluoroacetic acid in dichloromethane are added and it is shaked for 10 min. The solvent is filtered off and the resin is shaked again ten times with 10 ml of the trifluoroacetic acid solution. 3 ml of toluene are added to the combined filtrates and the solvent is removed under reduced pressure.

Yield: 64.6 mg, 0.33 mmol, 65%, load ratio: 1.10 mmol/g

[α]_{D}²⁰=+1610 (c=1.06, CHCl₃)

¹H-NMR (400 MHz, CDCl₃): δ 1.11 (s, 3H), 1.21-1.35 (m, 1H), 1.69 (s, 3H), 1.58-1.92 (m, 4H), 1.94-2.10 (m, 2H), 2.30-2.39 (m, 2H), 2.55-2.65 (m, 1H), 3.34 (dd, J=4.5, 7.2 Hz, 1H), 3.60 (brs, 1H).

### Loading the Polymer Substrate (Resin):

1.0 eq. of THP-functionalized Merrifield resin are shaked in dichloromethane (15 ml/g) for 15 min. Afterwards 5 eq. of the alcohol to be coupled as well as 0.5 eq. of p-toluolsulfonic acid monohydrate are added, the mixture is shaked over night. The solvent is filtered off and the resin is washed three times each with dichloromethane, dichloromethane:methanol=1:1 (v/v), methanol, dichloromethane:methanol=1:1 (v/v) and dichloromethane. Finally the resin is dried under vacuum.

For determining the load ratio 30 mg of the dried resin are mixed with 2 ml of dichloromethane and it is shaked for 15 min. Afterwards 5 ml of a 10% solution of trifluoroacetic acid in dichloromethane is added and it is shaked for 10 min. The solvent is filtered off and the resin is shaked two times with 5 ml of the trifluoroacetic acid solution. To the combined filtrates 3 ml of toluene are added and the solvent is removed under reduced pressure. Amount and purity of the cleaved alcohol represent the load ration of the resin.

Cleavage from Polymer Substrate (Resin):

30 mg of the dried resin are added with 2 ml dichloromethane under argon and it is shaked for 15 min. Afterwards the solvent is filtered off and 3 ml of a 10% solution of trifluoroacetic acid in dichloromethane (v/v) is added and it is shaked for 10 min. The solvent is filtered off and the resin is shaked two times with 3 ml of the trifluoroacetic acid solution. To the combined filtrates 3 ml of toluene are added and the solvent is removed under reduced pressure. The obtained product is dissolved in 100 μl of acetonitrile and purified over a HPLC column (flow rate: 27 ml/min; CH₃CN/H₂O/TFA:10/90/0.1 (0 min), 30/70/0.1 (2 min), to 60/40/0.1 (15 min), to 80/20/0.1 (25 min), to 100/0/0.1 (26 min).

### General Method 4:

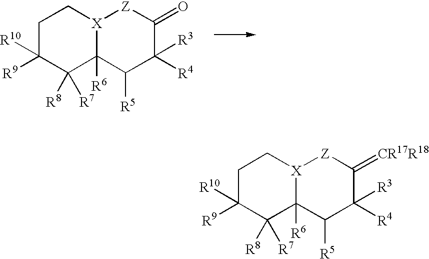

General method for the Wittig reaction at the solid phase.

The Wittig reaction allows the synthesis of alkenes via the reaction of aldehydes or ketones with ylides. The ylides are generated in situ by deprotonation of a phosphonium salt with a suitable base (e.g. NaH, NaOMe, NEt₃).

The E/Z selectivity of the Wittig reaction depends substantially from the stability of the used ylide. Stabilized ylides lead predominantly to (E)-alkenes, whereas non stabilized ylides lead to (Z)-alkenes.

1 eq. of the polymer bound ketone is admixed with toluene (2 ml/g resin) and it is shaked for 15 min. Afterwards 10 eq. of the corresponding triphenylphosphonium bromine and 8 eq. of butyllithium in toluene are added at room temperature. The solution is shaked for 15 min at room temperature and at 100° C. over night. The solvent is filtered off and the resin is washed three times each with toluene, dichloromethane:toluene=1:1 (v/v), dichloromethane:methanol=1:1 (v/v), methanol, dichloromethane:methanol=1:1 (v/v) and dichloromethane. Afterwards the resin is dried under vacuum.

### Example 8

### Synthesis of (1S,6Z,8aS)-6-ethylidene-1,2,3,4,6,7,8,8a-octahydro-5,8a-dimethylnaphthalen-1-ol

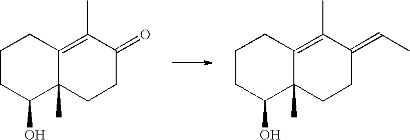

50 mg (55 μmol) of polymer bound (4aS,5S)-4,4a,5,6,7,8-hexahydro-5-hydroxy-1,4a-dimethylnaphthalen-2(3H)-one are converted with 203 mg (0.55 mmol) of ethyl-triphenylphosphoniumbromide and 0.28 ml (0.44 mmol) of a 1.6 M solution of butyllithium in toluene. The cleavage from the polymer substrate is as described above (colorless oil).

Yield: 5.1 mg, 25 μmol, 46%

[α]_{D}²⁰=+46° (c=1.07, CHCl₃)

E/Z ratio: 1:2.1

¹H-NMR (400 MHz, CDCl₃): δ 1.20 (s, 3H), 1.11-2.07 (m, 13H), 1.59 (s, 3H), 3.10 (brs, 1H), 3.37-3.45 (m, 1H), 5.30-5.38 (m, 1H).

¹³C-NMR (100.6 MHz, CDCl₃): δ 13.9, 14.7, 19.7, 24.9, 25.1, 28.6, 32.2, 40.4, 45.1, 86.7, 124.1, 135.9, 141.4, 152.3.

HRMS (FAB): calc. for C₁₄H₂₂O: 206.1671, found: 206.1678 [M]⁺.

### General Method 5:

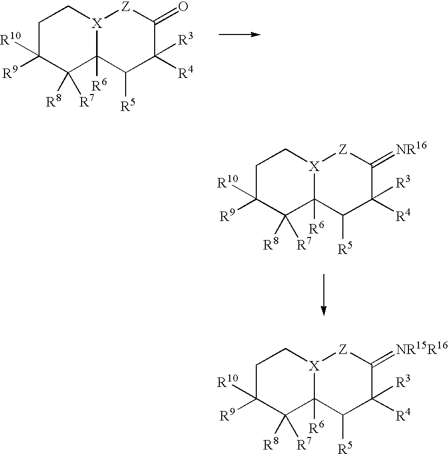

General method for the Leuckart-Wallach reaction at the solid phase.

This conversion is an important possibility for the synthesis of amines. Starting with a primary or secondary amine, alkylated amines can be obtained from carbonyl compounds (aldehydes or ketones) by means of formic acid as reducing agent. This reductive carbonyl-amination leads to the same result as the catalytic reductive amination of aldehydes and ketones, however under substantially easier conditions (autoclave not necessary) and with the advantage, that also compounds can be used, which normally would contaminate the hydrogenation catalyst.

In this variant of the Leuckart-Wallach reaction sodium cyanoborohydride is used instead of formic acid as reducing agent.

1 eq. of the dried resin is added with toluene (5 ml/g resin), 1.6 eq. of titan(IV)-chloride in toluene and 3.8 eq. of the corresponding amine under argon. Afterwards it is shaked over night at 90° C. The solvent is filtered off and the resin is washed three times each with toluene, dichloromethane: methanol=1:1 (v/v), methanol, dichloromethane:methanol=1:1 (v/v), and dichloromethane. Afterwards the resin is dried under vacuum and admixed with THF (3 ml/g resin) and 1.5 eq. of sodium cyanoborohydride. It is stirred at room temperature over night. The solvent is filtered off and the resin is washed three times each with THF, dichloromethane: methanol=1:1 (v/v), methanol, dichloromethane:methanol=1:1 (v/v), and dichloromethane. Finally the resin is dried under vacuum.

### General Method 6:

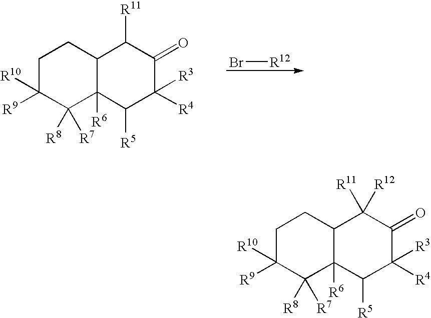

General method for the alkylation reaction at the solid phase.

To 1 eq. of the resin, which was initially swelled two times with dry THF for 15 min and washed, 5.0 eq. of potassium hydride in THF (3 ml/mmol) are added at room temperature. It is shaked for 30 min at room temperature, 8 eq. of the corresponding bromide are added and it is shaked for 1 h at room temperature as well as for 2 h at 50° C. The solvent is filtered off and the resin is washed three times each with THF, dichloromethane:THF=1:1 (v/v), methanol, dichloromethane:methanol=1:1 (v/v), and dichloromethane. Finally the resin is dried under vacuum.

### Example 9

### Synthesis of (4aS,5S)-1-butyl-octahydro-5-hydroxy-1,4a-dimethylnaphthalen-2(1H)-one

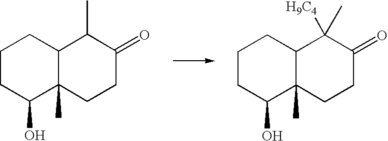

99 mg (109 μmol) polymer bound (4aS,5S)-octahydro-5-hydroxy-1,4a-dimethylnaphthalen-2(1H)-one are reacted according to said method with 93 μl (0.87 mmol) 1-bromobutane. The cleavage from the polymer substrate and the purification are carried out as described above (yellow oil).

Yield: 18.1 mg, 72 μmol, 66%

¹H-NMR (400 MHz, CDCl₃): δ 0.97 (t, J=6.1 Hz, 3H), 1.04 (s, 3H), 1.11 (s, 3H), 1.20-1.95 (m, 15H), 2.16-2.28 (m, 2H), 2.74 (brs, 1H), 3.13-3.17 (m, 1H).

### General Method 7 and 8:

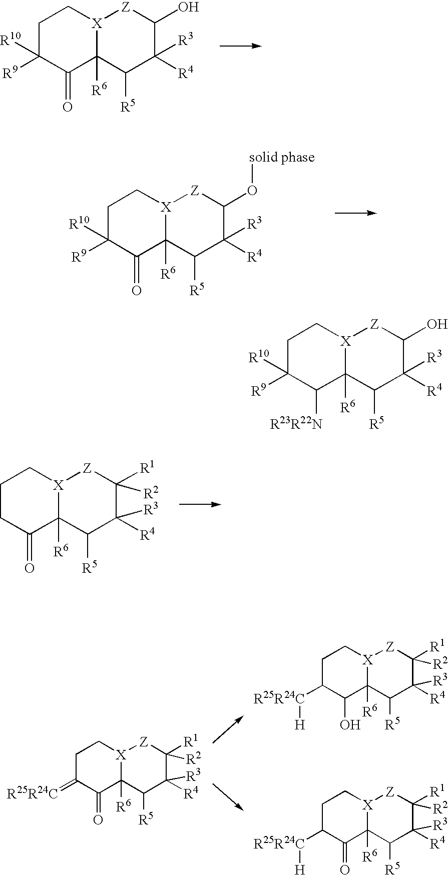

The illustrated general method 7 comprises contrary to the general method 1 the conversion of the carbonyl group at the position 2 of the hydronaphthalen into a hydroxy group. Via the obtained OH group the functionalization of these compounds is achieved on the opposite side of that molecule, i.e. at position 5. This is carried out according to the general method 3 which is then followed by the general method 5. The hydroxy group at position 2 can be protected or linked to a solid phase and the chemical reactions described herein can be applied to the carbonyl group at position 5.

### General Method 9:

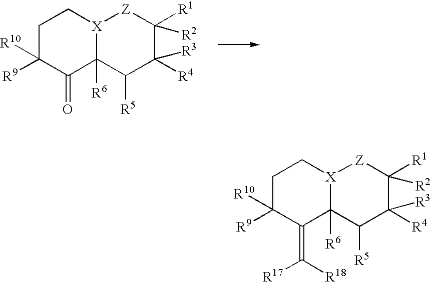

General method for the Wittig reaction at the solid phase according to general method 4. Contrary to that method the conversion of the carbonyl group herein takes place in the 5 position. In the present case, the substituents R¹ and R² do not form together a carbonyl group. R¹ and R² may form a carbonyl protecting group such as a dioxolane or a ketal or one of R¹ and R² represents a hydroxy group which is bound to a solid phase or optionally protected by a hydroxy protecting group.

### General Method 10:

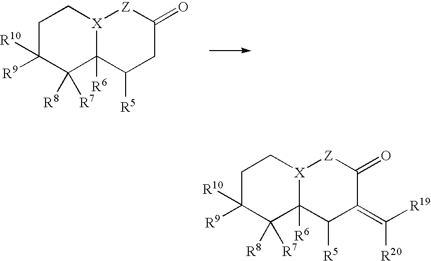

General method for the aldol reaction at the solid phase.

In this conversion generally referred to as aldol reaction enolates of aldehydes, ketones, esters or amides react as nucleophiles with other carbonyl groups as electrophiles. The enolate is generated from the C,H-acidic carbonyl compound with a base.

To a solution of 10 eq. Diisopropylamine in THF (1 ml/mmol) 9 eq. of butyllithium in hexane are added at −78° C. After the solution is warmed to room temperature it is added to 1.0 eq. of the loaded resin, which was initially swelled two times with dry THF for 15 min and washed. It is shaked at room temperature for 30 min and afterwards it is cooled to 0° C. 12 eq. of the corresponding aldehyde are added, whereupon it is shaked for 30 min at 0° C. and for 2 h at room temperature. The solvent is filtered off and the resin is washed three times each with THF, dichloromethane: methanol=1:1 (v/v), methanol, dichloromethane:methanol=1:1 (v/v), and dichloromethane. Finally the resin is dried under vacuum.

### Example 10

### Synthesis of (3E,4aS,5S)-3-benzylidene-4,4a,5,6,7,8-hexahydro-5-hydroxy-1,4a-dimethylnaphthalen-2(3H)-one (Compound No. 18)

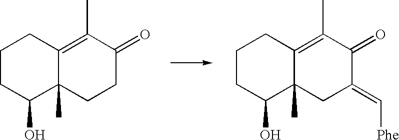

50 mg (55 μmol) of polymer bound (4aS,5S)-4,4a,5,6,7,8-hexahydro-5-hydroxy-1,4a-dimethylnaphthalen-2(3H)-one are reacted according to this method with 0.10 ml (0.73 mmol) of diisopropylamine, 0.27 ml (0.66 mmol) of a 2.5 M solution of butyllithium in hexane and 0.107 ml (1.06 mmol) freshly distilled benzaldehyde.

### General Method 11:

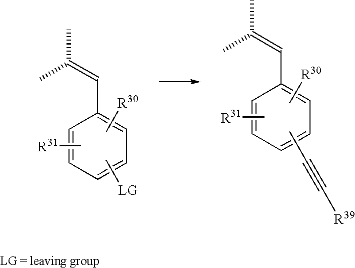

According to general method 10 the obtained compound can be further modified via the Sonogashira reaction at the solid phase.

This reaction allows the coupling of terminal alkines with aryl or vinyl halogenides by means of palladium-copper catalysis and under use of an amine as base. For the coupling of ethine, even in the case of an ethine excess, a trimethylsilyl protection group has to be introduced due to the double sided reaction.

1.0 eq. of the dried resin is admixed under argon with 1.0 eq. of CuI, 0.5 eq. of Pd(PPh₃)₄, DMF (15 ml/g resin), 20 eq. of diisopropylethylamine and 15 eq. of the corresponding terminal alkine. Afterwards it is shaked at 90° C. over night. The solvent is filtered off and the resin is washed three times each with THF:H₂O=2:1 (v/v), dichloromethane:methanol=1:1 (v/v), methanol, dichloromethane methanol=1:1 (v/v), and dichloromethane. Finally the resin is dried under vacuum.

### General Method 12:

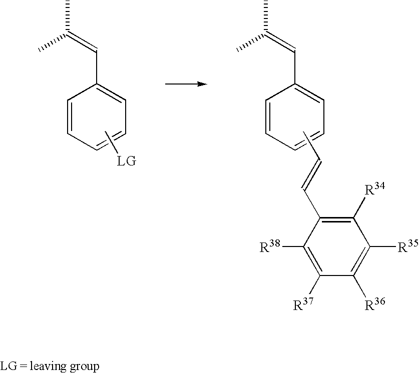

According to general method 10 the obtained compound can be further modified via the Heck reaction at the solid phase.

The Heck reaction allows the C—C coupling between olefins and aryl or vinyl halogenides under pallasium catalysis.

1.0 eq of the dried resin is swelled under argon with DMF (10 ml/g) for 15 min. Afterwards 5.0 eq. of olefin, 0.2 eq. of Pd(OAc)₂, 2.0 eq. of PPh₃ and 1.5 eq. of tetramethylethylendiamine are added. The suspension is shaked at 70° C. over night, the solvent is filtered off and the resin is washed three times each with THF:H₂O=2:1 (v/v), dichloromethane:methanol=1:1 (v/v), methanol, dichloromethane:methanol=1:1 (v/v), and dichloromethane. Finally the resin is dried under vacuum.

### Example 11

### Synthesis of (3E,4aS,5S)-3-(4-styrylbenzylidene)-4,4a,5,6,7,8-hexahydro-5-hydroxy-1,4a-dimethylnaphthalen-2(3H)-one

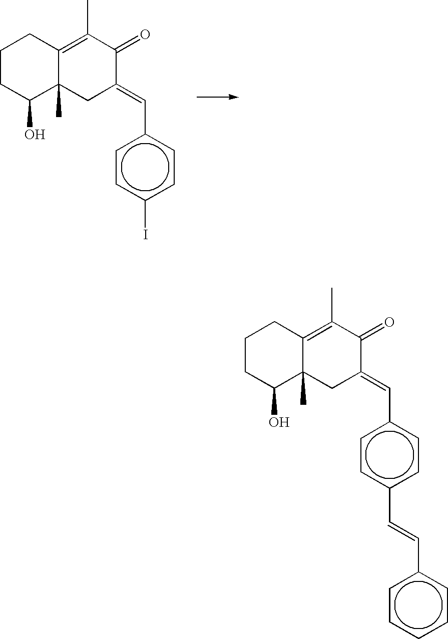

30 mg of polymer bound (3E,4aS,5S)-3-(4-iodobenzylidene)-4,4a,5,6,7,8-hexahydro-5-hydroxy-1,4a-dimethylnaphthalen-2(3H)-one (Compound No. 69) are swelled according to this method in 2 ml of DMF under argon. Afterwards 17 μl (0.165 mmol) of styrene, about 1 mg (4.5 μmol) of Pd(OAc)₂, 17.3 mg (66 μmol) of PPh₃ and 3.8 mg (33 μmol) of tetramethylethylenediamine are added. The suspension is shaked at 70° C. over night, the solvent is filtered off and the resin is washed three times each with THF: H₂O=2:1 (v/v), dichloromethane: methanol=1:1 (v/v), methanol, dichloromethane:methanol=1:1 (v/v), and dichloromethane. Finally the resin is dried under vacuum and it is cleaved and purified as described above (yellow oil).

Yield: 9.1 mg, 24 μmol, 72%

R_{f}-value: 0.14 (cyclohexane/ethylacetate 3:1 v/v)

[α]_{D}²⁰=+60° (c=1.05, CHCl₃)

¹H-NMR (400 MHz, CDCl₃): δ 1.24 (s, 3H), 1.27-1.68 (m, 4H), 1.85 (s, 3H), 2.04-2.83 (m, 4H), 3.23 (t, J=7.2 Hz, 1H), 7.10 (d, J=16.3 Hz, 1H), 7.15 (d, J=16.3 Hz, 1H), 7.22-7.45 (m, 10H).

### General Method 13:

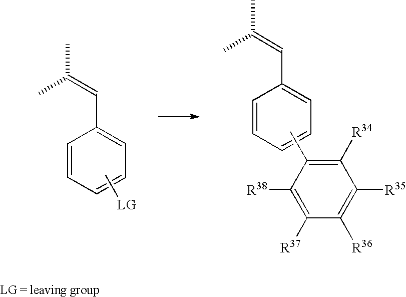

According to general method 10 the obtained compound can be further modified via the Suzuki reaction at the solid phase.

The Suzuki reaction is a palladium catalyzed coupling between organo boronic acids and aryl halogenids.

1.0 eq. of the dried resin is swelled under argon with THF (10 ml/g) for 15 min. Afterwards 2.0 eq. of Na₂CO₃, 0.5 eq. Pd(PPh₃)₄ and 2.0 eq. of boronic acid are added. The suspension is shaked at 60° C. over night, the solvent is filtered off and the resin is washed three times each with THF:H₂O=2:1 (v/v), dichloromethane:methanol=1:1 (v/v), methanol, dichloromethane:methanol=1:1 (v/v), and dichloromethane. Finally the resin is dried under vacuum.

### Example 12

### Synthesis of (3E,4aS,5S)-3-(4-phenylbenzylidene)-4,4a,5,6,7,8-hexahydro-5-hydroxy-1,4a-dimethylnaphthalen-2(3H)-one

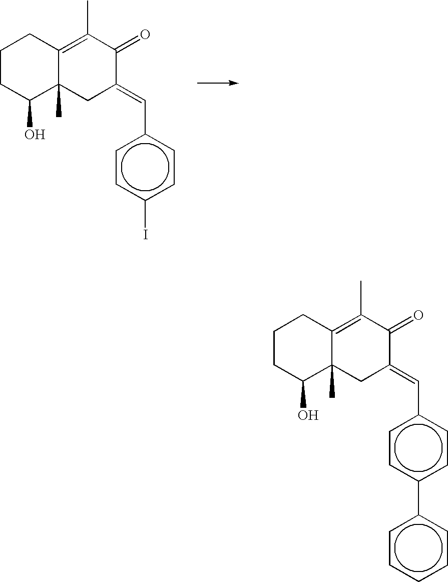

30 mg of polymer bound (3E,4aS,5S)-3-(4-iodobenzylidene)-4,4a,5,6,7,8-hexahydro-5-hydroxy-1,4a-dimethylnaphthalen-2(3H)-one (Compound No. 69) are swelled according to this method in 2 ml of THF for 15 min under argon. Afterwards 7 mg (66 μmol) of dry Na₂CO₃, 11 mg (16 μmol) of Pd(PPh₃)₄ and 8 mg (66 μmol) of phenylboronic acid are added. The suspension is shaked at 60° C. over night, the solvent is filtered off and the resin is washed three times each with THF:H₂O=2:1 (v/v), dichloromethane:methanol=1:1 (v/v), methanol, dichloromethane:methanol=1:1 (v/v), and dichloromethane. Finally the resin is dried under vacuum and it is cleaved and purified as described above (yellow oil).

Yield: 7.7 mg, 21.5 μmol, 65%

R_{f}-Wert: 0.19 (cyclohexane/ethylacetate 3:1 v/v)

[α]_{D}²⁰=+41° (c=1.02, CHCl₃)

¹H-NMR (400 MHz, CDCl₃): δ 1.22 (t, J=6.7 Hz, 3H), 1.23-2.14 (m, 8H), 1.95 (s, 3H), 3.23 (t, J=7.5 Hz, 1H), 7.15-7.54 (m, 10H).

### Materials and Methods:

Assay protocols for Cdc25A, 11βHSD1, 11βHSD2, and AChE are described in detain in the publication of Koch M A, Wittenberg L O, Basu S, Jeyaraj D A, Gourzoulidou E, Reinecke K, Odermatt A, Waldmann H (Proc. Natl. Acad. Sci. U.S.A. 2004 Nov. 30; 101(48):16721-6) and in Koch M A et al., Proc. Natl. Acad. Sci. U.S.A. 2005 Nov. 29; 102 (48):17272-7.

Assays protocols for the other enzymes mentioned herein (MtPTPA, PP1, VHR, PTP1B) are disclosed in Schweizer R A S, Atanasov, G A, Frey B M, and Odermatt A, Mol. Cell. Endocrinol. 2003, 212, 41-49 and Barratte, B., Meijer L., Galaktionov K. & Beach D, Anticancer Res. 1992, 12, 873-880. For determination of the phosphatase activity, a general phosphatase assay using para-nitrophenylphosphate was applied.

1. Compounds having the general formula (I): 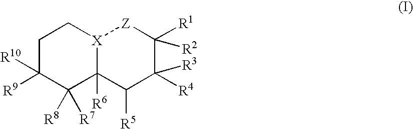
wherein
the moiety 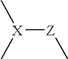
represents 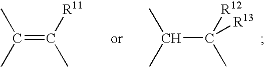
R¹ represents hydrogen and R² is —OH, —OR²¹, —NR¹⁴R¹⁵, or
R¹ and R² form together one of the residues ═O, ═NR¹⁶, or ═CR¹⁷R¹⁸;
R³ and R⁴ are hydrogen or form together the residue ═CR¹⁹R²⁰;
R⁵ and R⁶ represent independently of each other linear or branched, substituted or unsubstituted C₁-C₆-alkyl, linear or branched, substituted or unsubstituted C₂-C₆-alkenyl, —H, -Ph, —CH₂-Ph;
R⁷ represents hydrogen and R⁸ is one of —OH, —OR²¹, —NR²²R²³, or
R⁷ and R⁸ form together the residues ═O;
R⁹ and R¹⁰ are hydrogen or form together the residue ═CR²⁴R²⁵;
R¹¹, R¹², R¹³ represent independently of each other —H, linear or branched, substituted or unsubstituted C₁-C₆-alkyl, —CF₃, —CH₂—CO—O-(linear or branched, substituted or unsubstituted C₁-C₆-alkyl) or 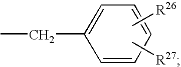
R¹⁴ and R¹⁵ represent independently of each other —H, linear or branched, substituted or unsubstituted C₁-C₆-alkyl, substituted or unsubstituted C₁-C₁₀-cycloalkyl, or 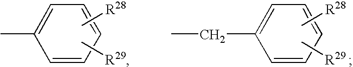
R¹⁶ represents —H or linear or branched, substituted or unsubstituted C₁-C₆-alkyl;
R¹⁷ and R¹⁸ represent independently of each other —H, linear or branched, substituted or unsubstituted C₁-C₆-alkyl, —CO—O-(linear or branched, substituted or unsubstituted C₁-C₆-alkyl), -Ph;
R¹⁹ and R²⁰ represent independently of each other —H, linear or branched, substituted or unsubstituted C₁-C₂₀-alkyl, substituted or unsubstituted heterocyclyl, substituted or unsubstituted aryl, substituted or unsubstituted heteroaryl, substituted or unsubstituted C₁-C₁₀-cycloalkyl 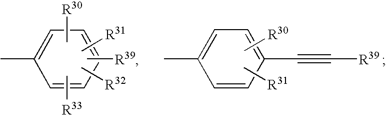
R²¹ represents —CF₃, linear or branched, substituted or unsubstituted C₁-C₂₀-alkyl;
R²² and R²³ represent independently of each other —H, linear or branched, substituted or unsubstituted C₁-C₂₀-alkyl, substituted or unsubstituted C₁-C₁₀-cycloalkyl, —CH₂-Ph;
R²⁴ and R²⁵ represent independently of each other —H, substituted or unsubstituted aryl, substituted or unsubstituted heteroaryl, linear or branched, substituted or unsubstituted C₁-C₂₀-alkyl;
R²⁶-R³⁸ represent independently of each other linear or branched, substituted or unsubstituted C₁-C₂₀-alkyl, —H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NH₂, NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NH-cyclo-C₃H₅, —NH—CS—NHC₃H₇, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N (cyclo-C₃H₅)₂, —NH—CS—N[CH(CH₃)₂]₂, —NH—CS—N[C(CH₃)₃]₂, —NH—C(═NH)—NH₂, —NH—C(═NH)—NHCH₃, —NH—C(═NH)—NHC₂H₅, —NH—CS—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —NH—CS—NHCH₃, —NH—C(═NH)—N(CH₃)₂, —NH—C(═NH)—N(C₂H₅)₂, —NH—C(═NH)—N(C₃H₇)₂, —NH—C(═NH)—N (cyclo-C₃H₅)₂, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F—CHF₂, —CF₃, —CH₂Cl, —CHCl₂, —CCl₃, —CH₂Br —CHBr₂, —CBr₃, —CH₂₁—CH₁₂, —Cl₃, —CPh₃, —CH₂—CH₂F—CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₂—CH₂I—CH₂—CH 2, —CH₂—Cl₃, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, -Ph, —CH₂-Ph, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C═CH;
R³⁹ represents R³⁷ or 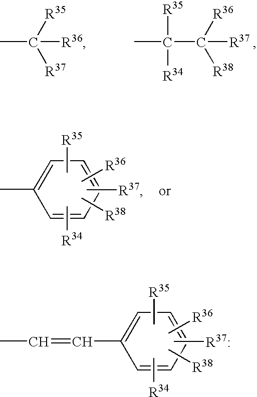
and stereoisomeric forms, prodrugs, solvates, hydrates and/or pharmaceutically acceptable salts thereof.

2. Compound according to claim 1 having the general formula IIB, 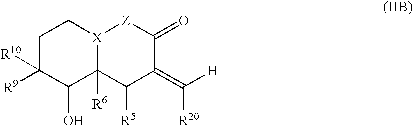
wherein
R⁵, R⁶, R⁹, R¹⁰, R²⁹, X and Z have the meanings as defined in claim 1.

3. Compound according to claim 1 or 2, wherein the compound is selected from the group comprising:
(3E,4aS,5S)-3-(3,4-Difluorbenzyliden)-4,4a,5,6,7,8-hexahydro-5-hydroxy-1,4a-dimethyl-naphthalen-2(3H)-one,
(3E,4aS,5S)-3-(4-Hydroxybenzyliden)-4,4a,5,6,7,8-hexahydro-5-hydroxy-1,4a-dimethyl-naphthalen-2(3H)-one,
(3E,4aS,5S)-3-(2-Chlor-6-fluorbenzyliden)-4,4a,5,6,7,8-hexahydro-5-hydroxy-1,4a-dimethyl-naphthalen-2(3H)-one,
(3E,4aS,5S)-3-[(Benzofuran-2-yl)methylen]-4,4a,5,6,7,8-hexahydro-5-hydroxy-1,4a-dimethyl-naphthalen-2(3H)-one,
(3E,4aS,5S)-3-(3,4-bimethoxybenzyliden)-4,4a,5,6,7,8-hexahydro-5-hydroxy-1,4a-dimethyl-naphthalen-2(3H)-one,
(3E,4aS,5S)-3-(3,4,5-Trimethoxybenzyliden)-4,4a,5,6,7,8-hexahydro-5-hydroxy-1,4a-dimethylnaphthalen-2(3H)-one,
(3E,4aS,5S)-3-(4-Chlorobenzyliden)-4,4a,5,6,7,8-hexahydro-5-hydroxy-1,4a-dimethylnaphthalen-2(3H)-one,
(3E,4aS,5S)-3-[(Furan-3-yl)methylen]-4,4a,5,6,7,8-hexahydro-5-hydroxy-1,4a-dimethylnaphthalen-2(3H)-one,
(3E,4aS,5S)-3-[(4-Bromthiophen-2-yl)methylen]-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-[(pyridin-3-yl)methylen]naphthalen-2(3H)-one,
(3E,4aS,5S)-3-[4-(Methylthio)benzyliden]-4,4a,5,6,7,8-hexahydro-5-hydroxy-1,4a-dimethylnaphthalen-2(3H)-one,
(3E,4aS,5S)-3-[(4-Chlorthiophen-2-yl)methylen]-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-[(1-methyl-1H-pyrrol-2-yl)methylen]naphthalen-2(3H)-one,
(3E,4aS,5S)-3-(2-Hydroxy-4-methoxybenzyliden)-4,4a,5,6,7,8-hexahydro-5-hydroxy-1,4a-dimethylnaphthalen-2(3H)-one,
(3E,4aS,5S)-3-(2-Ethyl-4-methoxybenzyliden)-4,4a,5,6,7,8-hexahydro-5-hydroxy-1,4a-dimethylnaphthalen-2(3H)-one,
(3E,4aS,5S)-3-(4-Methoxy-2-methylbenzyliden)-4,4a,5,6,7,8-hexahydro-5-hydroxy-1,4a-dimethylnaphthalen-2(3H)-one,
(3E,4aS,5S)-3-(4-Nitrobenzyliden)-4,4a,5,6,7,8-hexahydro-5-hydroxy-1,4a-dimethylnaphthalen-2(3H)-one,
(3E,4aS,5S)-3-Benzyliden-4,4a,5,6,7,8-hexahydro-5-hydroxy-1,4a-dimethyl-naphthalen-2(3H)-one,
(3E,4aS,5S)-4,4a,5,6,7,8-Hexahydro-5-hydroxy-1,4a-dimethyl-3-[(4-methylthiophen-2-yl)methylen]naphthalen-2(3H)-one,
(3E,4aS,5S)-3-(2-Methyl-4-octyl-benzyliden)-4,4a,5,6,7,8-hexahydro-5-hydroxy-1,4a-dimethyl-naphthalen-2(3H)-one,
(3E,4aS,5S)-3-(3-Methylbenzyliden)-4,4a,5,6,7,8-hexahydro-5-hydroxy-1,4a-dimethylnaphthalen-2(3H)-one,
(3E,4aS,5S)-3-Dodecyliden-4,4a,5,6,7,8-hexahydro-5-hydroxy-1,4a-dimethylnaphthalen-2(3H)-one,
(3E,4aS,5S)-3-(4-Ethyl-2-methylbenzyliden)-4,4a,5,6,7,8-hexahydro-5-hydroxy-1,4a-dimethylnaphthalen-2(3H)-one,
(3E,4aS,5S)-3-(4-Methoxybenzyliden)-4,4a,5,6,7,8-hexahydro-5-hydroxy-1,4a-dimethylnaphthalen-2(3H)-one,
(2E,8aS)-2-[(Furan-2-yl)methylen]-3,4,6,7,8,8a-hexahydro-6-hydroxy-5,8a-dimethylnaphthalen-1(2H)-one,
3-{(1E)-[(4S,4aS)-1,2,3,4,4a,5-Hexahydro-4-hydroxy-4a,8-dimethyl-7-oxonaphthalen-6(7H)-yliden]methyl}phenylacetat,
(3E,4aS,5S)-3-[(5-Bromthiophen-2-yl)methylen]-4,4a,5,6,7,8-hexahydro-5-hydroxy-1,4a-dimethylnaphthalen-2(3H)-one,
(3E,4aS,5S)-3-(2,4-Difluorbenzyliden)-4,4a,5,6,7,8-hexahydro-5-hydroxy-1,4a-dimethylnaphthalen-2(3H)-one,
(3E,4aS,5S)-3-[(E)-But-2-enyliden]-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-[(thiophen-2-yl)methylen]naphthalen-2(3H)-one,
4-{(1E)-[(4S,4aS)-1,2,3,4,4a,5-Hexahydro-4-hydroxy-4a,8-dimethyl-7-oxonaphthalen-6(7H)-yliden]methyl}phenylacetat,
(3E,4aS,5S)-3-Butyliden-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimeth-yl-3-[(3-methylthiophen-2-yl)methylen]naphthalen-2(3H)-one,
(3E,4aS,5S)-3-[(Furan-2-yl)methylen]-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-[(pyridin-4-yl)methylen]naphthalen-2(3H)-one,
(3E,4aS,5S)-3-[4-(2-Phenylethinyl)benzyliden]-4,4a,5,6,7,8-hexahydro-5-hydroxy-1,4a-dimethylnaphthalen-2(3H)-one,
(3E,4aS,5S)-3-[4-(2-Methoxy-ethinyl)benzyliden]-4,4a,5,6,7,8-hexahydro-5-hydroxy-1,4a-dimethylnaphthalen-2(3H)-one,
(3E,4aS,5S)-3-[4-(3-tert-Butoxybut-1-inyl)benzyliden]-4,4a,5,6,7,8-hexahydro-5-hydroxy-1,4a-dimethylnaphthalen-2(3H)-one,
(3E,4aS,5S)-3-[4-(2-Cyclo-hexenylethinyl)benzyliden]-4,4a,5,6,7,8-hexahydro-5-hydroxy-1,4a-dimethylnaphthalen-2(3H)-one,
(3E,4aS,5S)-3-[4-(Hex-1-inyl)benzyliden]-4,4a,5,6,7,8-hexahydro-5-hydroxy-1,4a-dimethyl-naphthalen-2(3H)-one,
(3E,4aS,5S)-3-[4-(2-p-Tolylethinyl)benzyliden]-4,4a,5,6,7,8-hexahydro-5-hydroxy-1,4a-dimethyl-naphthalen-2(3H)-one,
(3E,4aS,5S)-3-[4-(Nona-1,8-diinyl)benzyliden]-4,4a,5,6,7,8-hexahydro-5-hydroxy-1,4a-dimethyl-naphthalen-2(3H)-one,
(3E,4aS,5S)-3-[4-(Hexadec-1-inyl)benzyliden]-4,4a,5,6,7,8-hexahydro-5-hydroxy-1,4a-dimethyl-naphthalen-2(3H)-one,
(3E,4aS,5S)-3-[4-(3-Hydroxy-3-methylbut-1-inyl)benzyliden]-4,4a,5,6,7,8-hexahydro-5-hydroxy-1,4a-dimethyl-naphthalen-2(3H)-one,
(3E,4aS,5S)-3-[4-(Dodec-1-inyl)benzyliden]-4,4a,5,6,7,8-hexahydro-5-hydroxy-1,4a-dimethyl-naphthalen-2(3H)-one,
(3E,4aS,5S)-3-[4-(3,3-Diethoxyprop-1-inyl)benzyliden]-4,4a,5,6,7,8-hexahydro-5-hydroxy-1,4a-dimethyl naphthalen-2(3H)-one,
6-{4-{(1E)-[(4S,4aS)-1,2,3,4,4a,5-Hexahydro-4-hydroxy-4a,8-dimethyl-7-oxonaphthalen-6(7H)-yliden]-methyl}phenyl}hex-5-in-nitril,
(3E,4aS,5S)-3-{4-[2-(1-Hydroxycyclohexyl)ethinyl]benzyliden}-4,4a,5,6,7,8-hexahydro-5-hydroxy-1,4a-dimethylnaphthalen-2(3H)-one,
(3E,4aS,5S)-3-{4-[2-(3-Fluorphenyl)ethinyl]benzyliden}-4,4a,5,6,7,8-hexahydro-5-hydroxy-1,4a-dimethylnaphthalen-2(3H)-one,
(3E,4aS,5S)-3-{4-[3-(N-Ethyl-N-benzylamino)prop-1-inyl]benzyliden}-4,4a,5,6,7,8-hexahydro-5-hydroxy-1,4a-dimethylnaphthalen-2(3H)-one,
(3E,4aS,5S)-3-{4-[3-(Dimethylamino)prop-1-inyl]benzyliden}-4,4a,5,6,7,8-hexahydro-5-hydroxy-1,4a-dimethylnaphthalen-2(3H)-one,
(3E,4aS,5S)-3-[4-(3-Phenylprop-1-inyl)benzyliden]-4,4a,5,6,7,8-hexahydro-5-hydroxy-1,4a-dimethyl naphthalen-2(3H)-one,
(3E,4aS,5S)-3-{4-[2-(Trimethylsilyl)ethinyl]benzyliden}-4,4a,5,6,7,8-hexahydro-5-hydroxy-1,4a-dimethylnaphthalen-2(3H)-one,
(3E,4aS,5S)-3-[4-(17-Ethinyl-ostradiol)benzyliden]-4,4a,5,6,7,8-hexahydro-5-hydroxy-1,4a-dimethylnaphthalen-2(3H)-one,
(3E,4aS,5S)-3-[4-(4-Hydroxybut-1-inyl)benzyliden]-4,4a,5,6,7,8-hexahydro-5-hydroxy-1,4a-dimethylnaphthalen-2(3H)-one,
(3E,4aS,5S)-3-[4-(3-Methylbut-3-en-1-inyl)benzyliden]-4,4a,5,6,7,8-hexahydro-5-hydroxy-1,4a-di methyl naphthalen-2(3H)-one,
(3E,4aS,5S)-3-[4-(3-Methyl but-1-inyl)benzyliden]-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-3-{2-[(1S,3R,4S,5R)-1,3,4,5-tetrahydroxycyclohexyl]-ethyliden}-1,4a-dimethyl naphthalen-2(3H)-one,
(3E,4aS,5S)-3-[3-(Furan-2-yl)propyliden]-4,4a,5,6,7,8-hexahydro-5-hydroxy-1,4a-dimethylnaphthalen-2(3H)-one,
(3E,4aS,5S)-3-[(E)-3-(Furan-2-yl)allyliden]-4,4a,5,6,7,8-hexahydro-5-hydroxy-1,4a-dimethylnaphthalen-2(3H)-one,
(3E,4aS,5S)-3-(2,3,4,5,6-Pentamethylbenzyliden)-4,4a,5,6,7,8-hexahydro-5-hydroxy-1,4a-dimethyl naphthalen-2(3H)-one,
(3E,4aS,5S)-3-[(Furan-3-yl)methylen]-4,4a,5,6,7,8-hexahydro-5-hydroxy-1,4a-dimethylnaphthalen-2(3H)-one,
(3E,4aS,5S)-3-(2-Brombenzyliden)-4,4a,5,6,7,8-hexahydro-5-hydroxy-1,4a-dimethylnaphthalen-2(3H)-one,
(3E,4aS,5S)-3-(3-Brombenzyliden)-4,4a,5,6,7,8-hexahydro-5-hydroxy-1,4a-dimethylnaphthalen-2(3H)-one,
(3E,4aS,5S)-3-(4-Brombenzyliden)-4,4a,5,6,7,8-hexahydro-5-hydroxy-1,4a-dimethylnaphthalen-2(3H)-one,
(3E,4aS,5S)-3-[4-(Benzyloxy)benzyliden]-4,4a,5,6,7,8-hexahydro-5-hydroxy-1,4a-dimethylnaphthalen-2(3H)-one,
(3E,4aS,5S)-3-(2-Iodbenzyliden)-4,4a,5,6,7,8-hexahydro-5-hydroxy-1,4a-dimethylnaphthalen-2(3H)-one,
(3E,4aS,5S)-3-(3-Iodbenzyliden)-4,4a,5,6,7,8-hexahydro-5-hydroxy-1,4a-dimethylnaphthalen-2(3H)-one,
(3E,4aS,5S)-3-(4-Iodbenzyliden)-4,4a,5,6,7,8-hexahydro-5-hydroxy-1,4a-dimethylnaphthalen-2(3H)-one,
(3E,4aS,5S)-3-(3-Brombutyliden)-4,4a,5,6,7,8-hexahydro-5-hydroxy-1,4a-dimethylnaphthalen-2(3H)-one,
(3E,4aS,5S)-3-[4-(3-Methylbut-3-en-1-inyl)benzyliden]-4,4a,5,6,7,8-hexahydro-5-hydroxy-1,4a-dimethyl naphthalen-2(3H)-one,
(3E,4aS,5S)-3-[4-(5-Phenylpent-1-inyl)benzyliden]-4,4a,5,6,7,8-hexahydro-5-hydroxy-1,4a-dimethylnaphthalen-2(3H)-one,
(3E,4aS,5S)-3-[4-(2-Cyclohexyl-ethinyl)benzyliden]-4,4a,5,6,7,8-hexahydro-5-hydroxy-1,4a-dimethylnaphthalen-2(3H)-one,
(E,4aS,5S)-3-{4-[2-(1-Hydroxycyclohexyl)ethinyl]-benzyliden}-octahydro-5-hydroxy-1,4a-dimethylnaphthalen-2(1H)-one,
(3E,4aS,5S)-3-[4-(Octadec-1-inyl)benzyliden]-4,4a,5,6,7,8-hexahydro-5-hydroxy-1,4a-dimethylnaphthalen-2(3H)-one,
(3E,4aS,5S)-3-{4-[2-(9-Hydroxy-9H-fluoren-9-yl)ethinyl]benzyliden}-4,4a,5,6,7,8-hexahydro-5-hydroxy-1,4a-dimethylnaphthalen-2(3H)-one,
(1S,8aS)-6-(4-Iodphenylamino)-1,2,3,4,6,7,8,8a-octahydro-5,8a-dimethylnaphthalen-1-ol,
(3E,4aS,5S)-3-[4-(2-Methoxy-4-nitrophenylamino)benzyliden]-4,4a,5,6,7,8-hexahydro-5-hydroxy-1,4a-dimethylnaphthalen-2(3H)-one,
(3E,4aS,5S)-3-[4-(4-Iodphenylamino)benzyliden]-4,4a,5,6,7,8-hexahydro-5-hydroxy-1,4a-dimethylnaphthalen-2(3H)-one,
(3E,4aS,5S)-3-[(Furan-3-yl)methylen]-4,4a,5,6,7,8-hexahydro-5-hydroxy-1,4a-dimethylnaphthalen-2(3H)-one,
(3E,4aS,5S)-3-(3-Brombenzyliden)-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-(2-methylbutyliden)-naphthalen-2(3H)-one,
(E,4aS,5S)-3-(4-Chlorbenzyliden)-octahydro-5-hydroxy-1,4a-dimethylnaphthalen-2(1H)-one,
(3E,4aS,5S)-3-(3-Fluor-4-methoxybenzyliden)-4,4a,5,6,7,8-hexahydro-5-hydroxy-1,4a-dimethylnaphthalen-2(3H)-one,
(3E,4aS,5S)-3-(4-Fluorbenzyliden)-4,4a,5,6,7,8-hexahydro-5-hydroxy-1,4a-dimethylnaphthalen-2(3H)-one,
(E,4aS,5S)-Octahydro-5-hydroxy-1,4a-dimethyl-3-[(1-methyl-1H-pyrrol-2-yl)methylen]naphthalen-2(1H)-one,
(E,4aS,5S)-3-(3,4-Difluorbenzyliden)-octahydro-5-hydroxy-1,4a-dimethylnaphthalen-2(1H)-one,
(E,4aS,5S)-3-(2-Chlor-6-fluorbenzyliden)-octahydro-5-hydroxy-1,4a-dimethylnaphthalen-2(1H)-one,
(E,4aS,5S)-3-(4-Ethyl-2-methylbenzyliden)-octahydro-5-hydroxy-1,4a-dimethylnaphthalen-2(1H)-one,
(3E,4aS,5S)-3-[4-(Undec-1-inyl)benzyliden]-4,4a,5,6,7,8-hexahydro-5-hydroxy-1,4a-dimethylnaphthalen-2(3H)-one,
(3E,4aS,5S)-3-[4-(Oct-1-inyl)benzyliden]-4,4a,5,6,7,8-hexahydro-5-hydroxy-1,4a-dimethylnaphthalen-2(3H)-one,
(3E,4aS,5S)-3-[4-(Tetradec-1-inyl)benzyliden]-4,4a,5,6,7,8-hexahydro-5-hydroxy-1,4a-dimethylnaphthalen-2(3H)-one,
(3E,4aS,5S)-3-{4-[2-(4-tert-Butylphenyl)ethinyl]benzyliden}-4,4a,5,6,7,8-hexahydro-5-hydroxy-1,4a-dimethyl naphthalen-2(3H)-one,
(3E,4aS,5S)-3-[4-(3,3-Dimethylbut-1-inyl)benzyliden]-4,4a,5,6,7,8-hexahydro-5-hydroxy-1,4a-dimethylnaphthalen-2(3H)-one,
(3E,4aS,5S)-3-{4-[2-(1-Hydroxycyclopentyl)ethinyl]-benzyliden}-4,4a,5,6,7,8-hexahydro-5-hydroxy-1,4a-dimethylnaphthalen-2(3H)-one,
(1S,8aS)-1,2,3,4,6,7,8,8a-Octahydro-5,8a-dimethyl-6-methylennaphthalen-1-ol,
(1S,7E,8aS)-7-Benzyliden-1,2,3,4,6,7,8,8a-octahydro-5,8a-dimethyl-6-methylennaphthalen-1-ol,
(1S,7E,8aS)-1,2,3,4,6,7,8,8a-Octahydro-5,8a-dimethyl-6-methylen-7-pentylidennaphthalen-1-ol,
(2E)-Ethyl 2-[(4S,4aS)-2,3,4,4a,5,6-hexahydro-4-hydroxy-4a,8-dimethylnaphthalen-7(1H)-yliden]acetat,
(1S,6E,8aS)-6-Hexyliden-1,2,3,4,6,7,8,8a-octahydro-5,8a-dimethylnaphthalen-1-ol,
(1S,6E,8aS)-6-Benzyliden-1,2,3,4,6,7,8,8a-octahydro-5,8a-dimethylnaphthalen-1-ol,
(1S,8aS)-5-(3,5-Dimethoxybenzyl)-decahydro-5,8a-dimethyl-6-methylennaphthalen-1-ol,
(E,4aS,5S)-3-(4-Hydroxy-3-methoxybenzyliden)-octahydro-5-hydroxy-1,4a-dimethylnaphthalen-2(1H)-one,
(3E,4aS,5S)-3-[4-(3-Hydroxyprop-1-inyl)benzyliden]-4,4a,5,6,7,8-hexahydro-5-hydroxy-1,4a-dimethylnaphthalen-2(3H)-one,
(3E,4aS,5S)-3-{4-[2-(4-Chlorphenyl)ethinyl]benzyliden}-4,4a,5,6,7,8-hexahydro-5-hydroxy-1,4a-dimethylnaphthalen-2(3H)-one,
(3E,4aS,5S)-3-[4-(3-Phenylprop-1-inyl)benzyliden]-4,4a,5,6,7,8-hexahydro-5-hydroxy-1,4a-dimethylnaphthalen-2(3H)-one,
(3E,4aS,5S)-3-(2,3,4-Trihydroxybenzyliden)-4,4a,5,6,7,8-hexahydro-5-hydroxy-1,4a-dimethylnaphthalen-2(3H)-one,
(E,4aS,5S)-3-(2,3,4-Trihydroxybenzyliden)-octahydro-5-hydroxy-1,4a-dimethylnaphthalen-2(1H)-one,
(3E,4aS,5S)-3-(2,3,4-Trihydroxybenzyliden)-4,4a,5,6,7,8-hexahydro-5-hydroxy-4a-methylnaphthalen-2(3H)-one,
(3E,4aS,5S)-3-(2,3,4-Trihydroxybenzyliden)-4a-benzyl-4,4a,5,6,7,8-hexahydro-5-hydroxy-1-methylnaphthalen-2(3H)-one,
(3E,4aS,5S)-4,4a,5,6,7,8-Hexahydro-5-hydroxy-3-[(R)-3-hydroxybutyliden]-1,4a-dimethylnaphthalen-2(3H)-one,
(E,4aS,5S)-Octahydro-5-hydroxy-3-[(R)-3-hydroxybutyliden]-1,4a-dimethylnaphthalen-2(1H)-one,
(3E,4aS,5S)-4,4a,5,6,7,8-Hexahydro-5-hydroxy-3-[(R)-3-hydroxybutyliden]-4a-methylnaphthalen-2(3H)-one,
(3E,4aS,5S)-4a-Benzyl-4,4a,5,6,7,8-hexahydro-5-hydroxy-3-[(R)-3-hydroxybutyliden]-1-methylnaphthalen-2(3H)-one,
(3E,4aS,5S)-3-[(1H-Indol-3-yl)methylen]-4,4a,5,6,7,8-hexahydro-5-hydroxy-1,4a-dimethylnaphthalen-2(3H)-one,
(E,4aS,5S)-3-[(1H-Indol-3-yl)methylen]-octahydro-5-hydroxy-1,4a-dimethylnaphthalen-2(1H)-one,
(3E,4aS,5S)-3-[(1H-Indol-3-yl)methylen]-4,4a,5,6,7,8-hexahydro-5-hydroxy-4a-methylnaphthalen-2(3H)-one,
(3E,4aS,5S)-3-[(1H-Indol-3-yl)methylen]-4a-benzyl-4,4a,5,6,7,8-hexahydro-5-hydroxy-1-methylnaphthalen-2(3H)-one,
(E,5S,8aS)-2-[(1H-Indol-3-yl)methylen]-octahydro-6-hydroxy-5,8a-dimethylnaphthalen-1(2H)-one,
(3E,4aS,5S)-3-[(E)-But-2-enyliden]-4,4a,5,6,7,8-hexahydro-5-hydroxy-1,4a-dimethylnaphthalen-2(3H)-one,
(3E,4aS,5S)-3-[(E)-But-2-enyliden]-octahydro-5-hydroxy-1,4a-dimethylnaphthalen-2(1H)-one,
(3E,4aS,5S)-3-[(E)-But-2-enyliden]-4,4a,5,6,7,8-hexahydro-5-hydroxy-4a-methylnaphthalen-2(3H)-one,
(3E,4aS,5S)-4a-Benzyl-3-[(E)-but-2-enyliden]-4,4a,5,6,7,8-hexahydro-5-hydroxy-1-methylnaphthalen-2(3H)-one,
(2E,8aS)-2-[(E)-But-2-enyliden]-octahydro-6-hydroxy-5,8a-dimethylnaphthalen-1(2H)-one,
(E,4aS,5S)-3-[4-(Oct-1-inyl)-benzyliden]-octahydro-5-hydroxy-1,4a-dimethylnaphthalen-2(1H)-one,
(3E,4aS,5S)-3-[4-(Oct-1-inyl)benzyliden]-4a-benzyl-4,4a,5,6,7,8-hexahydro-5-hydroxy-1-methylnaphthalen-2(3H)-one,
(E,4aS,5S)-3-[4-(Hexadec-1-inyl)benzyliden]-octahydro-5-hydroxy-1,4a-dimethylnaphthalen-2(1H)-one,
(3E,4aS,5S)-3-[4-(Hexadec-1-inyl)benzyliden]-4a-benzyl-4,4a,5,6,7,8-hexahydro-5-hydroxy-1-methylnaphthalen-2(3H)-one,
(E,4aS,5S)-3-[4-(Pent-1-inyl)benzyliden]-octahydro-5-hydroxy-1,4a-dimethylnaphthalen-2(1H)-one,
(3E,4aS,5S)-3-[4-(Pent-1-inyl)benzyliden]-4a-benzyl-4,4a,5,6,7,8-hexahydro-5-hydroxy-1-methylnaphthalen-2(3H)-one,
(E,4aS,5S)-3-[4-(Undec-1-inyl)benzyliden]-octahydro-5-hydroxy-1,4a-dimethylnaphthalen-2(1H)-one,
(3E,4aS,5S)-3-[4-(Undec-1-inyl)benzyliden]-4a-benzyl-4,4a,5,6,7,8-hexahydro-5-hydroxy-1-methylnaphthalen-2(3H)-one,
(E,4aS,5S)-3-[4-(Tetradec-1-inyl)benzyliden]-octahydro-5-hydroxy-1,4a-dimethylnaphthalen-2(1H)-one,
(3E,4aS,5S)-3-[4-(Tetradec-1-inyl)benzyliden]-4a-benzyl-4,4a,5,6,7,8-hexahydro-5-hydroxy-1-methylnaphthalen-2(3H)-one,
(3E,4aS,5S)-4a-Benzyl-4,4a,5,6,7,8-hexahydro-5-hydroxy-1-methyl-3-[(pyridin-4-yl)methylen]naphthalen-2(3H)-one,
(3E,4aS,5S)-3-[4-(2-Phenylethinyl)benzyliden]-4,4a,5,6,7,8-hexahydro-5-hydroxy-4a-methylnaphthalen-2(3H)-one,
(3E,4aS,5S)-3-{4-[2-(Trimethylsilyl)ethinyl]benzyliden}-4,4a,5,6,7,8-hexahydro-5-hydroxy-4a-methyl naphthalen-2(3H)-one,
(3E,4aS,5S)-3-{4-[2-(1-Hydroxycyclohexyl)ethinyl]benzyliden}-4,4a,5,6,7,8-hexahydro-5-hydroxy-4a-methylnaphthalen-2(3H)-one,
(1S,8aS)-6-(Butylamino)-1,2,3,4,6,7,8,8a-octahydro-5,8a-dimethylnaphthalen-1-ol,
(1S,8aS)-6-(Cyclohexylamino)-1,2,3,4,6,7,8,8a-octahydro-5,8a-dimethylnaphthalen-1-ol,
(1S,7E,8aS)-7-(4-Iodbenzyliden)-6-(cyclohexylamino)-1,2,3,4,6,7,8,8a-octahydro-5,8a-dimethylnaphthalen-1-ol
(1S,8aS)-6-(4-Iodophenylamino)-1,2,3,4,6,7,8,8a-octahydro-5,8a-dimethylnaphthalen-1-ol,
(1S,8aS)-6-(4-Iodophenylamino)-5-benzyl-decahydro-5,8a-dimethylnaphthalen-1-ol,
(1S,8aS)-6-(2-Methoxy-4-nitrophenylamino)-1,2,3,4,6,7,8,8a-octahydro-5,8a-dimethylnaphthalen-1-ol,
(1S,8aS)-5-(3,5-Dimethoxybenzyl)-6-(4-Iodophenylamino)-decahydro-5,8a-dimethylnaphthalen-1-ol,
(1S,8aS)-5-(3,5-Dimethoxybenzyl)-6-(cyclohexylamino)-decahydro-5,8a-dimethylnaphthalen-1-ol,
(1S,8aS)-5-(3,5-Dimethoxybenzyl)-6-(butylamino)-decahydro-5,8a-dimethylnaphthalen-1-ol,
(1S,8aS)-7-(4-Iodbenzyl)-6-(butylamino)-1,2,3,4,6,7,8,8a-octahydro-5,8a-dimethylnaphthalen-1-ol,
(4aS,5S)-3-[4-(3-tert-Butoxybut-1-inyl)benzyl]-4,4a,5,6,7,8-hexahydro-5-hydroxy-1,4a-dimethylnaphthalen-2(3H)-one,
(4aS,5S)-3-{4-[3-(N-Benzyl-N-methylamino)prop-1-inyl]benzyl}-4,4a,5,6,7,8-hexahydro-5-hydroxy-1,4a-dimethylnaphthalen-2(3H)-one,
(4aS,5S)-{3-[4-(2-(Pyridin-2-yl)ethinyl]benzyl}-4,4a,5,6,7,8-hexahydro-5-hydroxy-1,4a-dimethylnaphthalen-2(3H)-one,
(4aS,5S)-3-[4-(4-Hydroxybut-1-inyl)benzyl]-4,4a,5,6,7,8-hexahydro-5-hydroxy-1,4a-dimethylnaphthalen-2(3H)-one,
Ethyl-2-[(4aS,5S)-decahydro-5-hydroxy-1,4a-dimethyl-2-oxonaphthalen-1-yl]acetat,
(3E,4aS,5S)-3-[(9-Ethyl-9H-carbazol-3-yl)methylen]-4,4a,5,6,7,8-hexahydro-5-hydroxy-1,4a-dimethylnaphthalen-2(3H)-one,
(E,4aS,5S)-1-(3,5-Dimethoxy-benzyl)-3-(4-fluorbenzyliden)-octahydro-5-hydroxy-1,4a-dimethylnaphthalen-2(1H)-one,
(E,4aS,5S)-3-(3,4-Dimethoxybenzyliden)-1-(3,5-dimethoxy-benzyl)-octahydro-5-hydroxy-1,4a-dimethylnaphthalen-2(1H)-one,
(E,4aS,5S)-3-[(Benzofuran-2-yl)methylen]-octahydro-5-hydroxy-1,4a-dimethylnaphthalen-2(1H)-one,
(3E,4aS,5S)-Octahydro-3-[(4,5-dihydro-3-methyl-1H-pyrrol-2-yl)methylen]-5-hydroxy-1,4a-dimethylnaphthalen-2(1H)-one,
(3E,4aS,5S)-3-(2,4,5-Trimethoxybenzyliden)-4,4a,5,6,7,8-hexahydro-5-hydroxy-1,4a-dimethylnaphthalen-2(3H)-one,
(3E,4aS,5R)-4,4a,5,6,7,8-Hexahydro-5-hydroxy-1,4a-dimethyl-3-(perfluorbenzyliden)naphthalen-2(3H)-one,
(E,4aS,5S)-3-(4-Brombenzyliden)-octahydro-5-hydroxy-1,4a-dimethylnaphthalen-2(1H)-one,
(E,4aS,5S)-Octahydro-5-hydroxy-1,4a-dimethyl-3-[(1-methyl-1H-pyrrol-2-yl)methylen]naphthalen-2(1H)-one,
(E,4aS,5S)-Octahydro-5-hydroxy-1,4a-dimethyl-3-(2-methyl-butyliden)naphthalen-2(1H)-one,
(E,4aS,5S)-3-(4-Fluorbenzyliden)-octahydro-5-hydroxy-1,4a-dimethylnaphthalen-2(1H)-one,
(3E,4R,4aS,5S)-3-(4-Iodbenzyliden)-4,4a,5,6,7,8-hexahydro-5-hydroxy-4,4a-dimethylnaphthalen-2(3H)-one,
(3E,4R,4aS,5S)-3-(3-Chlorbenzyliden)-4,4a,5,6,7,8-hexahydro-5-hydroxy-4,4a-dimethylnaphthalen-2(3H)-one,
5-Hydroxy-1-methyl-7-phenyl-4,4a,5,6,7,8,-hexahydro-3H-naphthalen-2-one,
(3E,4aS,5S)-3-(3-Brombenzyliden)-4,4a,5,6,7,8-hexahydro-5-hydroxy-1,4a-dimethylnaphthalen-2(3H)-one,
(3E,4aS,5S)-3-(2-Brombenzyliden)-4,4a,5,6,7,8-hexahydro-5-hydroxy-1,4a-dimethylnaphthalen-2(3H)-one,
(3E,4aS,5S)-3-Ethyliden-4,4a,5,6,7,8-hexahydro-5-hydroxy-1,4a-dimethylnaphthalen-2(3H)-one,
(E,4aS,5S)-3-(2,3,4-Trimethoxy-benzyliden)-octahydro-5-hydroxy-1,4a-dimethylnaphthalen-2(1H)-one,
(E,4aS,5S)-3-[4-(Methylthio)-benzyliden]-octahydro-5-hydroxy-1,4a-dimethylnaphthalen-2(1H)-one,
(3E,4aS,5S)-Octahydro-5-hydroxy-1,4a-dimethyl-3-{[(S)-4-(prop-1-en-2-yl)cyclohex-1-enyl]-methylen}naphthalen-2(1H)-one,
(E,4aS,5S)-Octahydro-5-hydroxy-1,4a-dimethyl-3-[(5-methylfuran-2-yl)methylen]naphthalen-2(1H)-one,
(3E,4R,4aS,5S)-3-[2-(Benzyloxy)-benzyliden]-4,4a,5,6,7,8-hexahydro-5-hydroxy-4,4a-dimethylnaphthalen-2(3H)-one,
(3E,4R,4aS,5S)-3-[(Furan-3-yl)methylen]-4,4a,5,6,7,8-hexahydro-5-hydroxy-4,4a-dimethylnaphthalen-2(3H)-one,
(3E,4aS,5S)-3-(4-Brombenzyliden)-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-(2-methyl-butylidene)naphthalen-2(3H)-one,
(3E,4aS,5S)-3-(4-Fluorbenzyliden)-4,4a,5,6,7,8-hexahydro-5-hydroxy-1,4a-dimethylnaphthalen-2(3H)-one,
(E,4aS,5S)-3-(4-Iodbenzyliden)-octahydro-5-hydroxy-1,4a-dimethylnaphthalen-2(1H)-one,
(E,4aS,5S)-Octahydro-5-hydroxy-1,4a-dimethyl-3-(2-methylbutyliden)naphthalen-2(1H)-one,
(E,4aS,5S)-3-(2-Iodbenzyliden)-octahydro-5-hydroxy-1,4a-dimethylnaphthalen-2(1H)-one,
(E,4aS,5S)-3-(3-Chlorbenzyliden)-octahydro-5-hydroxy-1,4a-dimethylnaphthalen-2(1H)-one,
(3E,4R,4aS,5S)-3-(2-Fluorbenzyliden)-4,4a,5,6,7,8-hexahydro-5-hydroxy-4,4a-dimethylnaphthalen-2(3H)-one,
(3E,4aS,5S)-3-[(Furan-2-yl)methylen]-4,4a,5,6,7,8-hexahydro-5-hydroxy-4a-methylnaphthalen-2(3H)-one,
(3E,4aS,5S)-3-(2,3,4-Trimethoxybenzyliden)-4,4a,5,6,7,8-hexahydro-5-hydroxy-1,4a-dimethylnaphthalen-2(3H)-one,
(3E,4aS,5S)-3-[4-(Methylthio)benzyliden]-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-{[(S)-4-(prop-1-en-2-yl)cyclohex-1-enyl]methylen}-naphthalen-2(3H)-one,
(3E,4aS,5S)-4,4a,5,6,7,8-Hexahydro-5-hydroxy-1,4a-dimethyl-3-[(5-methylfuran-2-yl)methylen]naphthalen-2(3H)-one,
(E,4aS,5S)-3-[2-(Benzyloxy)-benzyliden]-octahydro-5-hydroxy-1,4a-dimethylnaphthalen-2(1H)-one,
(3E,4R,4aS,5S)-3-(3-Iodbenzyliden)-4,4a,5,6,7,8-hexahydro-5-hydroxy-4,4a-dimethylnaphthalen-2(3H)-one,
(3E,4aS,5S)-3-(4-Iodbenzyliden)-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-(2-methyl-butyliden)naphthalen-2(3H)-one,
(3E,4aS,5S)-3-(2-Iodbenzyliden)-4,4a,5,6,7,8-hexahydro-5-hydroxy-1,4a-dimethylnaphthalen-2(3H)-one,
(3E,4aS,5S)-3-(3-Chlorbenzyliden)-4,4a,5,6,7,8-hexahydro-5-hydroxy-1,4a-dimethylnaphthalen-2(3H)-one,
(E,4aS,5S)-3-(2-Fluorbenzyliden)-octahydro-5-hydroxy-1,4a-dimethylnaphthalen-2(1H)-one,
(3E,4R,4aS,5S)-3-(2,4,5-Trimethoxybenzyliden)-4,4a,5,6,7,8-hexahydro-5-hydroxy-4,4a-dimethylnaphthalen-2(3H)-one,
(3E,4aS,5S)-3-(4-Brombenzyliden)-4,4a,5,6,7,8-hexahydro-5-hydroxy-4a-methylnaphthalen-2(3H)-one,
(3E,4aS,5S)-3-[2-(Benzyloxy)-benzyliden]-4,4a,5,6,7,8-hexahydro-5-hydroxy-1,4a-dimethyl naphthalen-2(3H)-one,
(3E,4aS,5S)-4,4a,5,6,7,8-Hexahydro-5-hydroxy-1,4a-dimethyl-3-[(2,6,6-trimethylcyclohexa-1,3-dienyl)-methylen]naphthalen-2(3H)-one,
(E,4aS,5S)-3-(2,3,4,5,6-Pentamethylbenzyliden)-octahydro-5-hydroxy-1,4a-dimethylnaphthalen-2(1H)-one,
(E,4aS,5S)-3-(3-Iodbenzyliden)-octahydro-5-hydroxy-1,4a-dimethylnaphthalen-2(1H)-one,
(3E,4R,4aS,5S)-3-Benzyliden-4,4a,5,6,7,8-hexahydro-5-hydroxy-4,4a-dimethylnaphthalen-2(3H)-one,
(3E,4R,4aS,5S)-3-(3-Brombenzyliden)-4,4a,5,6,7,8-hexahydro-5-hydroxy-4,4a-dimethylnaphthalen-2(3H)-one,
(3E,4R,4aS,5S)-3-(2-Brombenzyliden)-4,4a,5,6,7,8-hexahydro-5-hydroxy-4,4a-dimethylnaphthalen-2(3H)-one,
(3E,4R,4aS,5S)-3-Ethyliden-4,4a,5,6,7,8-hexahydro-5-hydroxy-4,4a-dimethylnaphthalen-2(3H)-one,
(E,4aS,5S)-3-(2,3,4-Trimethoxy-benzyliden)-octahydro-5-hydroxy-1,4a-dimethylnaphthalen-2(1H)-one,
(3E,4aS,5S)-3-(2-Fluorbenzyliden)-4,4a,5,6,7,8-hexahydro-5-hydroxy-1,4a-dimethylnaphthalen-2(3H)-one,
(3E,4aS,5S)-4,4a,5,6,7,8-Hexahydro-5-hydroxy-1,4a-dimethyl-3-(2-methylpentyliden)-naphthalen-2(3H)-one,
(E,4aS,5S)-3-(2,4,5-Trimethoxybenzyliden)-octahydro-5-hydroxy-1,4a-dimethylnaphthalen-2(1H)-one,
(E,4aS,5S)-Octahydro-5-hydroxy-1,4a-dimethyl-3-(perfluorbenzyliden)naphthalen-2(1H)-one,
(3E,4R,4aS,5S)-3-(4-Brombenzyliden)-4,4a,5,6,7,8-hexahydro-5-hydroxy-4,4a-dimethylnaphthalen-2(3H)-one,
(3E,4R,4aS,5S)-3-(4-Fluorbenzyliden)-4,4a,5,6,7,8-hexahydro-5-hydroxy-4,4a-dimethylnaphthalen-2(3H)-one,
(3E,4aS,5S)-3-(4-Iodbenzyliden)-4,4a,5,6,7,8-hexahydro-5-hydroxy-4a-methylnaphthalen-2(3H)-one, (3E,4aS,5S)-3-(2,3,4,5,6-Pentamethylbenzyliden)-4,4a,5,6,7,8-hexahydro-5-hydroxy-1,4a-dimethyl naphthalen-2(3H)-one,
(3E,4aS,5S)-3-(3-Iodbenzyliden)-4,4a,5,6,7,8-hexahydro-5-hydroxy-1,4a-dimethylnaphthalen-2(3H)-one,
(3E,4aS,5S)-3-(3-Fluorbenzyliden)-4,4a,5,6,7,8-hexahydro-5-hydroxy-1,4a-dimethylnaphthalen-2(3H)-one,
(E,4aS,5S)-3-(3-Brombenzyliden)-octahydro-5-hydroxy-1,4a-dimethylnaphthalen-2(1H)-one,
(E,4aS,5S)-3-(2-Brombenzyliden)-octahydro-5-hydroxy-1,4a-dimethylnaphthalen-2(1H)-one,
(E,4aS,5S)-3-Ethyliden-octahydro-5-hydroxy-1,4a-dimethylnaphthalen-2(1H)-one,
(3E,4R,4aS,5S)-3-(2,3,4-Trimethoxybenzyliden)-4,4a,5,6,7,8-hexahydro-5-hydroxy-4,4a-dimethylnaphthalen-2(3H)-one,
(3E,4R,4aS,5S)-3-[4-(Methylthio)benzyliden]-4,4a,5,6,7,8-hexahydro-5-hydroxy-4,4a-dimethylnaphthalen-2(3H)-one,
(3E,4aS,5S)-3-[2-(Benzyloxy)benzyliden]-4,4a,5,6,7,8-hexahydro-5-hydroxy-4a-methylnaphthalen-2(3H)-one,
(3E,4aS,5S)-3-(2-Fluorbenzyliden)-4,4a,5,6,7,8-hexahydro-5-hydroxy-4a-methylnaphthalen-2(3H)-one,
(3E,4aS,5S)-4,4a,5,6,7,8-Hexahydro-5-hydroxy-4a-methyl-3-(2-methylpentyliden)naphthalen-2(3H)-one,
4-{(1E)-[(4S,4aS)-1,2,3,4,4a,5-Hexahydro-4-hydroxy-4a-methyl-7-oxonaphthalen-6(7H)-yliden]methyl}benzoic acid,
(3E,4aS,5S)-4a-Benzyl-3-[(furan-2-yl)methylen]-4,4a,5,6,7,8-hexahydro-5-hydroxy-1-methylnaphthalen-2(3H)-one,
(3E,4aS,5S)-3-(2,4,5-Trimethoxy-benzyliden)-4a-benzyl-4,4a,5,6,7,8-hexahydro-5-hydroxy-1-methylnaphthalen-2(3H)-one,
(3E,4aS,5S)-4a-Benzyl-4,4a,5,6,7,8-hexahydro-5-hydroxy-1-methyl-3-(perfluorbenzyliden)naphthalen-2(3H)-one,
(3E,4R,4aS,5S)-3-(4-Brombenzyliden)-4a-allyl-4,4a,5,6,7,8-hexahydro-5-hydroxy-4-methylnaphthalen-2(3H)-one,
(3E,4R,4aS,5S)-4a-Allyl-4,4a,5,6,7,8-hexahydro-5-hydroxy-4-methyl-3-[(1-methyl-1H-pyrrol-2-yl)methylen]naphthalen-2(3H)-one,
(3E,4R,4aS,5S)-4a-Allyl-4,4a,5,6,7,8-hexahydro-5-hydroxy-4-methyl-3-(2-methylbutylidene)-naphthalen-2(3H)-one,
(E, 5S)-2-(4-Iodbenzyliden)-octahydro-6-hydroxy-5-methylnaphthalen-1(2H)-one,
(3E,4aS,5S)-3-(3-Iodbenzyliden)-4,4a,5,6,7,8-hexahydro-5-hydroxy-4a-methylnaphthalen-2(3H)-one,
(3E,4aS,5S)-3-(3-Fluorbenzyliden)-4,4a,5,6,7,8-hexahydro-5-hydroxy-4a-methylnaphthalen-2(3H)-one,
(3E,4aS,5S)-4a-Benzyl-3-benzyliden-4,4a,5,6,7,8-hexahydro-5-hydroxy-1-methylnaphthalen-2(3H)-one,
(3E,4aS,5S)-3-(3-Brombenzyliden)-4a-benzyl-4,4a,5,6,7,8-hexahydro-5-hydroxy-1-methylnaphthalen-2(3H)-one,
(3E,4aS,5S)-4a-Benzyl-3-ethyliden-4,4a,5,6,7,8-hexahydro-5-hydroxy-1-methylnaphthalen-2(3H)-one,
(3E,4R,4aS,5S)-3-(2,3,4-Trimethoxybenzyliden)-4a-allyl-4,4a,5,6,7,8-hexahydro-5-hydroxy-4-methylnaphthalen-2(3H)-one,
(3E,4R,4aS,5S)-3-[4-(Methylthio)-benzyliden]-4a-allyl-4,4a,5,6,7,8-hexahydro-5-hydroxy-4-methylnaphthalen-2(3H)-one,
(3E,4R,4aS,5S)-4a-Allyl-4,4a,5,6,7,8-hexahydro-5-hydroxy-4-methyl-3-{[(S)-4-(prop-1-en-2-yl)cyclohex-1-enyl]methylen}naphthalen-2(3H)-one,
(3E,4R,4aS,5S)-4a-Allyl-4,4a,5,6,7,8-hexahydro-5-hydroxy-4-methyl-3-[(5-methylfuran-2-yl)methylen]naphthalen-2(3H)-one,
(E,5S,8aS)-2-(2-(Benzyloxy)-benzyliden)-octahydro-6-hydroxy-5,8a-dimethylnaphthalen-1(2H)-one,
(3E,4aS,5S)-3-(2,4,5-Trimethoxybenzyliden)-4,4a,5,6,7,8-hexahydro-5-hydroxy-4a-methylnaphthalen-2(3H)-one,
(3E,4aS,5S)-4,4a,5,6,7,8-Hexahydro-5-hydroxy-4a-methyl-3-(perfluorbenzyliden)naphthalen-2(3H)-one,
(3E,4aS,5S)-4a-Benzyl-4,4a,5,6,7,8-hexahydro-5-hydroxy-1-methyl-3-(2-methylbutyliden)naphthalen-2(3H)-one,
(3E,4aS,5S)-3-(4-Fluorbenzyliden)-4a-benzyl-4,4a,5,6,7,8-hexahydro-5-hydroxy-1-methylnaphthalen-2(3H)-one,
(3E,4R,4aS,5S)-3-(4-Iodbenzyliden)-4a-allyl-4,4a,5,6,7,8-hexahydro-5-hydroxy-4-methylnaphthalen-2(3H)-one,
(3E,4R,4aS,5S)-4a-Allyl-4,4a,5,6,7,8-hexahydro-5-hydroxy-4-methyl-3-(2-methylbutyliden)-naphthalen-2(3H)-one,
(3E,4R,4aS,5S)-3-(2-Iodbenzyliden)-4a-allyl-4,4a,5,6,7,8-hexahydro-5-hydroxy-4-methylnaphthalen-2(3H)-one,
(E,5S,8aS)-2-(2-Fluorbenzyliden)-octahydro-6-hydroxy-5,8a-dimethylnaphthalen-1(2H)-one,
(3E,4aS,5S)-3-(3-Brombenzyliden)-4,4a,5,6,7,8-hexahydro-5-hydroxy-4a-methylnaphthalen-2(3H)-one,
(3E,4aS,5S)-3-Ethyliden-4,4a,5,6,7,8-hexahydro-5-hydroxy-4a-methylnaphthalen-2(3H)-one,
(3E,4aS,5S)-3-(2,3,4-Trimethoxybenzyliden)-4a-benzyl-4,4a,5,6,7,8-hexahydro-5-hydroxy-1-methylnaphthalen-2(3H)-one,
(3E,4R,4aS,5S)-3-[2-(Benzyloxy)-benzyliden]-4a-allyl-4,4a,5,6,7,8-hexahydro-5-hydroxy-4-methylnaphthalen-2(3H)-one,
(3E,4R,4aS,5S)-4a-Allyl-4,4a,5,6,7,8-hexahydro-5-hydroxy-4-methyl-3-{[(R)-4-(prop-1-en-2-yl)cyclohex-1-enyl]-methylen}naphthalen-2(3H)-one,
(3E,4R,4aS,5S)-4a-Allyl-3-[(furan-3-yl)methylen]-4,4a,5,6,7,8-hexahydro-5-hydroxy-4-methylnaphthalen-2(3H)-one,
(3E,4R,4aS,5S)-4a-Allyl-4,4a,5,6,7,8-hexahydro-5-hydroxy-4-methyl-3-[(2,6,6-trimethyl-cyclohexa-1,3-dienyl)methylen]-naphthalen-2(3H)-one,
(E, 5S,8aS)-2-(2,3,4,5,6-Pentamethyl-benzyliden)-octahydro-6-hydroxy-5,8a-dimethylnaphthalen-1(2H)-one,
(3E,4aS,5S)-4,4a,5,6,7,8-Hexahydro-5-hydroxy-4a-methyl-3-[(1-methyl-1H-pyrrol-2-yl)methylen]naphthalen-2(3H)-one,
(3E,4aS,5S)-4,4a,5,6,7,8-Hexahydro-5-hydroxy-4a-methyl-3-(2-methylbutyliden)naphthalen-2(3H)-one,
(3E,4aS,5S)-3-(4-Fluorbenzyliden)-4,4a,5,6,7,8-hexahydro-5-hydroxy-4a-methylnaphthalen-2(3H)-one,
(3E,4aS,5S)-4a-Benzyl-4,4a,5,6,7,8-hexahydro-5-hydroxy-1-methyl-3-(2-methylbutyliden)naphthalen-2(3H)-one,
(3E,4R,4aS,5S)-3-(2-Fluorbenzyliden)-4a-allyl-4,4a,5,6,7,8-hexahydro-5-hydroxy-4-methylnaphthalen-2(3H)-one,
(3E,4R,4aS,5S)-4a-Allyl-4,4a,5,6,7,8-hexahydro-5-hydroxy-4-methyl-3-(2-methylpentyliden)-naphthalen-2(3H)-one,
(E, 5S,8aS)-2-[(Furan-2-yl)methylen]-octahydro-6-hydroxy-5,8a-dimethylnaphthalen-1(2H)-one,
(E, 5S,8aS)-2-(2,4,5-Trimethoxy-benzyliden)-octahydro-6-hydroxy-5,8a-dimethylnaphthalen-1(2H)-one,
(3E,4aS,5S)-3-[4-(Methylthio)-benzyliden]-4,4a,5,6,7,8-hexahydro-5-hydroxy-4a-methylnaphthalen-2(3H)-one,
(3E,4aS,5S)-4,4a,5,6,7,8-Hexahydro-5-hydroxy-4a-methyl-3-{[(S)-4-(prop-1-en-2-yl)cyclohex-1-enyl]methylen}naphthalen-2(3H)-one,
(3E,4aS,5S)-4,4a,5,6,7,8-Hexahydro-5-hydroxy-4a-methyl-3-[(5-methylfuran-2-yl)methylen]naphthalen-2(3H)-one,
(3E,4aS,5S)-4,4a,5,6,7,8-Hexahydro-5-hydroxy-4a-methyl-3-[(5-methylfuran-2-yl)methylen]naphthalen-2(3H)-one,
(3E,4aS,5S)-4a-Benzyl-4,4a,5,6,7,8-hexahydro-5-hydroxy-1-methyl-3-{[(S)-4-(prop-1-en-2-yl)cyclohex-1-enyl]methylen}naphthalen-2(3H)-one,
(3E,4R,4aS,5S)-3-(2,3,4,5,6-Pentamethylbenzyliden)-4a-allyl-4,4a,5,6,7,8-hexahydro-5-hydroxy-4-methylnaphthalen-2(3H)-one,
(3E,4R,4aS,5S)-3-(3-Iodbenzyliden)-4a-allyl-4,4a,5,6,7,8-hexahydro-5-hydroxy-4-methylnaphthalen-2(3H)-one,
(3E,4R,4aS,5S)-3-(3-Fluorbenzyliden)-4a-allyl-4,4a,5,6,7,8-hexahydro-5-hydroxy-4-methylnaphthalen-2(3H)-one,
(E, 5S,8aS)-2-(3-Brombenzyliden)-octahydro-6-hydroxy-5,8a-dimethylnaphthalen-1(2H)-one,
(3E,4aS,5S)-4,4a,5,6,7,8-Hexahydro-5-hydroxy-4a-methyl-3-(2-methylbutyliden)naphthalen-2(3H)-one,
(3E,4aS,5S)-3-(2-Iodbenzyliden)-4,4a,5,6,7,8-hexahydro-5-hydroxy-4a-methylnaphthalen-2(3H)-one,
(3E,4aS,5S)-3-(2-Fluorbenzyliden)-4a-benzyl-4,4a,5,6,7,8-hexahydro-5-hydroxy-1-methylnaphthalen-2(3H)-one,
(3E,4aS,5S)-4a-Benzyl-4,4a,5,6,7,8-hexahydro-5-hydroxy-1-methyl-3-(2-methylpentyliden)naphthalen-2(3H)-one,
(3E,4R,4aS,5S)-3-(2,4,5-Trimethoxybenzyliden)-4a-allyl-4,4a,5,6,7,8-hexahydro-5-hydroxy-4-methylnaphthalen-2(3H)-one,
(3E,4R,4aS,5S)-4a-Allyl-4,4a,5,6,7,8-hexahydro-5-hydroxy-4-methyl-3-propylidennaphthalen-2(3H)-one,
(3E,4R,4aS,5S)-4a-Allyl-4,4a,5,6,7,8-hexahydro-5-hydroxy-4-methyl-3-(perfluorbenzyliden)-naphthalen-2(3H)-one,
(E, 5S,8aS)-2-(4-Brombenzyliden)-octahydro-6-hydroxy-5,8a-dimethylnaphthalen-1(2H)-one,
(E, 5S,8aS)-Octahydro-6-hydroxy-5,8a-dimethyl-2-[(1-methyl-1H-pyrrol-2-yl)methylen]naphthalen-1(2H)-one,
(3E,4aS,5S)-4,4a,5,6,7,8-Hexahydro-5-hydroxy-4a-methyl-3-{[(R)-4-(prop-1-en-2-yl)cyclohex-1-enyl]methylen}naphthalen-2(3H)-one,
(3E,4aS,5S)-3-[(Furan-3-yl)methylen]-4,4a,5,6,7,8-hexahydro-5-hydroxy-4a-methylnaphthalen-2(3H)-one,
(3E,4aS,5S)-3-(3-Fluorbenzyliden)-4a-benzyl-4,4a,5,6,7,8-hexahydro-5-hydroxy-1-methylnaphthalen-2(3H)-one,
(3E,4R,4aS,5S)-4a-Allyl-3-benzyliden-4,4a,5,6,7,8-hexahydro-5-hydroxy-4-methylnaphthalen-2(3H)-one,
(E,5S,8aS)-2-(2,3,4-Trimethoxy-benzyliden)-octahydro-6-hydroxy-5,8a-dimethylnaphthalen-1(2H)-one,
(E,5S,8aS)-2-(4-(Methylthio)-benzyliden)-octahydro-6-hydroxy-5,8a-dimethylnaphthalen-1(2H)-one,
(E,5S,8aS)-Octahydro-6-hydroxy-5,8a-dimethyl-2-(2-methylbutyliden)naphthalen-1(2H)-one,
(E,5S,8aS)-2-(2-Iodbenzyliden)-octahydro-6-hydroxy-5,8a-dimethylnaphthalen-1(2H)-one,
(E,5S,8aS)-2-(3-Chlorbenzyliden)-octahydro-6-hydroxy-5,8a-dimethylnaphthalen-1(2H)-one,
(2E,5S,8aS)-Octahydro-6-hydroxy-5,8a-dimethyl-2-{[(S)-4-(prop-1-en-2-yl)cyclohex-1-enyl]methylen}naphthalen-1(2H)-one,
(E, 5S,8aS)-2-[(Furan-2-yl)methylen]-octahydro-6-hydroxy-5,8a-dimethylnaphthalen-1(2H)-one,
(2E,5S,8aS)-Octahydro-6-hydroxy-5,8a-dimethyl-2-[(2,6,6-trimethylcyclohexa-1,3-dienyl)-methylen]naphthalen-1(2H)-one,
(E, 5S,8aS)-Octahydro-6-hydroxy-5,8a-dimethyl-2-(2-methyl-pentyliden)naphthalen-1(2H)-one,
4-{(E)-[(1S,4aS)-Octahydro-2-hydroxy-1,4a-dimethyl-5-oxonaphthalen-6(1H)-yliden]methyl}benzoic acid,
(E,5S,8aS)-2-(3-Iodbenzyliden)-octahydro-6-hydroxy-5,8a-dimethylnaphthalen-1(2H)-one,
(E,5S,8aS)-2-(3-Fluorbenzyliden)-octahydro-6-hydroxy-5,8a-dimethylnaphthalen-1(2H)-one,
(E, 5S,8aS)-Octahydro-6-hydroxy-5,8a-dimethyl-2-propyliden-naphthalen-1(2H)-one,
(E, 5S,8aS)-Octahydro-6-hydroxy-5,8a-dimethyl-2-(perfluorbenzyliden)naphthalen-1(2H)-one,
(E, 5S,8aS)-2-(2-Brombenzyliden)-octahydro-6-hydroxy-5,8a-dimethylnaphthalen-1(2H)-one,
(E,5S,8aS)-2-Ethyliden-octahydro-6-hydroxy-5,8a-dimethylnaphthalen-1(2H)-one,
(E, 5S,8aS)-Octahydro-6-hydroxy-5,8a-dimethyl-2-(2-methylbutyliden)naphthalen-1(2H)-one,
(E, 5S,8aS)-2-(4-Fluorbenzyliden)-octahydro-6-hydroxy-5,8a-dimethylnaphthalen-1(2H)-one,
(2E,5S,8aS)-Octahydro-6-hydroxy-5,8a-dimethyl-2-{[(R)-4-(prop-1-en-2-yl)cyclohex-1-enyl]-methylen}naphthalen-1(2H)-one,
(E, 5S,8aS)-Octahydro-6-hydroxy-5,8a-dimethyl-2-[(5-methylfuran-2-yl)methylen]naphthalen-1(2H)-one,
(E, 11S,8aS)-7-[2-(2-Cyclohexylethinyl)benzyliden]-6-(butylamino)-decahydro-5,8a-dimethylnaphthalen-1-ol,
(E,4aS)-6-[2-(Hexadec-1-inyl)benzyliden]-5-(butylamino)-decahydro-1,4a-dimethylnaphthalen-2-ol,
(1S,7E,8aS)-7-{2-[2-(Trimethylsilyl)ethinyl]benzyliden}-6-(butylamino)-1,213,4,6,7,8,8a-octahydro-5,8a-dimethylnaphthalen-1-ol,
(E,1S,8aS)-7-{2-[2-(3-Fluorphenyl)ethinyl]benzyliden}-6-(butylamino)-decahydro-5,8a-dimethylnaphthalen-1-ol,
(1S,7E,8aS)-7-[2-(3-Methoxyprop-1-inyl)benzyliden]-6-(butyl-amino)-1,2,3,4,6,7,8,8a-octahydro-8a-methylnaphthalen-1-ol,
(1S,7E,8aS)-7-{2-[2-(Trimethylsilyl)ethyl]-benzyliden}-6-(butylamino)-1,2,3,4,6,7,8,8a-octahydro-5,8a-dimethylnaphthalen-1-ol,
(1S,7E,8aS)-7-[2-(2-Cyclohexylethinyl)benzyliden]-6-(butyl-amino)-1,2,3,4,6,7,8,8a-octahydro-8a-methylnaphthalen-1-ol,
(E,4aS)-6-{2-[2-(3-Fluorphenyl)ethinyl]benzyliden}-5-(butylamino)-decahydro-1,4a-dimethylnaphthalen-2-ol,
(1S,7E,8aS)-7-[2-(2-Cyclohexylethinyl)benzyliden]-6-(butylamino)-1,2,3,4,6,7,8,8a-octahydro-5,8a-dimethylnaphthalen-1-ol,
(E,1S,8aS)-7-[2-(3-Methoxyprop-1-inyl)benzyliden]-6-(butylamino)-decahydro-5,8a-dimethylnaphthalen-1-ol,
(E,4aS)-6-{2-[3-(Dimethylamino)prop-1-inyl]benzyliden}-5-(butylamino)-decahydro-1,4a-dimethylnaphthalen-2-ol,
(1S,7E,8aS)-7-[2-(Hex-1-inyl)-benzyliden]-6-(cyclohexyl-amino)-1,2,3,4,6,7,8,8a-octahydro-5,8a-dimethylnaphthalen-1-ol,
(1S,7E,8aS)-7-{2-[3-(Dimethylamino)prop-1-inyl]benzyliden}-6-(cyclohexylamino)-1,2,3,4,6,7,8,8a-octahydro-5,8a-dimethylnaphthalen-1-ol,
(E,4aS)-6-[2-(Oct-1-inyl)benzyliden]-5-(cyclohexylamino)-decahydro-1,4a-dimethylnaphthalen-2-ol,
(1S,7E,8aS)-7-{2-[3-(Dimethylamino)prop-1-inyl]benzyliden}-8a-benzyl-6-(cyclohexylamino)-1,2,3,4,6,7,8,8a-octahydro-5-methylnaphthalen-1-ol,
(3E,4aS,5S)-3-{2-[2-(3-Fluorphenyl)ethinyl]benzyliden}-4,4a,5,6,7,8-hexahydro-5-hydroxy-4a-methyl naphthalen-2(3H)-one,
(3E,4R,4aS,5S)-3-[2-(Hexadec-1-inyl)benzyliden]-4a-allyl-4,4a,5,6,7,8-hexahydro-5-hydroxy-4-methylnaphthalen-2(3H)-one,
(E,4aS,5S)-3-[2-(3-Methoxyprop-1-inyl)benzyliden]-octahydro-5-hydroxy-1,4a-dimethylnaphthalen-2(1H)-one,
(E,8aS)-2-{2-[3-(Dimethylamino)prop-1-inyl]benzyliden}-octahydro-6-hydroxy-5,8a-dimethylnaphthalen-1(2H)-one,
(3E,4aS,5S)-3-{2-[2-(3-Fluorphenyl)ethinyl]benzyliden}-4,4a,5,6,7,8-hexahydro-5-hydroxy-1,4a-dimethyl naphthalen-2(3H)-one,
(3E,4R,4aS,5S)-3-[2-(3-Methoxyprop-1-inyl)benzyliden]-4,4a,5,6,7,8-hexahydro-5-hydroxy-4,4a-dimethylnaphthalen-2(3H)-one,
(E,4aS,5S)-3-{2-[2-(3-Fluorphenyl)ethinyl]benzyliden}-octahydro-5-hydroxy-1,4a-dimethylnaphthalen-2(1H)-one,
(3E,4aS,5S)-3-[2-(Hexadec-1-inyl)benzyliden]-4,4a,5,6,7,8-hexahydro-5-hydroxy-4a-methylnaphthalen-2(3H)-one,
(3E,4R,4aS,5S)-3-{2-[2-(3-Fluorphenyl)ethinyl]benzyliden}-4a-allyl-4,4a,5,6,7,8-hexahydro-5-hydroxy-4-methylnaphthalen-2(3H)-one,
(3E,4aS,5S)-3-[2-(3-Methoxyprop-1-inyl)benzyliden]-4,4a,5,6,7,8-hexahydro-5-hydroxy-4a-methyl naphthalen-2(3H)-one,
(3E,4R,4aS,5S)-3-{2-[2-(3-Fluorphenyl)ethinyl]benzyliden}-4,4a,5,6,7,8-hexahydro-5-hydroxy-4,4a-dimethylnaphthalen-2(3H)-one,
(E,8aS)-2-[2-(Hex-1-inyl)benzyliden]-octahydro-6-hydroxy-5,8a-dimethylnaphthalen-1(2H)-one,
(3E,4aS,5S)-3-[2-(3-Methoxyprop-1-inyl)benzyliden]-4,4a,5,6,7,8-hexahydro-5-hydroxy-1,4a-dimethylnaphthalen-2(3H)-one,
(3E,4aS,5S)-3-[2-(3-Methoxyprop-1-inyl)benzyliden]-4a-benzyl-4,4a,5,6,7,8-hexahydro-5-hydroxy-1-methylnaphthalen-2(3H)-one,
(1S,6E,7E,8S,8aS)-7-[3-(2-Cyclohexylethinyl)benzyliden]-8a-allyl-6-(butylimino)-1,2,3,4,6,7,8,8a-octahydro-8-methylnaphthalen-1-ol,
(E,4aS)-6-[3-(3-Methoxyprop-1-inyl)benzyliden]-5-(butylamino)-decahydro-1,4a-dimethylnaphthalen-2-ol,
(E,4aS)-6-[3-(3-Hydroxy-3-methylbut-1-inyl)benzyliden]-5-(butylamino)-decahydro-1,4a-dimethylnaphthalen-2-ol,
(1S,7E,8aS)-7-[3-(2-Cyclohexylethinyl)benzyliden]-6-(butyl-amino)-1,2,3,4,6,7,8,8a-octahydro-8a-methylnaphthalen-1-ol,
(E,4aS)-6-{3-[2-(2-Fluorphenyl)ethinyl]benzyliden}-5-(butylamino)-decahydro-1,4a-dimethylnaphthalen-2-ol,
(E,4aS)-6-[3-(Hex-1-inyl)benzyliden]-5-(butylamino)-decahydro-1,4a-dimethylnaphthalen-2-ol,
(E,1S,8aS)-7-[3-(3-Methoxyprop-1-inyl)benzyliden]-6-(butylamino)-decahydro-5,8a-dimethylnaphthalen-1-ol,
(E,1S,8aS)-7-[3-(3-Methoxyprop-1-inyl)benzyliden]-6-(cyclohexylamino)-decahydro-5,8a-dimethylnaphthalen-1-ol,
(E,1S,8S,8aR)-7-{3-[2-(3-Fluorphenyl)ethinyl]benzyliden}-8a-allyl-6-(cyclohexylamino)-decahydro-5,8-dimethylnaphthalen-1-ol,
(3E,4aS,5S)-3-{3-[2-(3-Fluorphenyl)ethinyl]benzyliden}-4,4a,5,6,7,8-hexahydro-5-hydroxy-4a-methylnaphthalen-2(3H)-one,
(E,4R,4aR,5S)-3-[3-(3-Methoxyprop-1-inyl)benzyliden]-4a-allyl-octahydro-5-hydroxy-1,4-dimethylnaphthalen-2(1H)-one,
(3E,4aS,5S)-3-{3-[2-(3-Fluorphenyl)ethinyl]benzyliden}-4,4a,5,6,7,8-hexahydro-5-hydroxy-1,4a-dimethylnaphthalen-2(3H)-one,
(E,8aS)-2-[3-(3-Methoxyprop-1-inyl)benzyliden]-octahydro-6-hydroxy-5,8a-dimethylnaphthalen-1(2H)-one,
(E,4aS,5S)-3-{3-[2-(3-Fluorphenyl)ethinyl]benzyliden}-octahydro-5-hydroxy-1,4a-dimethylnaphthalen-2(1H)-one,
(E,4R,4aR,5S)-3-{3-[2-(3-Fluorphenyl)ethinyl]benzyliden}-4a-allyl-octahydro-5-hydroxy-1,4-dimethylnaphthalen-2(1H)-one,
(3E,4aS,5S)-3-[3-(3-Methoxyprop-1-inyl)benzyliden]-4,4a,5,6,7,8-hexahydro-5-hydroxy-4a-methylnaphthalen-2(3H)-one,
(3E,4aS,5S)-3-[3-(Hexadec-1-inyl)benzyliden]-4,4a,5,6,7,8-hexahydro-5-hydroxy-1,4a-dimethylnaphthalen-2(3H)-one,
(3E,4R,4aS,5S)-3-{3-[2-(3-Fluorphenyl)ethinyl]benzyliden}-4,4a,5,6,7,8-hexahydro-5-hydroxy-4,4a-dimethylnaphthalen-2(3H)-one,
(3E,4aS,5S)-3-[3-(3-Methoxyprop-1-inyl)benzyliden]-4,4a,5,6,7,8-hexahydro-5-hydroxy-1,4a-dimethylnaphthalen-2(3H)-one,
(3E,4aS,5S)-3-[3-(3-Methoxyprop-1-inyl)benzyliden]-4a-benzyl-4,4a,5,6,7,8-hexahydro-5-hydroxy-1-methylnaphthalen-2(3H)-one,
(E,1S,8aS)-7-[3-(3-Methoxyprop-1-inyl)benzyliden]-6-(butylamino)-decahydro-5,8a-dimethylnaphthalen-1-ol,
(E,1S,8aS)-7-[3-(2-Cyclohexylethinyl)benzyliden]-6-(butylamino)-decahydro-5,8a-dimethylnaphthalen-1-ol,
(E,4aS)-6-[3-(3-Methoxyprop-1-inyl)benzyliden]-5-(butylamino)-decahydro-1,4a-dimethylnaphthalen-2-ol,
(E,1S,8S,8aR)-7-{3-[2-(3-Fluorphenyl)ethinyl]benzyliden}-8a-allyl-6-(butylamino)-decahydro-5,8-dimethylnaphthalen-1-ol,
(E,4aS)-6-[3-(3-Hydroxy-3-methylbut-1-inyl)benzyliden]-5-(butylamino)-decahydro-1,4a-dimethylnaphthalen-2-ol,
(1S,7E,8aS)-7-[3-(2-Cyclohexylethinyl)benzyliden]-6-(butyl-amino)-1,2,3,4,6,7,8,8a-octahydro-8a-methylnaphthalen-1-ol,
(E,4aS)-6-{3-[2-(3-Fluorphenyl)ethinyl]benzyliden}-5-(butylamino)-decahydro-1,4a-dimethylnaphthalen-2-ol,
(1S,7E,8aS)-7-[3-(3-Hydroxy-3-methylbut-1-inyl)benzyliden]-6-(butylamino)-1,2,3,4,6,7,8,8a-octahydro-8a-methylnaphthalen-1-ol,
(E, 1S,8S,8aR)-7-[3-(3-Methoxyprop-1-inyl)benzyliden]-8a-allyl-6-(butylamino)-decahydro-5,8-dimethylnaphthalen-1-ol,
(E,4aS)-6-{3-[3-(Dimethylamino)prop-1-inyl]benzyliden}-5-(butylamino)-decahydro-1,4a-dimethylnaphthalen-2-ol,
(E,4aS)-6-{3-[2-(3-Fluorphenyl)ethinyl]benzyliden}-5-(cyclohexylamino)-decahydro-1,4a-dimethylnaphthalen-2-ol,
(1S,7E,8aS)-7-{3-[2-(3-Fluorphenyl)ethinyl]benzyliden}-8a-benzyl-6-(cyclohexylamino)-1,2,3,4,6,7,8,8a-octahydro-5-methylnaphthalen-1-ol,
(E,4aS)-6-[3-(3-Methoxyprop-1-inyl)benzyliden]-5-(cyclohexylamino)-decahydro-1,4a-dimethylnaphthalen-2-ol,
(E,4aS)-6-[3-(2-Cyclohexylethinyl)benzyliden]-5-(cyclohexylamino)-decahydro-1,4a-dimethylnaphthalen-2-ol,
(E,4aS)-6-[3-(3-Methyl but-3-en-1-inyl)benzyliden]-5-(cyclohexylamino)-decahydro-1,4a-dimethylnaphthalen-2-ol,
(3E,4aS,5S)-3-{4-[2-(3-Fluorphenyl)ethinyl]benzyliden}-4,4a,5,6,7,8-hexahydro-5-hydroxy-4a-methyl naphthalen-2(3H)-one,
(E,4aS,5S)-3-[4-(3-Methoxyprop-1-inyl)benzyliden]-octahydro-5-hydroxy-1,4a-dimethylnaphthalen-2(1H)-one,
(3E,4R,4aS,5S)-3-{4-[3-(Dimethylamino)prop-1-inyl]benzyliden}-4,4a,5,6,7,8-hexahydro-5-hydroxy-4,4a-dimethylnaphthalen-2(3H)-one,
(3E,4R,4aS,5S)-3-[4-(3-Methoxyprop-1-inyl)benzyliden]-4a-allyl-4,4a,5,6,7,8-hexahydro-5-hydroxy-4-methylnaphthalen-2(3H)-one,
(3E,4aS,5S)-3-{4-[2-(3-Fluorphenyl)ethinyl]benzyliden}-4,4a,5,6,7,8-hexahydro-5-hydroxy-1,4a-dimethylnaphthalen-2(3H)-one,
(3E,4aS,5S)-3-{4-[2-(3-Fluorphenyl)ethinyl]benzyliden}-4a-benzyl-4,4a,5,6,7,8-hexahydro-5-hydroxy-1-methylnaphthalen-2(3H)-one,
(3E,4R,4aS,5S)-3-[4-(2-Cyclohexylethinyl)benzyliden]-4a-allyl-4,4a,5,6,7,8-hexahydro-5-hydroxy-4-methylnaphthalen-2(3H)-one,
(E,4aS,5S)-3-{4-[2-(3-Fluorphenyl)ethinyl]benzyliden}-octahydro-5-hydroxy-1,4a-dimethylnaphthalen-2(1H)-one,
(3E,4R,4aS,5S)-3-[4-(3-Cyclohexylprop-1-inyl)benzyliden]-4,4a,5,6,7,8-hexahydro-5-hydroxy-4,4a-dimethylnaphthalen-2(3H)-one,
(3E,4aS,5S)-3-[4-(3-Methoxyprop-1-inyl)benzyliden]-4,4a,5,6,7,8-hexahydro-5-hydroxy-4a-methylnaphthalen-2(3H)-one,
(E,8aS)-2-{4-[2-(3-Fluorphenyl)-ethinyl]benzyliden}-octahydro-6-hydroxy-5,8a-dimethylnaphthalen-1(2H)-one,
(3E,4aS,5S)-3-[4-(3-Methoxyprop-1-inyl)benzyliden]-4,4a,5,6,7,8-hexahydro-5-hydroxy-1,4a-dimethylnaphthalen-2(3H)-one,
(3E,4aS,5S)-3-[4-(3-Methoxyprop-1-inyl)benzyliden]-4a-benzyl-4,4a,5,6,7,8-hexahydro-5-hydroxy-1-methylnaphthalen-2(3H)-one,
(3E,4R,4aS,5S)-3-{4-[3-(Dimethylamino) prop-1-inyl]benzyliden}-4a-allyl-4,4a,5,6,7,8-hexahydro-5-hydroxy-4-methylnaphthalen-2(3H)-one,
(1S,8aS)-6-(Diethylamino)-1,2,3,4,6,7,8,8a-octahydro-5,8a-dimethylnaphthalen-1-ol,
(1S,8S,8aS)-6-(Diethylamino)-1,2,3,4,6,7,8,8a-octahydro-8,8a-dimethylnaphthalen-1-ol,
(1S,8aS)-6-(Diethylamino)-1,2,3,4,6,7,8,8a-octahydro-8a-methylnaphthalen-1-ol,
(1S,8aR)-8a-Benzyl-6-(diethylamino)-1,2,3,4,6,7,8,8a-octahydro-5-methylnaphthalen-1-ol,
(1S,8S,8aS)-8a-Allyl-6-(diethylamino)-1,2,3,4,6,7,8,8a-octahydro-8-methylnaphthalen-1-ol,
(6E,4aS)-2-Furan-2-ylmethylene-5,8a-dimethyl-6-methylene-3,4,6,7,8,8a-hexahydro-2H-naphthalen-1-one,
2-Furan-2-ylmethylene-8-methyl-3,4,4a,5,8,8a-hexahydro-2H-naphthalen-1-one,
(4aS,5S)-5-Hydroxy-1,4a-dimethyl-3-(4-pyridin-4-ylethynyl-benzylidene)-4,4a,5,6,7,8-hexahydro-3H-naphthalen-2-one,
(4aS,5S)-3-[4-(3-Dimethylamino-prop-1-ynyl)-benzylidene]-5-hydroxy-1,4a-dimethyl-4,4a,5,6,7,8-hexahydro-3H-naphthalen-2-one,
(4aS,5S)-5-Hydroxy-1,4a-dimethyl-1-(3-phenyl-propyl)-octahydro-naphthalen-2-one,
(4aS,5S)-5-Hydroxy-1-(3-iodo-benzyl)-1,4a-dimethyl-octahydro-naphthalen-2-one,
(4aS,5S)-3-(4-Cyclohexylamino-benzylidene)-5-hydroxy-1,4a-dimethyl-4,4a,5,6,7,8-hexahydro-3H-naphthalen-2-one,
(4aS)-5,8a-Dimethyl-7-(5-methyl-furan-2-ylmethylene)-hexahydro-naphthalen-1,6-dione,
(2E,4aS)-5-(1,4a-Dimethyl-5-oxo-4,4a,5,6,7,8-hexahydro-3H-naphthalen-2-ylidene)-5H-furan-2-one,
(7′E)-7′-(4-iodo-benzylidene)-5′,8a′-dimethyl hexahydro-5′-(2-phenylethyl)-2′H-spiro[1,3-dioxolane-2,1′-naphthalen]-6′(5′H)-one,
2-Furan-2-ylmethylene-3,4,4a,5,8,8a-hexahydro-2H-naphthalen-1-one,
(4aS)-7-Benzylidene-5,8a-dimethyl-hexahydro-naphthalen-1,6-dione,
(4aS,5S)-5-Hydroxy-1,4a-dimethyl-1-(2-nitro-benzyl)-octahydro-naphthalen-2-one,
(4aS,5S)-5-Hydroxy-1-isobutyl-1,4a-dimethyl-1-octahydro-naphthalen-2-one,
8-Methyl-3,4,4a,5,8,8a-hexahydro-2H-naphthalen-1-one,
(3E,4aS)-5,8a-Dimethyl-7-(2,4,6-trimethoxy-benzylidene)-3,4,8,8a-tetrahydro-2H,7H-naphthalen-1,6-dione,
(3E,3′E,4aS,4a′S)-1,3-bis[5,8a-dimethyl-3,4,8,8a-tetrahydro-2H,7H-naphthalen-1,6-dione]-benzene,
(4aS)-5-Butyliminomethyl-8a-methyl-3,4,8,8a-tetrahydro-2H,7H-naphthalen-1,6-dione,
(2E,4aS,5S)-6-Ethylidene-5,8a-dimethyl-1,2,3,4,6,7,8,8a-octahydro-naphthalen-1-ol,
(4aS,5S)-5-Hydroxy-3-[1-(4-iodo-phenyl)-propyl]-1,4a-dimethyl-4,4a,5,6,7,8-hexahydro-3H-naphthalen-2-one,
(4aS,5S)-5-Hydroxy-1,4a-dimethyl-4,4a,5,6,7,8-hexahydro-3H-naphthalen-2-one,
(4aS,5S)-1-Butyl-5-hydroxy-1,4a-dimethyl-1-octahydro-naphthalen-2-one,
(5E)-5-(5′, 8a′-dimethyl-3′,4′, 8′,8a′-tetrahydro-2′H-spiro[1,3-dioxolane-2,1′-naphthalen]-6′(7′H)-ylidene)furan-2(5H)-one,
(7′E)-5′-(3,5-Dibromobenzyl)-7′-(4-iodo-benzylidene)-5′, 8a′-dimethylhexahydro-2′H-spiro[1,3-dioxolane-2,1′-naphthalen]-6′(5′H)-one,
5-Hydroxy-7-isopropyl-1-methyl-4,4a,5,6,7,8,-hexahydro-3H-naphthalen-2-one,
(4aS,5S)-5-Hydroxy-1,4a-dimethyl-1-octahydro-naphthalen-2-one.

4. Compound according to any previous claim for use as a pharmaceutically active agent.

5. Use of a compound according to any one of claims 1-4 as an inhibitor for at least one enzyme selected from the group comprising Cdc25A, AChE, 11βHSD1, 11βHSD2, MtPTPA, PP1, VHR, PTP1B.

6. Use of a compound according to any one of claims 1-4 for prophylaxis and treatment of diseases associated with the enzymes Cdc25A, AChE, 11βHSD1, 11βHSD2, MtPTPA, PP1, VHR, PTP1B or curable by inhibition of at least one of the enzymes Cdc25A, AChE, 11βHSD1, or 11βHSD2, MtPTPA, PP1, VHR, PTP1B.

7. Use of a compound according to any one of claims 1-4 for prophylaxis and treatment of diabetes mellitus type I, diabetes mellitus type II, tuberculosis, proliferative diseases, cancer, infectious diseases, neurodegenerative diseases, obesity, cognitive dysfunctions, wound healing, cardiovascular diseases and disorders, glucocorticoid dysfunction and metabolic syndromes or as angiogenesis inhibitor.

8. Use according to claim 7, where the neurodegenerative disease is selected from the group comprising: Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA).

9. Use according to claim 7, wherein the proliferative disease or cancer is selected from the group comprising:
adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

10. Use according to claim 7, wherein the infectious disease including opportunistic infection is selected from the group comprising:
AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever, tuberculosis, leprosy, mycobacteria-induced meningitis.

11. Use according to claim 7, where the cognitive disorder is selected from the group comprising anxiety disorders, delirium, dementia, amnestic disorders, dissociative disorders, eating disorders, mood disorders, schizophrenia, psychotic disorders, sexual and gender identity disorders, sleep disorders, somatoform disorders, acute stress disorder, obsessive-compulsive disorder, panic disorder, posttraumatic stress disorder, specific phobia, social phobia, substance withdrawal delirium, Alzheimer's disease, Creutzfeldt-Jakob disease, head trauma, Huntington's disease, HIV disease, Parkinson's disease, Pick's disease, learning disorders, motor skills disorders, developmental coordination disorder, communication disorders, phonological disorder, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, pica, rumination disorder, tic disorders, chronic motor or vocal tic disorder, Tourette's disorder, elimination disorders, encopresis, enuresis, selective mutism, separation anxiety disorder, dissociative amnesia, depersonalization disorder, dissociative fugue, dissociative identity disorder, anorexia nervosa, bulimia nervosa, bipolar disorders, schizophreniform disorder, schizoaffective disorder, delusional disorder, psychotic disorder, shared psychotic disorder, delusions, hallucinations, substance-induced psychotic disorder, orgasmic disorders, sexual pain disorders, dyspareunia, vaginismus, sexual dysfunction, paraphilias, dyssomnias, breathing-related sleep disorder, circadian rhythm sleep disorder, hypersomnia, insomnia, narcolepsy, dyssomnia, parasomnias, nightmare disorder, sleep terror disorder, sleepwalking disorder, parasomnia, body dysmorphic disorder, conversion disorder, hypochondriasis, pain disorder, somatization disorder, alcohol related disorders, amphetamine related disorders, caffeine related disorders, cannabis related disorders, cocaine related disorders, hallucinogen related disorders, inhalant related disorders, nicotine related disorders, opioid related disorders, phencyclidine-related disorder, abuse, persisting amnestic disorder, intoxication, withdrawal.

12. Use according to claim 7, where the cardiovascular disease, cardiovascular disorder, glucocorticoid dysfunction or metabolic syndrome is selected from the group comprising metabolic syndrome, visceral obesity, insulin resistance, dyslipidemia, obesity, impaired glucose tolerance, hypertriglyceridemia, Cushing's syndrome, Addison's disease, glucocorticoid hypersensitivity, glucocorticoid resistance, hypercortisolism, the dysmetabolic syndrome, diabetes type 2, melancholic depression, osteoporosis, anxiety, insomnia, asthenia, depression, memory dysfunction, executive dysfunction, fatigue, hyperalgesia, poor quality sleep, insulin resistance, atrophy (sarcopenia), growth stunting, adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, atherosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

13. Use of a compound according to any one of claims 1-4 in combination with an anti-HIV drug, antiproliferative drug, cytotoxic or cytostatic drug, ganciclovir, foscarnet, cidofovir, valganciclovir, fomivirsen, penciclovir or valaciclovir.

14. Pharmaceutical composition comprising at least one compound according to any one of claims 1 to 4 as an active ingredient, together with at least one pharmaceutically acceptable carrier, excipient and/or diluents.
